(19) United States
(12) Patent Application Publication
(10) Pub. No.: US 2021/0355106 A1
(43) Pub. Date: Nov. 18, 2021

(54) NEW AMINO-PYRIMIDONYL-PIPERIDINYL DERIVATIVES, A PROCESS FOR THEIR PREPARATION AND PHARMACEUTICAL COMPOSITIONS CONTAINING THEM

(71) Applicants: LES LABORATOIRES SERVIER, SURESNES CEDEX (FR); VERNALIS (R&D) LTD, Cambridge (GB)

(72) Inventors: Csaba WÉBER, Pilisszentlászló (HU); András KOTSCHY, Törökbálint (HU); Attila VASAS, Fót (HU); Árpád KISS, Budakalász (HU); Balázs MOLNÁR, Isaszeg (HU); Andrea FIUMANA, Ely (GB); Alba MACIAS, Cambridge (GB); James Brooke MURRAY, Linton (GB); Didier DEMARLES, Checy (FR); Lisa IVANSCHITZ, Massy (FR); Olivier GENESTE, Rueil-Maimaison (FR); Kristóf HEGEDÜS, Budapest (HU); Péter SPRÁNITZ, Budapest (HU); Tibor SOÓS, Budapest (HU)

(21) Appl. No.: 17/282,898

(22) PCT Filed: Oct. 18, 2019
(86) PCT No.: PCT/EP2019/078318

§ 371 Date: Apr. 5, 2021

(30) Foreign Application Priority Data
Oct. 19, 2018 (EP) 18306377.5
Jul. 12, 2019 (EP) 19305936.7

(51) Int. Cl. C07D 401/14; A61P 35/00; A61K 45/06; A61K 31/513; C07D 417/14; A61K 31/517; C07D 409/14; C07D 405/14

(52) U.S. Cl. CPC C07D 401/14; A61P 35/00; A61K 45/06; A61K 31/513; C07B 2200/07; A61K 31/517; C07D 409/14; C07D 405/14; C07D 417/14

(57) ABSTRACT

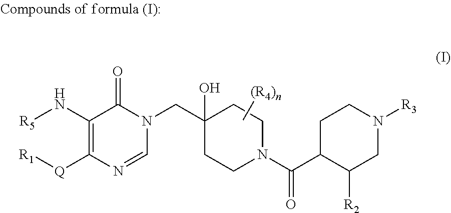

wherein R₁, R₂, R₃, R₄, R₅, n and Q are as defined in the description.

The present invention relates to new amino-pyrimidonyl-piperidinyl derivatives, to a process for their preparation and to pharmaceutical compositions containing them.

The compounds of the present invention are new and have very valuable pharmacological characteristics in the field of apoptosis and oncology.

Ubiquitination is a process controlling essential cellular functions such as protein turnover and homeostasis, protein activation and localisation. Ubiquitin is a 76 amino acids polypeptide which is covalently attached to postranslationnaly modified protein substrates via an isopeptide bond. Deubiquinating enzymes (DUBs) are in majority cysteine proteases that cleave the ubiquitin-ubiquitin bond or ubiquitin-protein bond at the Cter glycine of Ubiquitin. Approximately 100 DUBs regulate the thousands ubiquitinated proteins and then some redundancy of deubiquitinase substrates regulation are observed.

Dysregulation of DUBs have been associated with several diseases such as neurodegenerative and infectious diseases (Edelman et al. Expert Rev. Mol. Med. 2011, 13, 1-17) and human malignancies (Pal et al., Cancer Res. 2014, 74, 4955-4966). Accordingly, overexpression of DUBs or increase of their activity have been associated to numerous types of cancers (Luise et al., Plos One 2011, 6, e15891: Rolen et al., Mol. Carcinog. 2006, 45, 260-269) and poor prognosis.

Ubiquitin Specific Protease 7 (USP7), also known as Herpes-virus-Associated Ubiquitin-Specific Protease (HAUSP), belongs to the deubiquitinating family. USP7 has been reported to stabilize numerous oncogenes involved in survival and proliferations via cell cycle progression, apoptosis, DNA repair, DNA replication and epigenetic factors regulation (Nicholson et al., Cell Biochem. Biophys. 2011, 60, 61-68). In addition, USP7 has been shown to regulate immune response via inflammation and Treg modulation (Van Loosdregt et al., Immunity 2013, 39, 259-27: Colleran et al., Proc. Natl. Acad. Sci. USA 2013, 110, 618-623). USP7 has also been implicated in other pathologic states such as neurodevelopmental disorder (Hao et al., Mol. Cell 2015, 59, 956-969) and viral infection (Holowaty et al., Biochem. Soc. Trans. 2004, 32, 731-732).

USP7 overexpression has been associated with late stages of cancers and poor prognosis in lung, neuroblastoma, myeloma, prostate, colon and breast cancers. Numerous USP7 inhibitors have been recently published in the literature (Turnbull et al. Nature 2017, 550, 481-486; Kateeaya et al, Nature 2017, 550, 534-538: Gavory et al., Nat Chem. Biol. 2018, 14, 118-125: O'Dowd et al., ACS Med. Chem. Lett. 2018, 9, 238-243: Pozhidaeva et al. Cell Chem. Biol. 2017, 24, 1501-1512: Lamberto et al., Cell Chem. Biol. 2017, 24, 1490-1500; PCT/EP2017/064062; PCT/EP2017/064067) and, particularly, pyrimidonyl derivatives claimed as USP7 inhibitors have been disclosed in PCT/GB2017/053175. However. PCT/GB2017/053175 shows that 5,6-disubstituted pyrimidonyl derivatives provide compounds with weakest affinity on USP7. Despite an intense research in the field, no USP7 inhibitors have entered the clinic (Kemp et al, Progress in Medicinal Chemistry 2016, 55, 149-192: Wu et al. J. Med. Chem. 2018, 61, 422-443). There is, therefore, a therapeutic need for compounds that inhibit the activity of the protein USP7.

In addition to being new and very potent on their target, the compounds of the present invention have pro-apoptotic and/or anti-proliferative properties making it possible to use them in pathologies involving a defect in apoptosis, such as, for example, in the treatment of cancer and of immune and auto-immune diseases.

The present invention relates more especially to compounds of formula (I):

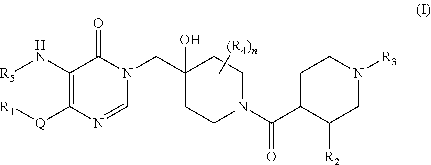

wherein:
Q represents an oxygen atom or a sulphur atom.
R₁ represents a cycloalkyl group, a heterocycloalkyl group, an aryl group, or a heteroaryl group,
R₂ represents an aryl group or a heteroaryl group,
R₃ represents a hydrogen atom, a linear or branched (C₁-C₆)alkyl group, a linear or branched halo(C₁-C₆)alkyl, a linear or branched hydroxy(C₁-C₆)alkyl group, a —C(O)—R₈ group, a —C(O)—OR₈ group, a —C(O)—NH—R₈ group, or a

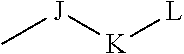

group,
R₄ represents a hydrogen atom or a halogen atom,
R₅ represents a hydrogen atom, a linear or branched (C₁-C₆)alkyl group, a linear or branched halo(C₁-C₆)alkyl group, or an aryl(C₁-C₆)alkyl group,
n is in integer equal to 0, 1 or 2.
J represents a —C(O)— group, a —CH(R₆)— group a —SO₂— group, a —C(X)—N(R₇)— group, a —CH₂—C(O)—N(R₇) group, or a

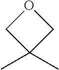

group.
R₆ represents a hydrogen atom, a linear or branched (C₁-C₆)alkyl group, or a —C(O)—OR₈ group,
R₇ represent a hydrogen atom or a linear or branched (C₁-C₆)alkyl group,
R₈ represent a hydrogen atom, a linear or branched (C₁-C₆)alkyl group, or a linear or branched halo(C₁-C₆)alkyl group,
K represents a bond or a -Cy₁- group,
L represents a linear or branched (C₁-C₆)alkyl group, a -Cy₂ group, or a —C(R₉)₂-Cy₂ group,
X represents an oxygen atom or a sulphur atom,
R₉ represents a hydrogen atom or a halogen atom,
Cy₁ represents a cycloalkyl group, a heterocycloalkyl group, an aryl group, or a heteroaryl group, which is linked to the group J and to the group L,
Cy₂ represents a cycloalkyl group, a heterocycloalkyl group, an aryl group, or a heteroaryl group.
   it being understood that
“aryl” means a phenyl, naphthyl, or indanyl group.
“heteroaryl” means any mono- or fused bi-cyclic group composed of from 5 to 10 ring members, having at least one aromatic moiety and containing from 1 to 3 heteroatoms selected from oxygen, sulphur and nitrogen,
“cycloalkyl” means any mono- or fused bi-cyclic non-aromatic carbocyclic group containing from 3 to 7 ring members.
“heterocycloalkyl” means any non-aromatic mono- or fused bi-cyclic group containing from 3 to 10 ring members, and containing from 1 to 3 heteroatoms selected from oxygen, sulphur and nitrogen,
   it being possible for the aryl, heteroaryl, cycloalkyl and heterocycloalkyl groups so defined to be substituted by from 1 to 4 groups selected from linear or branched (C₁-C₆)alkyl, linear or branched (C₂-C₆)alkenyl, linear or branched (C₂-C₆)alkynyl, linear or branched halo(C₁-C₆)alkyl. —Y₁—OR′, —Y₁—NR′R″, —Y₁—S(O)ₘ—R′, oxo, N-oxide (where appropriate), pentafluorosulfide, nitro, —Y₁—CN, —C(O)—R′, —C(O)—OR′, —O—C(O)—R′, —Y₁—C(O)—NR′R″, —Y₁—NR′—C(O)—R″, —Y₁—NR′—C(O)—OR″, halogen, cyclopropyl and pyridinyl which can be substituted by a linear or branched (C₁-C₆)alkyl group,
   it being understood that:
Y₁ represents a bond, a linear or branched (C₁-C₄)alkylene group, or a linear or branched halo(C₁-C₆)alkylene group,
R′ and R″ independently of one another, represent a hydrogen atom, a linear or branched (C₁-C₆)alkyl group, a linear or branched (C₂-C₆)alkenyl group, a linear or branched (C₂-C₆)alkynyl group, a linear or branched (C₁-C₆)alkoxy group, a linear or branched halo(C₁-C₆)alkyl, a linear or branched hydroxy(C₁-C₆)alkyl group, a linear or branched (C₁-C₆)alkoxy(C₁-C₆)alkyl group, a formyl group, a phenyl group, a benzyl group, a cyclopropyl group, a cyclopropylmethyl group, a tetrahydropyranyl group, or the pair (R′, R″) together with the nitrogen atom to which they are attached forms a non-aromatic ring composed of from 4 to 7 ring members, which may contain in addition to the nitrogen a second heteroatom selected from oxygen and nitrogen, it being understood that the second nitrogen in question may be substituted by from 1 to 2 groups representing a hydrogen atom or a linear or branched (C₁-C₆)alkyl group.
m is an integer equal to 0, 1 and 2,
   their enantionmers, diastereoisomers and addition salts thereof with a pharmaceutically acceptable acid or base,

In a preferred embodiment of the invention, the present invention relates to compounds of formula (I) wherein:
Q represents an oxygen atom,
R₁ represents an aryl group or a heteroaryl group,
R₂ represents an aryl group,
R₃ represents a hydrogen atom, a linear or branched hydroxy(C₁-C₆)alkyl group, a —C(O)—OR₈ group, a —C(O)—NH—R₈ group, or a

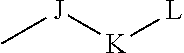

group,
R₅ represents a hydrogen atom,
R₆ represents a hydrogen atom or a —C(O)—OR₈ group.
R₈ represents a linear or branched (C₁-C₆)alkyl group or a linear or branched halo(C₁-C₆)alky group,
Cy₁ represents a cycloalkyl group, an aryl group, or a heteroaryl group, which is linked to the group J and to the group L,
   it being possible for the aryl, heteroaryl, cycloalkyl and heterocycloalkyl groups so defined to be substituted by from 1 to 4 groups selected from linear or branched (C₁-C₆)alkyl, linear or branched halo(C₁-C₆)alkyl, —Y₁—OR′, —Y₁—NR′R″, oxo, —Y₁—CN, —Y₁—NR′—C(O)—OR″, halogen, or cyclopropyl,
   it being, understood that R′ and R″ independently of one another, represent a hydrogen atom, a linear or branched (C₁-C₆)alkyl group, a linear or branched halo(C₁-C₆)alkyl, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by from 1 to 2 groups representing a hydrogen atom, or a linear or branched (C₁-C₆)alkyl group.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

Among the heteroaryl groups there may be mentioned, without implying any limitation, pyrrolyl, furyl, thienyl, thiazolyl, isothiazolyl, oxazolyl, isoxazolyl, pyrazolyl, imidazolyl, pyridinyl (also known as pyridyl), pyrazinyl, pyridazinyl, pyrimidinyl, pyridinonyl, indolyl, dihydroindolyl, dihydroisoindolyl, indazolyl, dihydrocyclopentathienyl, benzothienyl, tetrahydrobenzothienyl, benzofuranyl, imidazopyridinyl, benzotriazolyl, benzodioxolyl, dihydrobentodioxinyl, quinolinyl, isoquinolinyl, tetrahydroquinolinyl, tetrahydroisoquinolinyl, quinoxalinyl, dihydroquinoxalinyl, dihydrothienodioxinyl, quinazolinonyl, pyrrolopyridazinyl, pyrrolopyridinyl, dihydropyrrolizinyl, tetahydroindolizinyl, etc.

Among the cycloalkyl groups there may be mentioned, without implying any limitation, cyclopropyl, cyclobutyl, cyclopentyl, cyclohexyl, etc.

Among the heterocycloalkyl groups there may be mentioned, without implying any limitation, pyrrolidinyl, tetrahydropyranyl, piperidinyl, piperazinyl, morpholinyl, etc.

Advantageously, the compounds of formula (I) display a trans configuration as follows:

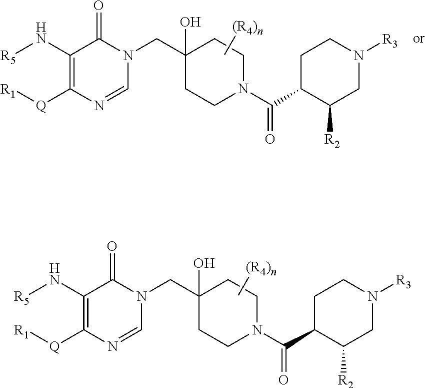

wherein R₁, R₂, R₃, R₄, R₅, Q and n are as defined for formula (I).

More preferably, the compounds of formula (I) display a trans configuration as follows:

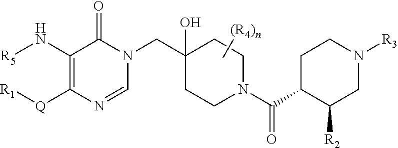

wherein R₁, R₂, R₃, R₄, R₅, Q and n are as defined for formula (I).

In another embodiment, when R₄ represents a halogen atom and n is an integer equal to 1 or 2, a new asymmetric carbon is created providing two possible isomers as follows:

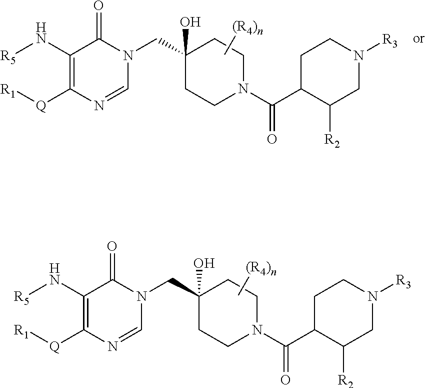

wherein R₁, R₂, R₃, R₅, Q and n are as defined for formula (I) and R₄ is as defined hereinbefore.

Preferably, when R₄ represents a halogen atom and n is an integer equal to 2, having the following formula:

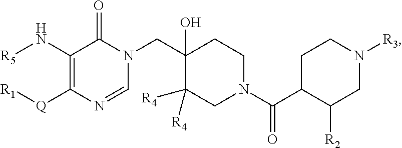

the preferred isomer has the S-configuration as follows:

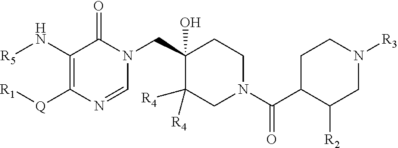

wherein R₁, R₂, R₃, R₅ and Q are as defined for formula (I) and R₄ is as defined hereinbefore.

Q advantageously represents an oxygen atom.

R₁ preferably represents an aryl group or a heteroaryl group. More preferably, R₁ represents a phenyl group or a pyridinyl group. Even more preferably, R₁ represents a phenyl group. In a preferred embodiment of the invention, R₄ represents a phenyl group which is substituted by from 1 to 2 groups selected from linear or branched halo(C₁-C₆)alkyl —Y—OR′, —Y—NR′R″, —Y₁—CN, —Y₁—NR′—C(O)—OR″, halogen, cyclopropyl, in which Y₁, R′ and R″ are as defined for formula (I). More advantageously, R₁ represents a phenyl group which is substituted by from 1 to 2 groups selected from fluorine, chlorine, methoxy, trifluoromethyl, trifluoromethoxy, aminomethyl or tert-butoxycarbonylaminomethyl.

R₂ preferably represents an aryl group. More preferably, R₂ represents a phenyl group.

R₃ preferably represents a hydrogen atom, a linear or branched hydroxy(C₁-C₆)alkyl a —C(O)—OR₈ group, a —C(O)—NH—R₈ group, or a

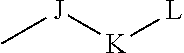

group.

More preferably, R₃ represents a

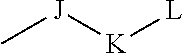

group.

Advantageously, R₄ represents a hydrogen atom or a fluorine atom. More preferably, R₄ represents a hydrogen atom. In another embodiment, when R₄ represents a fluorine atom and n is equal to 2, both fluorine atoms preferably represent a gem-difluoro group.

Preferably, R₅ represents a hydrogen atom.

Preferably, R₆ represents a hydrogen atom or a —C(O)—ORₓ group. More preferably, R₆ represents a hydrogen atom.

In the preferred compounds of the invention, J represents a —C(O)— group, a —CH₂— group, a —CH[C(O)—O—CH₂—CH₃]— group, a —SO₂— group, a —CH₂—C(O)—N(R₇)— group a —C(X)—N(R₇)— group, or a

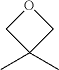

group.

Preferably, J represents a —C(O)— group, a —CH₂— group, a —SO₂— group, or a —C(O)—NH— group.

More preferably, J represents a —C(O)— group. Advantageously. J represents a —CH₂— group.

In another preferred embodiment, J represents a —C(O)—NH— group.

K preferably represents a bond or a -Cy₁- group selected from a phenyl group, a pyrrolyl group, a thienyl group, a thiazolyl group, an oxazolyl group, an imidazolyl group, a pyrazolyl group, a pyridinyl group, a pyrimidinyl group, a dihydrothienodioxinyl group, a cyclopropyl group, or a cyclobutyl group. More preferably, K preferably represents a bond or a -Cy₁-group selected from a phenyl group, a thienyl group, a thiazolyl group, an imidazolyl group, a pyridinyl group, a pyrimidinyl group, or a dihydrothienodioxinyl group.

Preferably, Cy₁ represents a cycloalkyl group, an aryl group, or a heteroaryl group. Preferably, Cy₁ represents a cycloalkyl group, an aryl group, or a heteroaryl group which are substituted by 1, 2 or 3 groups selected from linear or branched (C₁-C₆)alkyl, linear or branched halo(C₁-C₆)alkyl, —Y₁—OR′, —Y₁—NR′R″, oxo, halogen, in which R′ and R″ independently of one another represent a hydrogen atom, a linear or branched (C₁-C₆)alkyl group, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by a linear or branched (C₁-C₆)alkyl group. Preferably, Cy₁ represents a phenyl group, a pyrrolyl group, a thienyl group, a thiazolyl group, an oxazolyl group, an imidazolyl group, a pyrazolyl group, a pyridinyl group, a pyrimidinyl group, a dihydrothienodioxinyl group, a cyclopropyl group, or a cyclobutyl group, said groups are substituted by 1, 2 or 3 groups selected from linear or branched (C₁-C₆)alkyl, linear or branched halo(C₁-C₆)alkyl, —Y₁—OR′, —Y—NR′R″, oxo, halogen, in which R′ and R″ independently of one another represent a hydrogen atom, a linear or branched (C₁-C₆)alkyl group, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by a linear or branched (C₁-C₆)alkyl group. Even more preferably. Cy₁ represents a phenyl group, a thienyl group, a thiazolyl group, an imidazolyl group, a pyridinyl group, a pyrimidinyl group, or a dihydrothienodioxinyl group, said groups are substituted by 1, 2 or 3 groups selected from linear or branched (C₁-C₆)alkyl, linear or branched halo((C₁-C₆)alkyl, —Y₁—OR′, —Y₁—NR′R″, oxo, halogen, in which R′ and R″ independently of one another represent a hydrogen atom, a linear or branched (C₁-C₆)alkyl group, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by a linear or branched (C₁-C₆)alkyl group. Advantageously, Cy₁ represents a phenyl group, a pyrrolyl group, a thienyl group, a thiazolyl group, an oxazolyl group, an imidazolyl group, a pyrazolyl group, a pyridinyl group, a pyrimidinyl group, a dihydrothienodioxinyl group, a cyclopropyl group, or a cyclobutyl group, said groups are substituted by 1, 2 or 3 groups selected from methyl, fluorine or chlorine. More advantageously, Cy₁ represents a phenyl group, a thienyl group, a thiazolyl group, an imidazolyl group, a pyridinyl group, a pyrimidinyl group, or a dihydrothienodioxinyl group, said groups are substituted by 1, 2 or 3 groups selected from methyl, fluorine or chlorine.

Advantageously, L represents a linear or branched (C₁-C₆)alkyl group, a -Cy₂ group, a —CH₂-Cy₂ group, or a —CF₂—Cy₂ group. More advantageously, L represents a -Cy₂ group. Preferably, Cy₂ represents a cyclobutyl group, a cyclopentyl group, a cyclohexyl group, a piperidinyl group, a tetrahydropyranyl group, a phenyl group, a pyrrolyl group, a thienyl group, a thiazolyl group, an oxazolyl group, an imidazolyl group, a pyridinyl group, a pyrazinyl group, a pyrimidinyl group, a pyridazinyl group, an indolyl group, a dihydroindolyl group, a isoquinolinyl group, a dihydrothienodioxinyl group, a benzothiazolyl group, or a quinazolinonyl group. More preferably, Cy₂ represents a cyclobutyl group, a cyclopentyl group, a cyclohexyl group, a tetrahydropyranyl group, a phenyl group, a pyrrolyl group, a thienyl group, a thiazolyl group, a pyridinyl group, a pyradinyl group, a pyrimidinyl group, a pyridazinyl group, an indolyl group, a isoquinolinyl group, or a quinazolinonyl group.

Preferably, Cy₂ represents a cycloalkyl group, a heterocycloalkyl group, an aryl group or a heteroaryl group which are substituted by 1, 2 or 3 groups selected from linear or branched (C₁-C₆)alkyl, linear or branched halo(C₁-C₆)alkyl, —Y₁—OR′, —Y₁—NR′R″, oxo, halogen, in which R′ and R″ independently of one another represent a hydrogen atom, a linear or branched (C₁-C₆)alkyl group, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by a linear or branched (C₁-C₆)alkyl group. More preferably, Cy₂ represents a cyclobutyl group, a cyclopentyl group, a cyclohexyl group, a piperidinyl group, a tetrahydropyranyl group, a phenyl group, a pyrrolyl group, a thienyl group, a thiazolyl group, an oxazolyl group, an imidazolyl group, a pyridinyl group, a pyrazinyl group, a pyrimidinyl group, a pyridazinyl group, an indolyl group, a dihydroindolyl group, a isoquinolinyl group, a dihydrothienodioxinyl group, a benzothiazolyl group, or a quinazolinonyl group, said groups are substituted by 1, 2 or 3 groups selected from linear or branched (C₁-C₆)alkyl, linear or branched halo(C₁-C₆)alkyl, —Y₁—OR′, —Y₁—NR′R″, oxo, halogen, in which R′ and R″ independently of one another represent a hydrogen atom, a linear or branched (C₁-C₆)alkyl group, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by a linear or branched (C₁-C₆)alkyl group. Even more preferably, Cy₂ represents cyclobutyl group, a cyclopentyl group, a cyclohexyl group, a tetrahydropyranyl group, a phenyl group, a pyrrolyl group, a thienyl group, a thiazolyl group, a pyridinyl group, a pyrazinyl group, a pyrimidinyl group, a pyridazinyl group, an indolyl group, a isoquinolinyl group, or a quinazolinonyl group, said groups are substituted by 1, 2 or 3 groups selected from linear or branched (C₁-C₆)alkyl, linear or branched halo(C₁-C₆)alkyl, —Y₁—OR′, —Y₁—NR′R″, oxo, halogen, in which R′ and R″ independently of one another represent a hydrogen atom, a linear or branched (C₁-C₆)alkyl group, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by a linear or branched (C₁-C₆)alkyl group. Advantageously, Cy₂ represents a cyclobutyl group, a cyclopentyl group, a cyclohexyl group, a piperidinyl group, a tetrahydropyranyl group, a phenyl group, a pyrrolyl group, a thienyl group, a thiazolyl group, an oxazolyl group, an imidazolyl group, a pyridinyl group, a pyrazinyl group, a pyrimidinyl group, a pyridazinyl group, an indolyl group, a dihydroindolyl group, a isoquinolinyl group, a dihydrothienodioxinyl group, a benzothiazolyl group, or a quinazolinonyl group, said groups are substituted by 1, 2 or 3 groups selected from fluorine, bromine, iodine, chlorine, methyl, trifluoromethyl, methoxy, ethoxy, oxo and —Y₁—NR′R″ wherein Y₁ is a bond and R′ and R″ represent a methyl group or the pair (R′, R″) together with the nitrogen atom to which they are attached forms a piperazinyl group, more preferably a 4-methyl-piperazinyl group, a morpholinyl group or a pyrrolidinyl group. More advantageously, Cy₂ represents cyclobutyl group, a cyclopentyl group, a cyclohexyl group, a tetrahydropyranyl group, a phenyl group, a pyrrolyl group, a thienyl group, a thiazolyl group, a pyridinyl group, a pyrazinyl group, a pyrimidinyl group, a pyridazinyl group, an indolyl group, a isoquinolinyl group, or a quinazolinonyl group, said groups are substituted by 1, 2 or 3 groups selected from fluorine, bromine, iodine, chlorine, methyl, trifluoromethyl, methoxy, ethoxy, oxo and —Y₁—NR′R″ wherein Y₁ is a bond and R′ and R″ represent a methyl group or the pair (R′, R″) together with the nitrogen atom to which they are attached forms a piperazinyl group, more preferably a 4-methyl-piperazinyl group, a morpholinyl group or a pyrrolidinyl group.

In some preferred embodiment of the invention, K represents a phenyl group, a pyrrolyl group, a thienyl group, a thiazolyl group, an oxazolyl group, an imidazolyl group, a pyrazolyl group, a pyridinyl group, a pyrimidinyl group, a dihydrothienodioxinyl group, a cyclopropyl group, or a cyclobutyl group and L represents a cyclobutyl group, a cyclopentyl group, a cyclohexyl group, a piperidinyl group, a tetrahydropyranyl group, a phenyl group, a benzyl group, a pyrrolyl group, a thienyl group, or a pyridinyl group. More preferably, K represents a phenyl group, a thienyl group, a thiazolyl group, an imidazolyl group, a pyridinyl group, a pyrimidinyl group or a dihydrothienodioxinyl group, and L represents a cyclobutyl group, a cyclopentyl group, a cyclohexyl group, a tetrahydropyranyl group, a phenyl group, a benzyl group, a pyrrolyl group, or a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group. Even more preferably, K represents a phenyl group, a thienyl group, a thiazolyl group, an imidazolyl group, a pyridinyl group, a pyrimidinyl group or a dihydrothienodioxinyl group, and L represents a cyclobutyl group, a cyclopentyl group, a cyclohexyl group, a tetrahydropyranyl group, a phenyl group, a benzyl group, a pyrrolyl group, or a pyridinyl group, said groups may be substituted by 1 or 2 groups selected from fluorine, chlorine, methyl, trifluoromethyl and —Y₁—NR′R″ wherein Y₁ is a bond and R′ and R″ represent a methyl group.

In a preferred embodiment. K represents a thienyl group and L represents a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group.

In another preferred embodiment, K represents a pyridinyl group and L represents a phenyl group, a pyrrolyl group or a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group.

In another preferred embodiment, K represents a pyrimidinyl group and L represents a phenyl group or a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group.

In another preferred embodiment, K represents a thiazolyl group and L represents a cyclobutyl group, a cyclopentyl group, a cyclohexyl group, a tetrahydropyranyl group, a phenyl group, a benzyl group, or a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group.

In another preferred embodiment, K represents an imidazolyl group and L represents a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group.

In another preferred embodiment, K represents a phenyl group and L represents a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group.

In another preferred embodiment, K represents a dihydrothienodioxinyl group and L represents a pyridinyl group, said groups may be substituted by 1 or 2 groups selected from linear or branched (C₁-C₆)alkyl, linear or branched halo(C₁-C₆)alkyl, —Y₁—NR′R″, halogen, in which R′ and R″ independently of one another represent a hydrogen atom or a linear or branched (C₁-C₆)alkyl group.

Other compounds of the invention to which preference is given are those wherein K represents a bond and L represents a phenyl group, a thienyl group, a thiazolyl group, an oxazolyl group, an imidazolyl group, a pyridinyl group, a pyrazinyl group, a pyrimidinyl group, a pyridazinyl group, an indolyl group, a dihydroindolyl group, a isoquinolinyl group, a dihydrothienodioxinyl group, a benzothiazolyl group, or a quinazolinonyl group.

More preferably. K represents a bond and L represents a phenyl group, a thienyl group, a thiazolyl group, a pyridinyl group, a pyrazinyl group, a pyrimidinyl group, a pyridazinyl group, an indolyl group, a isoquinolinyl group, or a quinazolinonyl group, each said group may be substituted by 1 or 2 groups selected from linear or branched (C₁-C₆)alkyl, linear or branched halo(C₁-C₆)alkyl, —Y₁—OR′, —Y₁—NR′R″, halogen, in which R′ and R″ independently of one another represent a linear or branched (C₁-C₆)alkyl group, or the pair (R′, R″) together with the nitrogen atom to which they are attached forms a non-aromatic ring composed of from 5 to 7 ring members, which may contain in addition to the nitrogen a second heteroatom selected from oxygen and nitrogen, it being understood that the second nitrogen in question may be substituted by a linear or branched (C₁-C₆)alkyl group. Even more preferably, K represents a bond and L represents a phenyl group, a thienyl group, a thiazolyl group, a pyridinyl group, a pyrazinyl group, a pyrimidinyl group, a pyridazinyl group, an indolyl group, a isoquinolinyl group, or a quinazolinonyl group, each said group may be substituted by 1 or 2 groups selected from fluorine, bromine, iodine, chlorine, methyl, trifluoromethyl, methoxy, ethoxy, oxo and —Y₁—NR′R″ wherein Y₁ is a bond and the pair (R′, R″) together with the nitrogen atom to which they are attached forms a piperazinyl group, more preferably a 4-methyl-piperazinyl group, a morpholinyl group or a pyrrolidinyl group.

In a preferred embodiment, the -J-K-L group linked to the piperidinyl ring is defined such as J represents a —C(O)— group. K represents a -Cy₁- group and L represents a -Cy₂ group.

In another preferred embodiment, the -J-K-L group is defined such as J represents a —CH₂— group, K represents a bond and L represents a -Cy₂ group.

In another preferred embodiment, the -J-K-L group is defined such as J represents a —C(O)— group, K represents a bond and L represents a -Cv₂ group.

In another preferred embodiment, the -J-K-L group is defined such as J represents a —CH₂— group, K represents a -Cy₁- group and L represents a -Cy₂ group.

In another preferred embodiment, the -J-K-L group is defined such as J represents a —C(O)NH— group. K represents a bond and L represents a -Cy₂ group.

In another preferred embodiment, the -J-K-L group is defined such as J represents a —C(O)NH— group, K represents a -Cy:— group and L represents a -Cy₂ group.

In another preferred embodiment, the -J-K-L group is defined such as J represents a —SO₂— group. K represents a bond and L represents a -Cy₁ group.

In a preferred embodiment of the invention, the present invention relates to compounds of formula (I-a):

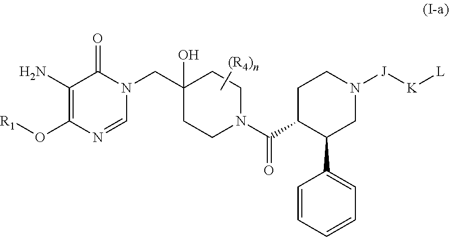

wherein R₁, R₄, J, K, L and n are as defined for formula (I).

In another preferred embodiment of the invention, the present invention relates to compounds of formula (I-b).

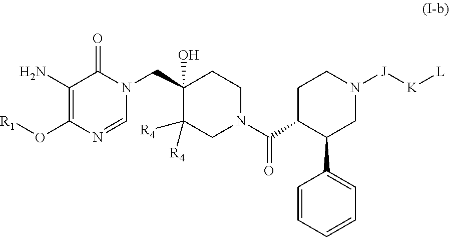

wherein R₁, J, K and L are as defined for Formula (I) and R₄ represents a halogen atom, more preferably, a fluorine atom.

Preferably, R₈ represents a linear or branched (C₁-C₆)alkyl group or a linear or branched halo(C₁-C₆)alkyl group. More preferably. R₈ represents a tert-butyl group or a 2,2,2-trifluoroethyl group.

Preferred compounds of the invention are:
5-amino-3-{[(4S)-3,3-difluoro-4-hydroxy-1-{(3R,4R)-1-](2-methylpyrimidin-4-yl)methyl]-3-phenylpiperidine-4-carbonyl}piperidine-4-yl]methyl}-6-(4-fluorophenoxy)pyrimidin-4(3H)-one;
5-amino-6-(4-chlorophenoxy)-3-[(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one;
5-amino-6-(3-chloro-5-methoxyphenoxy)-3-(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one;
5-amino-6-(4-chloro-3-fluorophenoxy)-3-[(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one;
5-amino-6-(4-fluoro-3-methoxyphenoxy)-3-[(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one;
5-amino-3-[(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]-6-[3-(trifluoromethyl)phenoxy]pyrimidin-4(3H)-one;
5-amino-3-[(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]-6-[3-(trifluoromethoxy)phenoxy]pyrimidin-4(3H)-one;
5-amino-3-(1-[(3R,4R)-1-(2-bromo-4-methyl-1,3-thiazole-5-carbonyl)-3-phenylpiperidine-4-carbonyl]-4-hydroxypiperidin-4-yl)methyl)-6-(4-fluorophenoxy)pyrimidin-4(3H)-one;
5-amino-3-({1-[(3R,4R)-1-(benzenesulfonyl)-3-phenylpiperidine-4-carbonyl]-4-hydroxypiperidin-4-yl]}methyl)-6-(4-fluorophenoxy)pyrimidin-4(3H-one;
5-amino-3-({1-[(3R,4R)-1-(3-bromobenzoyl)-3-phenylpiperidine-4-carbonyl]-4-hydroxypiperidin-4-yl}methyl)-6-(4-fluorophenoxy)pyrimidin-4(3H)-one;
5-amino-3-({1-[(3R,4R)-1-(5-bromopyridine-3-carbonyl)-3-phenylpiperidine-4-carbonyl]-4-hydroxypiperidin-4-yl}methyl)-6-(4-fluorophenoxy)pyrimidin-4(3H)-one;
5-amino-6-(4-fluorophenoxy)-3-({4-hydroxy-1-[(3R,4R)-1-(6′-methyl[3,3′-bipyridine)-5-carbonyl)-3-phenylpiperidine-4-carbonyl]piperidin-4-yl}methyl)pyrimidin-4(3H)-one;
5-amino-6-(4-fluorophenoxy)-3-({4-hydroxy-1-[(3R,4R)-3-phenyl-1-(5-phenylpyridine-3-carbonyl)piperidine-4-carbonyl]piperidin-4-yl}methyl)pyrimidin-4(3H)-one;
5-amino-3-({1-[(3R,4R)-1-benzoyl-3-phenylpiperidine-4-carbonyl]-4-hydroxypiperidin-4-yl}methyl)-6-(4-fluorophenoxy)pyrimidin-4(3H)-one;
5-amino-6-(4-fluorophenoxy)-3-({4-hydroxy-1-[(3R,4R)-1-(1-methyl-1H-indole-2-carbonyl)-3-phenylpiperidine-4-carbonyl(piperidin-4-yl}methyl)pyrimidin-4(3H)-one;
5-amino-3-({1-[(3R,4R)-1-(3-fluoro-5-iodothiophene-2-carbonyl)-3-phenylpiperidine-4-carbonyl]-4-hydroxypiperidin-4-yl)methyl)-6-(4-fluorophenoxy)pyrimidin-4(3H)-one;
5-amino-3-({1-((3R,4R)-1-[3-fluoro-5-(6-methylpyridin-3-yl)thiophene-2-carbonyl]-3-phenylpiperidine-4-carbonyl-4-hydroxypiperidin-4-yl)methyl]-6-(4-fluorophenoxy)pyrimidin-4(3H)-one;
5-amino-3-({(4S)-1-1 (3R,4R)-1-(2-bromo-4-methyl-1,3-thiazole-5-carbonyl)-3-phenylpiperidine-4-carbonyl]-3,3-difluoro-4-hydroxypiperidin-4-yl}methyl)-6-(4-fluorophenoxy)pyrimidin-4(3H)-one;
5-amino-6-(4-fluorophenoxy)-3-({4-hydroxy-1-[(3R,4R)-1-{4-methyl-2-[6-(trifluoromethyl)pyridin-3-yl]-1,3-thiazole-5-carbonyl-3-phenylpiperidine-4-carbonyl}piperidin-4-yl}methyl)pyrimidin-4(3H)-one;
5-amino-3-({1-(3R,4R)-1- (2-[6-(dimethylamino)pyridin-3-yl]-4-methyl-1,3-thiazole-5-carbonyl}-3-phenylpiperidine-4-carbonyl]-4-hydroxypiperidin-4-yl}methyl)-6-(4-fluorophenoxy)pyrimidin-4(3H)-one;
5-amino-3-{[(4S)-3,3-difluoro-1-[(3R,4R)-1-[2-(4-fluorophenyl)-4-methyl-1,3-thiazole-5-carbonyl-3-phenylpiperidine-4-carbonyl-4-hydroxypiperidin-4-yl]methyl}-6-(4-fluorophenoxy)pyrimidin-4(3H)-one;
5-amino-3-{[(4S)-3,3-difluoro-4-hydroxy-1- ((3R,4R)-1-[4-methyl-2-(6-methylpyridin-3-yl)-1,3-thiazole-5-carbonyl]-3-phenylpiperidine-4-carbonyl}piperidin-4-yl]methyl}-6-(4-fluorophenoxy)pyrimidin-4(3H)-one;
5-amino-3-[(4-hydroxy-1-[(3R,4R)-1-[4-methyl-2-(6-methylpyridin-3-yl)-1,3-thiazole-5-carbonyl]-3-phenylpiperidine-4-carbonyl}piperidin-4-yl)methyl]-6-phenoxypyrimidin-4(3H)-one;
5-amino-6-(4-fluorophenoxy)-3-[(4-hydroxy-1-[(3R,4R)-1-[4-methyl-2-(6-methylpyridin-3-yl)-1,3-thiazole-5-carbonyl]-3-phenylpiperidine-4-carbonyl}piperidin-4-yl)methyl]pyrindin-4(3H)-one;
5-amino-6-(4-fluorophenoxy)-3-[(1-{(3R,4R)-1-[2-(4-fluorophenyl)-4-methyl]-1,3-thiazole-5-carbonyl]-3-phenylpiperidine-4-carbonyl}-4-hydroxypiperidin-4-yl)methyl]pyrimidin-4(3H)-one;
5-amino-3-[[1-[(3R,4R)-1-[(2-bromothiazol-5-yl)methyl]-3-phenyl-piperidine-4-carbonyl]-4-hydroxy-4-piperidyl]methyl]-6-(4-fluorophenoxy)pyrimidin-4-one;
5-amino-6-(4-fluorophenoxy)-3-({4-hydroxy-1-(3R,4R)-1-{[2-(6-methylpyridin-3-yl)-1,3-thiazol-5-yl]methyl}-3-phenylpiperidine-4-carbonyl]piperidin-4-yl}methyl)pyrimidin-4(3H)-one
5-amino-6-(4-fluorophenoxy)-3-[(1-{(3R,4R)-1-[(2-fluorophenyl)methyl]-3-phenylpiperidine-4-carbonyl-4-hydroxypiperidin-4-yl)methyl]pyrimidin-4(3H)-one;
5-amino-3-({(4S)-3,3-difluoro-4-hydroxy-1-[(3R,4R)-1-([2-(6-methylpyridin-3-yl)-1,3-thiazol-5-yl]methyl-3-phenylpiperidine-4-carbonyl]piperidin-4-yl}methyl)-6-(4-fluorophenoxy)pyrimidin-4(3H)-one;
5-amino-3-{[(4S)-3,3-difluoro-1-[(3R,4R)-1-[(2-fluorophenyl)methyl]-3-phenylpiperidine-4-carbonyl-4-hydroxypiperidin-4-yl]methyl}-6-(4-fluorophenoxy)pyrimidin-4(3H)-one;
5-amino-3-({1-[(3R,4R)-1-(3-ethoxybenzoyl)-3-phenylpiperidine-4-carbonyl]-4-hydroxypiperidin-4-yl]methyl)-6-(4-fluorophenoxy)pyrimidin-4(3H)-one;
5-amino-3-({-[(3R,4R)-1-(2-benzyl-4-methyl-1,3-thiazole-5-carbonyl)-3-phenylpiperidine-4-carbonyl]-4-hydroxypiperidin-4-yl}methyl)-6-(4-fluorophenoxy)pyrimidin-4(3H)-one;
5-amino-6-(4-fluorophenoxy)-3-[(4-hydroxy-1-{(3R,4R)-3-phenyl-1-1-(pyridin-3-yl)methyl]piperidine-4-carbonyl}piperidin-4-yl)methyl]pyrimidin-4(3H)-one;
5-amino-6-(4-fluorophenoxy)-3-[(4-hydroxy-1-((3R,4R)-1-[4-methyl-2-(morpholin-4-yl)-1,3-thiazole-5-carbonyl]-3-phenylpiperidine-4-carbonyl}piperidin-4-yl)methyl]pyrimidin-4(3H)-one;
5-amino-6-(4-fluorophenoxy)-3-[(4-hydroxy-1-((3R,4R)-1-[4-methyl-2-(pyrrolidin-1-yl)-1,3-thiazole-5-carbonyl]-3-phenylpiperidine-4-carbonyl}piperidin-4-yl)methyl]pyrimidin-4(3H)-one;
5-amino-6-(4-fluorophenoxy)-3-({4-hydroxy-1-[(3R,4R)-1-(4-methyl-2-phenyl-1,3-thiazole-5-carbonyl)-3-phenylpiperidine-4-carbonyl]piperidin-4-yl}methyl)pyrimidin-4(3H)-one;
5-amino-6-(4-fluorophenoxy)-3-[(4-hydroxy-1-[(3R,4R)-1-[7-(6-methylpyridin-3-yl)-2,3-dihydrothieno[3,4-h][1,4]dioxine-5-carbonyl]-3-phenylpiperidine-4-carbonyl}piperidin-4-yl)methyl]pyrimidin-4(3H)-one;
(3R,4R)-4-(4-[[5-amino-4-(4-fluorophenoxy)-6-oxopyrimidin-[(6H)-yl]methyl}-4-hydroxypiperidine-1-carbonyl)-N-(4-methoxyphenyl)-3-phenylpiperidine-1-carboxamide;
(3R,4R)-4-(4-15-amino-4-(4-fluorophenoxy)-6-oxopyrimidin-[(6H)-yl]methyl]-4-hydroxypiperidine-1-carbonyl)-3-phenyl-N-[3-(trifluoromethyl)phenyl]piperidine-1-carboxamide;
(3R,4R)-4-(4-{[5-amino-4-(4-fluorophenoxy)-6-oxopyrimidin-[(6H)-yl]methyl}-4-hydroxypiperidine-1-carbonyl)-N-(3-bromophenyl)-3-phenylpiperidine-1-carboxamide;
(3R,4R)-4-[4-[[5-amino-4-(4-fluorophenoxy)-6-oxo-pyrimidin-1-yl]methyl]-4-hydroxy-piperidine-1-carbonyl]-N-[3-(6-methyl-3-pyrid yl)phenyl]-3-phenyl-piperidine-1-carboxamide;
5-amino-6-(4-fluorophenoxy)-3-({4-hydroxy-1-[(3R,4R)-1-(5-methylpyridine-3-carbonyl)-3-phenylpiperidine-4-carbonyl]piperidin-4-yl}methyl)pyrimidin-4(3H)-one;
5-amino-6-(4-fluorophenoxy)-3-[(4-hydroxy-1-[(3R,4R)-1-(I-methyl-2-(6-methylpyridin-3-yl)-1H-imidazole-5-carbonyl]-3-phenylpiperidine-4-carbonyl)piperidin-4-yl)methyl]pyrimidin-4(31)-one;
5-amino-3-(1-[(3R,4R)-1-(2,6-dimethylpyridine-4-carbonyl)-3-phenylpiperidine-4-carbonyl]-4-hydroxypiperidin-4-yl}methyl)-6-(4-fluorophenoxy)pyrimidin-4(3H)-one;
5-amino-3-({I-[(3R,4R)-1-(3-bromo-5-fluorobenzoyl)-3-phenylpiperidine-4-carbonyl]-4-hydroxypiperidin-4-yl)methyl)-6-(4-fluorophenoxy)pyrimidin-4(3H)-one;
5-amino-6-(4-fluorophenoxy)-3-(14-hydroxy-1-[(3R,4R)-1-(isoquinoline-5-carbonyl)-3-phenylpiperidine-4-carbonyl}piperidin-4-yl]methyl)pyrimidin-4(3H)-one;
5-amino-3-(1(4S)-3,3-difluoro-4-hydroxy-1-[(3R,4R)-1-[4-methyl-2-[6-(trifluoromethyl)pyridin-3-yl]-1,3-thiazole-5-carbonyl-3-phenylpiperidine-4-carbonyl[piperidin-4-yl}methyl)-6-(4-fluorophenoxy)pyrimidin-4(3H)-one;
5-amino-3-{[(4S)-3,3-difluoro-1-(3R,4R)-1-[3-fluoro-5-(6-methylpyridin-3-yl)thiophene-2-carbonyl]-3-phenylpiperidine-4-carbonyl-4-hydroxypiperidin-4-yl]methyl-6-(4-fluorophenoxy)pyrimidin-4(3H)-one;
5-amino-3-[(1-[(3R,4R)-1-[5-chloro-3-fluoro-4-(6-methylpyridin-3-yl)thiophene-2-carbonyl-3-phenylpiperidine-4-carbonyl}-4-hydroxypiperidin-4-yl)methyl]-6-(4-fluorophenoxy)pyrimidin-4(3H)-one;
5-amino-3-{1(4S)-3,3-difluoro-4-hydroxy-1-((3R,4R)-1-[4-methyl-2-(6-methylpyridin-3-yl)-1,3-thiazole-5-carbonyl]-3-phenylpiperidine-4-carbonyl}piperidin-4-yl]methyl]-6-phenoxypyrimidin-4(3H)-one;
5-amino-3-[(1-{(3R,4R)-1-[3-fluoro-5-(6-methylpyridin-3-yl)benzoyl]-3-phenylpiperidine-4-carbonyl}-4-hydroxypiperidin-4-yl)methyl]-6-(4-fluorophenoxy)pyrimidin-4(3)-one;
   6-[(3R,4R)-4-(4-{[5-amino-4-(4-fluorophenoxy)-6-oxopyrimidin-[(6H)-yl]methyl]-4-hydroxypiperidine-1-carbonyl)-3-phenylpiperidine-1-carbonyl]-3-methylquinazolin-4(3H)-one;
5-amino-3-[[4-hydroxy-1-[(3R,4R)-1-[4-methyl-2-[6-(trifluoromethyl)-3-pyridyl]thiazole-5-carbonyl]-3-phenyl-piperidine-4-carbonyl]-4-piperidyl]methyl]-6-[3-(trifluoromethyl)phenoxy]pyrimidin-4-one;
5-amino-3-[1-[(3R,4R)-1-[2-[6-(dimethylamino)-3-pyridyl]-4-methyl-thiazole-5-carbonyl]-3-phenyl-piperidine-4-carbonyl]-4-hydroxy-4-piperidyl]methyl]-6-[3-(trifluoromethyl)phenoxy]pyrimidin-4-one;
5-amino-3-[[1-[(3R,4R)-1-(3-chloro-1-methyl-indole-2-carbonyl)-3-phenyl-piperidine-4-carbonyl]-4-hydroxy-4-piperidyl]methyl]-6-(4-fluorophenoxy)pyrimidin-4-one;
5-amino-3-[[1-[(3R,4R)-1-(3,5-difluorobenzoyl)-3-phenyl-piperidine-4-carbonyl]-4-hydroxy-4-piperidyl]methy)]-6-(4-fluorophenoxy)pyrimidin-4-one;
5-amino-3-[[1-(3R,4R)-1-(3,5-dichlorobenzoyl)-3-phenyl-piperidine-4-carbonyl]-4-hydroxy-4-piperidyl]methyl]-6-(4-fluorophenoxy)pyrimidin-4-one;
5-amino-6-[4-(aminomethyl)phenoxy]-3-[[4-hydroxy-1-[(3R,4R)-1-[4-methyl-2-(6-methyl-3-pyridyl)thiazole-5-carbonyl]-3-phenyl-piperidine-4-carbonyl-4-piperidyl]methyl]pyrimidin-4-one;
5-amino-3-[[1-[(3R,4R)-1-(5-chloropyridine-3-carbonyl)-3-phenyl-piperidine-4-carbonyl]-4-hydroxy-4-piperidyl]methyl]-6-(4-fluorophenoxy)pyrimidin-4-one;
5-amino-6-(4-fluorophenoxy)-3-[[1-[(3R,4R)-1-(5-fluoropyridine-3-carbonyl)-3-phenyl-piperidine-4-carbonyl]-4-hydroxy-4-piperidyl]methyl]pyrimidin-4-one;
5-amino-6-(4-chloro-3-fluoro-phenoxy)-3-[[(4S)-3,3-difluoro-4-hydroxy-1-[(3R,4R)-1-[(5-methylpyrazin-2-yl)methyl]-3-phenyl-piperidine-4-carbonyl-4-piperidyl]methyl]pyrimidin-4-one;
5-amino-3-[[(4S)-3,3-difluoro-4-hydroxy-1-[(3R,4R)-1-(5-methylpyrazin-2-yl)methyl]-3-phenyl-piperidine-4-carbonyl]-4-piperidyl]methyl]-6-(4-fluoro-3-methoxy-phenoxy)pyrimidin-4-one;
5-amino-3-[[(4S)-3,3-difluoro-4-hydroxy-1-[(3R,4R)-1-(5-methylpyridine-3-carbonyl)-3-phenyl-piperidine-4-carbonyl]-4-piperidyl]methyl]-6-(4-fluorophenoxy)pyrimidin-4-one;
5-amino-3-[[1-(3R,4R)-1-(2-cyclohexyl-4-methyl-thiazole-5-carbonyl)-3-phenyl-piperidine-4-carbonyl-4-hydroxy-4-piperidyl]methyl}-6-(4-fluorophenoxy)pyrimidin-4-one;
5-amino-6-(4-fluorophenoxy)-3-[[4-hydroxy-1-[(3R,4R)-1-[3-(6-methyl-3-pyrid yl)phenyl]sulfonyl-3-phenyl-piperidine-4-carbonyl]-4-piperidyl]methyl]pyrimidin-4-one;
5-amino-3-[[(4S)-1-[(3R,4R)-1-[(2-chloropyrimidin-4-yl)methyl]-3-phenyl-piperidine-4-carbonyl]-3,3-difluoro 4-hydroxy-4-piperidyl]methyl]-6-(4-fluorophenoxy)pyrimidin-4-one;
5-amino-3-[[1-[(3R,4R)-1-(2-cyclopentyl-4-methyl-thiazole-5-carbonyl)-3-phenyl-piperidine-4-carbonyl]-4-hydroxy-4-piperidyl]methyl]-6-(4-fluorophenoxy)pyrimidin-4-one;
5-amino-3-[[1-[(3R,4R)-1-(3-chloro-5-methyl-benzoyl)-3-phenyl-piperidine-4-carbonyl]-4-hydroxy-4-piperidyl]methyl]-6-(4-fluorophenoxy)pyrimidin-4-one;
5-amino-6-(4-fluorophenoxy)-3-[[4-hydroxy-1-(3R,4R)-3-phenyl-1-(5-pyrrol-1-ylpyridine-3-carbonyl)piperidine-4-carbonyl]-4-piperidyl]methyl]pyrimidin-4-one;
5-amino-3-[[(4S)-3,3-difluoro-4-hydroxy-1-[(3R,4R)-1-[[6-(6-methyl-3-pyridyl)pyrimidin-4-yl]methyl]-3-phenyl-piperidine-4-carbonyl]-4-piperidyl]methyl]-6-(4-fluorophenoxy)pyrimidin-4-one;
5-amino-3-[[1-(3R,4R)-1-(2-cyclobutyl-4-methyl-thiazole-5-carbonyl)-3-phenyl-piperidine-4-carbonyl]-4-hydroxy-4-piperidyl]methyl]-6-(4-fluorophenoxy)pyrimidin-4-one;
5-amino-6-(4-chlorophenoxy)-3-[[(4S)-3,3-difluoro-4-hydroxy-1-[(3R,4R)-1-[(2-methylpyrimidin-4-yl)methyl]-3-phenyl-piperidine-4-carbonyl]-4-piperidyl]methyl]pyrimidin-4-one;
5-amino-3-[[4-hydroxy-1-[(3R,4R)-1-[4-methyl-2-(6-methyl-3-pyridyl)thiazole-5-carbonyl]-3-phenyl-piperidine-4-carbonyl]-4-piperidyl]methyl]-6-(3-pyridyloxy)pyrimidin-4-one;
5-amino-3-[[(4S)-3,3-difluoro-4-hydroxy-1-[(3R,4R)-1-[[2-(6-methyl-3-pyridyl)pyrimidin-4-yl]methyl]-3-phenyl-piperidine-4-carbonyl]-4-piperidyl]methyl]-6-(4-fluorophenoxy)pyrimidin-4-one;
5-amino-3-[[(4S)-3,3-difluoro-4-hydroxy-1-[(3R,4R)-3-phenyl-1-[(2-phenylpyrimidin-4-yl)methyl]piperidine-4-carbonyl]-4-piperidyl]methyl]-6-(4-fluorophenoxy)pyrimidin-4-one;
amino-3-[[(4S)-3,3-difluoro-4-hydroxy-1-(3R,4R)-1-[4-methyl-2-(6-methyl-3-pyridyl)thiazole-5-carbonyl]-3-phenyl-piperidine-4-carbonyl]-4-piperidyl]methyl]-6-(4-fluoro-3-methoxy-phenoxy)pyrimidin-4-one;
5-amino-6-(4-chloro-3-methoxy-phenoxy)-3-[[(4S)-3,3-difluoro-4-hydroxy-1-1(3R,4R)-1-[(2-methylpyrimidin-4-yl))methyl]-3-phenyl-piperidine-4-carbonyl]-4-piperidyl]methyl]pyrimidin-4-one;
5-amino-6-(4-chloro-3-methoxy-phenoxy)-3-(4S)-3,3-difluoro-4-hydroxy-1-1(3R,4R)-1-[(5-methylpyrazin-2-yl)methyl]-3-phenyl-piperidine-4-carbonyl]-4-piperidyl]methyl]pyrimidin-4-one;
5-amino-6-(4-chloro-3-methoxy-phenoxy)-3-[[(4S)-3,3-difluoro-4-hydroxy-1-(3R,4R)-1-[4-methyl-2-(6-methyl-3-pyridyl)thiazole-5-carbonyl]-3-phenyl-piperidine-4-carbonyl]-4-piperidyl]methyl]pyrimidin-4-one;
5-amino-3-[[(4S)-3,3-difluoro-4-hydroxy-1-[(3R,4R)-1-[4-methyl-2-[6-methyl-5-(trifluoromethyl)-3-pyridyl]thiazole-5-carbonyl]-3-phenyl-piperidine-4-carbonyl]-4-piperidyl]methyl]-6-(4-fluorophenoxy)pyrimidin-4-one;
5-amino-3-[[1-[(3R,4R)-1-[2-(3,3-difluorocyclobutyl)-4-methyl-thiazole-5-carbonyl-3-phenyl-piperidine-4-carbonyl-4-hydroxy-4-piperidyl]methyl]-6-(4-fluorophenoxy)pyrimidin-4-one;
5-amino-3-[[(4S)-1-[(3R,4R)-1-[2-(3,3-difluorocyclobutyl)-4-methyl-thiazole-5-carbonyl]-3-phenyl-piperidine-4-carbonyl]-3,3-difluoro-4-hydroxy-4-piperidyl]methyl]-6-(4-fluorophenoxy)pyrimidin-4-one;
5-amino-3-[[(4S)-3,3-difluoro-4-hydroxy-1-[(3R,4R)-1-(4-methyl-2-tetrahydropyran-4-yl-thiazole-5-carbonyl)-3-phenyl-piperidine-4-carbonyl]-4-piperidyl]methyl]-6-(4-fluorophenoxy)pyrimidin-4-one;
5-amino-3-[[((4S)-3,3-difluoro-4-hydroxy-1-[(3R,4R)-1-[(5-methylpyrazin-2-yl)methyl]-3-phenyl-piperidine-4-carbonyl]-4-piperidyl]methyl]-6-(4-fluorophenoxy)pyrimidin-4-one;
   5-amino-3-[[(4S)-3,3-difluoro-4-hydroxy-1-[(3R,4R)-3-phenyl-1-(pyrazin-2-ylmethyl)piperidine-4-carbonyl]-4-piperidyl]methyl]-6(4-fluorophenoxy)pyrimidin-4-one;
5-amino-3-[[(4S)-3,3-difluoro-4-hydroxy-1-[(3R,4R)-1-[(6-methylpyrazin-2-yl)methyl-3-phenyl-piperidine-4-carbonyl]-4-piperidyl]methy]-6-(4-fluorophenoxy)pyrimidin-4-one;
5-amino-6-[4-(aminomethyl)phenoxy]-3-[[(4S)-3,3-difluoro-4-hydroxy-1-(3R,4R)-1-[4-methyl-2-(6-methyl-3-pyridyl)thiazole-5-carbonyl]-3-phenyl-piperidine-4-carbonyl]-4-piperidyl]methyl]pyrimidin-4-one;
5-amino-3-[[(4S)-3,3-difluoro-4-hydroxy-1-[(3R,4R)-1-(3-methylpyrazin-2-yl)methyl]-3-phenyl-piperidine-4-carbonyl]-4-piperidyl]methyl]-6-(4-fluorophenoxy)pyrimidin-4-one;
5-amino-3-[[(4S)-3,3-difluoro-4-hydroxy-1-(3R,4R)-3-phenyl-1-(pyrimidin-4-ylmethyl)piperidine-4-carbonyl]-4-piperidyl]methyl]-6-(4-fluorophenoxy)pyrimidin-4-one;
5-amino-6-(4-chlorophen)yl)-3-[[(4S)-3,3-difluoro-4-hydroxy-1-[(3R,4R)-1-(5-methylpyrazin-2-yl)methyl]-3-phenyl-piperidine-4-carbonyl]-4-piperidyl]methyl]pyrindin-4-one;
5-amino-6-[4-(aminomethyl)phenoxy]-3-[(4S)-1-[(3R,4R)-1-(3,5-dichlorobenzoyl)-3-phenyl-piperidine-4-carbonyl]-3,3-difluoro-4-hydroxy-4-piperidyl]methyl]pyrimidin-4-one.

The invention relates also to a process for the preparation of compounds of formula (I), which process is characterized in that there is used as starting material the compound of formula (II):

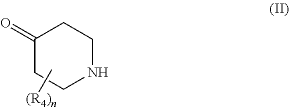

wherein R₄ and n are as defined for formula (I)
which is subjected to coupling with a compound of formula (III):

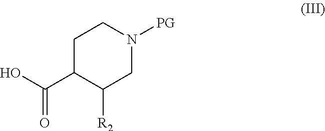

wherein R₂ is as defined for formula (i), and PG represents a protecting group of the amine function.
to yield the compound of formula (IV):

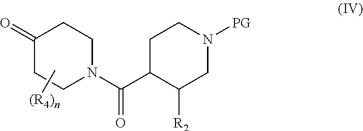

wherein R₂, R₄, n and PG are as defined hereinbefore.
compound of formula (IV) which is further converted to an epoxide compound of formula (V):

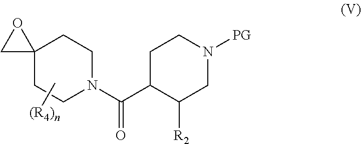

wherein R₂, R₄, n and PG are as defined hereinbefore,
compound of formula (V) which is further subjected to coupling with compound of formula (VI):

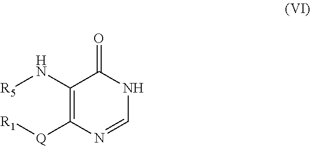

wherein R₁, R₅ and Q are as defined for formula (I),
to yield the compound of frenula (VII):

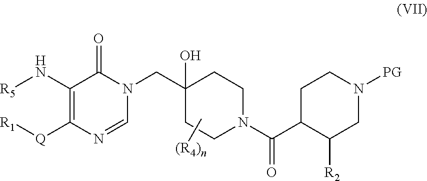

wherein R₁, R₂, R₄, R₅, Q, n and PG are as defined hereinbefore,
compound of formula (VII) which is subjected to a reaction removing the protecting group PG, to yield compound of formula (I-a), a particular case of compound of formula (I):

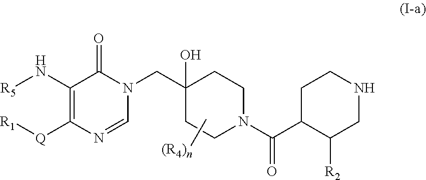

wherein R₁, R₂, R₄, R₅, Q and n are as defined hereinbefore,
compound a Formula (I-a), a particular case of compound of formula (I), which is further subjected to substitution reaction on piperidine's nitrogen to yield the compound of formula (I-b):

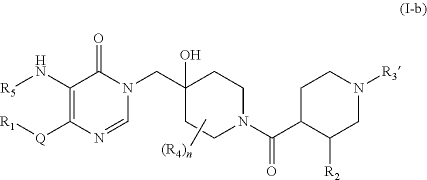

wherein R₁, R₂, R₄, R₅, Q and in are as defined hereinbefore and R₃′ represents a linear or branched (C₁-C₆)alkyl group, a linear or branched halo(C₁-C₆)alkyl, a linear or branched hydroxy(C₁-C₆)alkyl group, a —C(O)—R₈ group, a —C(O)—OR₈ group, a —C(O)—NH—R₈ group, or a

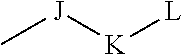

group.
compound of formula (I-a) and compound of formula (I-b), which constitute the totality of compounds of formula (I), may then be purified according to a conventional separation technique, which is converted, if desired, into its addition salts with a pharmaceutically acceptable acid or base and which is optionally separated into its isomers according to a conventional separation technique,
it being understood that at any moment considered appropriate during the course of the process described above, some groups hydroxy, amino, of the starting reagents or of the synthesis intermediates can be protected, subsequently deprotected and functionalized, as required by the synthesis.

In another embodiment of the invention, compounds of formula (I) may be obtained using an alternative process, which process is characterized in that there is used as starting material the compound of formula (VIII):

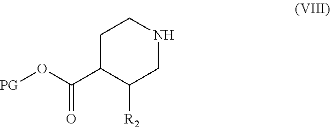

wherein R₂ is as defined for formula (I), and PG represents a protecting group of the carboxylic acid function,
which is subjected to substitution reaction on piperidine's nitrogen to yield the compound of formula (IX):

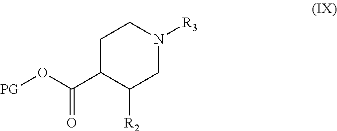

wherein R₂ and R₃ are as defined for formula (I), and PG represents a protecting group of the carboxylic acid function,
compound of formula (IX) which, after a reaction removing the protecting group PG, is further subjected to coupling with a compound of formula (II),
to yield the compound of Formula (X):

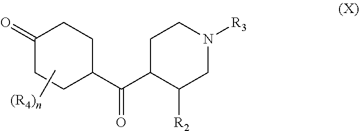

wherein R₂, R₃, R₄ and n are as defined hereinbefore,
compound of formula (X) which is further converted to an epoxide compound of formula (XI):

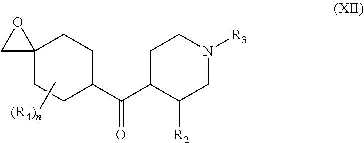

wherein R₂, R₃, R₄ and n are as defined hereinbefore.
compound of formula (XI) which is further subjected to coupling with compound of formula (VI):

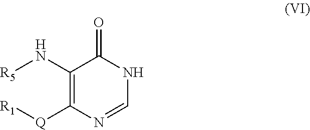

wherein R₁, R₅ and Q are as defined for formula (I),
to yield the compound of formula (I), which may be purified according to a conventional separation technique, which is convened, if desired, into its addition salts with a pharmaceutically acceptable acid or base and which is optionally separated into its isomers according to a conventional separation technique,
it being understood that at any moment considered appropriate during the course of the process described above, some groups (hydroxy, amino . . . ) of the starting reagents or of the synthesis intermediates can be protected, subsequently deprotected and functionalized, as required by the synthesis.

The compounds of formulae (II), (III), (VI) and (VIII) are either commercially available or can be obtained by the person skilled in the art using conventional chemical reactions described in the literature.

Pharmacological studies of the compounds of the invention have shown pro-apoptotic and/or anti-proliferative properties. The ability to reactivate the apoptotic process in cancerous cells is of major therapeutic interest in the treatment of cancers and of immune and auto-immune system diseases.

Among the cancer treatments envisaged there may be mentioned, without implying any limitation, treatment of cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas, ovarian cancer, non-small-cell lung cancer, prostate cancer, pancreatic cancer and small-cell lung cancer. More especially, the compounds according to the invention will be useful in the treatment of chemo-, targeted therapy- or radio-resistant cancers.

The present invention relates also to pharmaceutical compositions comprising at least one compound of formula (I) in combination with one or more pharmaceutically acceptable excipients. In particular, these pharmaceutical compositions are interesting for use as pro-apoptotic and/or anti-proliferative agents, particularly, in the treatment of cancers and of auto-immune and immune system diseases. Preferably, these pharmaceutical compositions can be used in the treatment of cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas, ovarian cancer, non-small-cell lung cancer, prostate cancer, pancreatic cancer and small-cell lung cancer.

Among the pharmaceutical compositions according to the invention there may be mentioned more especially those that are suitable for oral, parenteral, nasal, per- or trans-cutaneous, rectal, perlingual, ocular or respiratory administration, especially tablets or dragees, sublingual tablets, sachets, paquets, capsules, glossettes, lozenges, suppositories, creams, ointments, dermal gels, and drinkable or injectable ampoules.

The pharmaceutical compositions according to the invention comprise one or more excipients or carriers selected from diluents, lubricants, binders, disintegration agents, stabilisers, preservatives, absorbents, colorants, sweeteners, flavourings etc.

By Way of Non-Limiting Example there May be Mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: magnesium aluminium silicate, starch, gelatin, tragacanth, methylcellulose, sodium carboxymethylcellulose and polyvinylpyrrolidone,
as disintegrants: agar, alginic acid and its sodium salt, effervescent mixtures.

The dosage varies according to the sex, age and weight of the patient, the administration route, the nature of the therapeutic indication, or of any associated treatments, and ranges from 0.01 mg to 1 g per 24 hours in one or more administrations.

Furthermore, the present invention relates also to the combination of a compound of formula (I) with anti-cancer agents selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies, and also to pharmaceutical compositions comprising that type of combination and their use in the manufacture of medicaments for use in the treatment of cancer, particularly, cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas, ovarian cancer, non-small-cell lung cancer, prostate cancer, pancreatic cancer and small-cell lung cancer.

The combination of a compound of formula (I) with an anticancer agent may be administered simultaneously or sequentially. The administration route is preferably the oral route, and the corresponding pharmaceutical compositions may allow the instantaneous or delayed release of the active ingredients. The compounds of the combination may moreover be administered in the form of two separate pharmaceutical compositions, each containing one of the active ingredients, or in the form of a single pharmaceutical composition, in which the active ingredients are in admixture.

The compounds of formula (I) may also be used in combination with radiotherapy in the treatment of cancer.

The following Preparations and Examples illustrate the invention but do not limit it in any way.

General Procedures

All reagents obtained from commercial sources were used without further purification. Anhydrous solvents were obtained from commercial sources and used without further drying.

Flash chromatography was performed on ISCO CombiFlash Rf 200i with pre-packed silica-gel cartridges (RediSep®R_{f} Gold High Performance).

Thin layer chromatography was conducted with 5×10 cm plates coated with Merck Type 60 F₂₅₄ silica-gel.

Microwave heating was performed in an Anton Parr MonoWave or CEM Discover® instrument.

Preparative HPLC purifications were performed on an HANBON NP7000 Liquid Chromatography system with a Gemini-NX® 5 μM C18, 250 mm×50 mm i.d. column running at a flow rate of 99.9 mL min⁻¹ with UV diode array detection (210-400 nm) using 5 mM aqueous NH₄HCO₃ solution and MeCN as eluents unless specified otherwise.

Analytical LC-MS: The compounds of the present invention were characterized by high performance liquid chromatography-mass spectroscopy (HPLC-MS) on Agilent HP1200 with Agilent 6140 quadrupole LC/MS, operating in positive or negative ion electrospray ionization mode. Molecular weight scan range is 100 to 1350. Parallel UV detection was done at 210 nm and 254 nm. Samples were supplied as a 1 mM solution in MeCN, or in THF/H₂O (1:1) with 5 μL loop injection. LCMS analyses were performed on two instruments, one of which was operated with basic, and the other with acidic eluents.

Basic LCMS: Gemini-NX, 3 μm, C18, 50 mm×300 mm i.d. column at 23° C., at a flow rate of 1 mL·min⁻¹ using 5 mM ammonium bicarbonate (Solvent A) and acetonitrile (Solvent B) with a gradient starting from 100% Solvent A and finishing at 100% Solvent B over various/certain duration of time.

Acidic LCMS: ZORBAX Eclipse XDB-C18, 1.8 μm, 50 mm×4.6 mm i.d. column at 40° C. at a flow rate of 1 mL·min⁻¹ using 0.02% v/v aqueous formic acid (Solvent A) and 0.02% v/v formic acid in acetonitrile (Solvent B) with a gradient starting from 100% Solvent A and finishing at 100% Solvent B over various/certain duration of time.

¹H-NMR measurements were performed on Bruker Avance III 500 MHz spectrometer and Bruker Avance III 400 MHz spectrometer, using DMSO-d₆ or CDCl₃ as solvent, ¹H NMR data is in the form of delta values, given in part per million (ppm), using the residual peak of the solvent (2.50 ppm for DMSO-d₆ and 7.26 ppm for CDCl₃) as internal standard. Splitting patterns are designated as: s (singlet), d (doublet), t (triplet), q (quartet), quint (quintet), sept (septet), m (multiplet), br. (broad signal), brs (broad singlet), brd (broad doublet), brt (broad triplet), brq (broad quartet), brm (broad multiplet), vbrs (very broad singlet), dd (doublet of doublets), td (triplet of doublets), di (doublet of triplets), dq (doublet of quartet), ddd (doublet of doublet of doublets), dm (doublet of multiplets), tm (triplet of multiplets), qm (quartet of multiplets).

Combination gas chromatography and low resolution mass spectrometry were performed on Agilent 6850 gas chromatograph and Agilent 5975C mass spectrometer using 15 m×0.25 mm column with 0.25 μm HP-5MS coating and helium as carrier gas. Ion source: EI′, 70 eV, 230° C., quadrupole: 150° C., interface: 300° C.

High resolution mass spectrometry was performed on JEOL AccuTOF MS instrument connected to Agilent 7693A gas chromatograph on Rxi-5Sil MS column 15 m×0.25 mm column and helium was used as carrier gas. Ion source: E1+, 70 eV, 200° C., interface: 250° C.

HRMS were determined on a Shimadtu IT-TOF, ion source temperature 200° C., ESI +/−, ionization voltage: (+−) 4.5 kV. Mass resolution min, 10000.

Elementary analyses were performed on a Thermo Flash EA 1112 Elemental Analyzer.

IUPAC chemical names were generated using ACD/Name 2015 Pack 2 (File Version N₂₀E41, Build 75170, 19 Dec. 2014) or using ‘Structure to Name’ functionality within Accelrys Draw 4.2.

### List of Abbreviations

| Abbreviation | Name |
|---|---|
| abs. | absolute |
| aq. | aqueous |
| AtaPhos*PdCl₂ | bis(di-tert-butyl(4-dimethylaminophenyl)phosphine) |
| | dichloropalladium(II) |
| Boc | tert-butoxycarbonyl |
| cc. | concentrated |
| DCM | dichloromethane |
| DEE | diethyl ether |
| disp. | dispersion |
| DMF | dimethylformamide |
| EDC•HCl | N-(3-dimethylaminopropyl)-N′-ethylcarbodiimide |
| | hydrochloride |
| eq. | equivalent |
| LC | liquid chromatography |
| MeCN | acetonitrile |
| Me-THF | 2-methyltetrahydrofuran |
| MSM | methylsulfinylmethane |
| MTBE | tert-butyl methylether |
| r.t. | room temperature |
| sat. | saturated |
| THF | tetrahydrofuran |
| TMP•MgCl•LiCl | 2,2,6,6-tetramethylpiperidine-magnesium |
| | chloride-lithium chloride (1:1) complex |

General Procedure 1

4-chloro-6-methoxy-5-nitro-pyrimidine (1.0 eq.), the appropriate phenol (1.2 eq.), and potassium carbonate (1.2 eq.) were dissolved in MeCN. It was stirred at 80° C. till completion, then water was added to the reaction mixture. MeCN was evaporated. The residue extracted with DCM. The combined organic phase was dried over MgSO₄ and evaporated under reduced pressure to give Preparation R2a-R2n.

General Procedure 2

Autoclave was charged with Preparation R2a-R2h and R2k-R2n (1.0 eq.), Raney-nickel catalyst (10 w/w %) and 1,4-dioxane and then placed under a nitrogen atmosphere. After that it was filled with 10 bar 15 Hz gas. The reaction mixture was stirred in autoclave at r.t for 20 hours. The reaction mixture was removed from the autoclave and filtered. The filtrate was purified by preparative LC (on C-18 Gemini-NX 5 μm column, 5 mM aqueous NH₄HCO₃-MeCN, gradient). Solvent was evaporated under reduced pressure to give Preparation R3a-R3h and R3k-R3n.

General Procedure 3

Preparation R3a-R3n (1.0 eq.) was dissolved in 1,4-dioxane, then 1N hydrochloric acid (3.0-5.0 eq.) was added. It was stirred at 95° C. till completion, then the reaction mixture concentrated under reduced pressure to give Preparation R4a-R4n.

General Procedure 4

Preparation R4a-R4n (0.0 eq.), the appropriate epoxide derivative (1.0 eq.) and potassium carbonate (3.0 eq.) were dissolved in DMF. It was stirred at 70° C. till completion. The reaction mixture was directly injected through syringe filter to preparative HPLC (on C-18 Gemini-NX 5 μm column, 5 mM NH₄HCO₃ aqueous solution-MeCN, gradient 5-90%). Fractions were collected and concentrated under reduced pressure, then dried in vacuum at 50° C. for overnight.

General Procedure 5: Boc Deprotection

The appropriate Boc protected amine (1.0 eq.) was dissolved in 1,4-dioxane and 1N hydrochloric acid solution (5.0 eq.) was added. It was stirred at 70° C. till completion, then the solvents were evaporated under reduced pressure to give the appropriate amine derivative.

General Procedure 6: Acylation

The appropriate amine (1.0 eq.), EDC.HCl (3.0 eq.) and corresponding carboxylic acid (1.0 eq.) were stirred in pyridine at r.t. for 2-24 hours.

Work-Up 1:

The reaction mixture was evaporated, the residue was taken in DMF and injected to preparative LC (on C-18 Gemini-NX 5 μm column, 5 mM aqueous NH₄HCO₃-MeCN, gradient). The solvents were evaporated under reduced pressure.

Work-Up 2:

The reaction mixture was evaporated. The residue was triturated with water and the resulted solid was filtered off.

General Procedure 7: Urea Preparation from Isocyanate

The appropriate amine (1.0 eq.) and isocyanato derivative (1.3 eq.) were dissolved in DCM and stirred at rt, till completion. Then the solvent was evaporated under reduced pressure, dissolved in DMF and injected to preparative LC (on C-18 Gemini-NX 5 μm column, 5 mM aqueous NH₄HCOs-MeCN, gradient). The solvent was evaporated under reduced pressure to give the appropriate urea derivative.

General Procedure 8: Urea Preparation from Amine

Bis(trichloromethyl) carbonate (0.5 eq.) was dissolved in MeCN, then the appropriate alkyl/aryl amine (1.5 eq.) was added and stirred for 10 minutes, after then N,N-diethylethanamine (5.0 eq.) was added and the mixture was stirred for 1 h. Then amino-pyrimidone derivative (150 mg, 0.2876 mmol, 1.0 eq.) was added in 2 ml MeCN to the mixture and stirred at r.t. till completion. Then 6N NH₃ solution in methanol was added and the mixture was evaporated under reduced pressure, dissolved in DMF/methanol then it was injected to preparative LC (on C-18 Gemini-NX 5 μm column, 5 mM aqueous NH₄HCO₃-MeCN, gradient). The solvent was evaporated under reduced pressure to give the appropriate urea derivative.

General Procedure 9: Alkylation/Sulfonylation

The appropriate amine (1.0 eq.), corresponding sulfonyl chloride/alkyl halide (1.3 eq.) and K₂CO₃ (3.0 eq.) were stirred in DMF at r.t. till completion. The mixture was evaporated under reduced pressure, dissolved in DMF/methanol then it was injected to preparative LC (on C-18 Gemini-NX 5 μm column, 5 mM aqueous NH₄HCO₃-MeCN, gradient). Solvent was evaporated under reduced pressure.

General Procedure 10: Reductive Amination

The appropriate amine (100 mg, 0.1797 mmol), aldehyde derivative (1.3 eq.), sodium triacetoxyborohydride (5.0 eq.), acetic acid (5.0 eq.) were dissolved in THF and stirred at r.t. till completion. The reaction mixture was purified by preparative LC (on C-18 Gemini-NX 5 μm column, 5 mM aqueous NH₄HCO₃-MeCN, gradient). Solvent was evaporated under reduced pressure.

General Procedure 11

The corresponding halogenated component (0.0 eq.), corresponding boronic acid (2.5 eq.), ATAphos*PdCl₂ (0.1 eq.), Cs₂CO₃ (3.5 eq.) was diluted with THF and water (1:1). The mixture was flushed with nitrogen and microwaved at 80° C. for 100-150 minutes. The reaction mixture was injected through syringe filter to preparative LC (on C-18 Gemini-NX 5 μm column, 5 mM aqueous NH₄HCO₃-MeCN, gradient). Solvent was evaporated under reduced pressure.

### Preparation R2a: 4-(4-chlorophenoxy)-6-methoxy-5-nitro-pyrimidine

Using General Procedure 1 and 4-chlorophenol as reagent. Preparation R2a was obtained.

HRMS calculated for C₁₁H₈ClN₃O₄: 281.0203; found 281.0198 (M⁺ form).

¹H-NMR (400 MHz, dmso-d6) δ ppm 8.6 (s, 1H), 7.54 (m, 2H), 7.34 (m, 2H), 4.1 (s, 3H).

¹³C-NMR (100 MHz, dmso-d6) δ ppm 162.5, 161, 158.7, 150.7, 131, 130.3, 124.1, 56.7.

### Preparation R2b: 4-(3-chloro-5-methoxy-phenoxy)-6-methoxy-S-nitro-pyrimidine

Using General Procedure 1 and 3-chloro-5-methoxy-phenol as reagent, Preparation R2b was obtained. HRMS calculated for C₁₂H₁₀ClN₃O₅: 311.0309; found 312.0377 ((M+H)⁺ form).

¹H-NMR (500 MHz, dmso-d6) δ ppm 8.63 (s, 1H), 7.05 (t, 1H), 7.01 (t, 1H), 6.93 (t, 1H), 4.11 (s, 3H), 3.78 (s, 3H).

¹³C-NMR (125 MHz, dmso-d6) δ ppm 162.5, 161.4, 160.9, 158.7, 153.3, 134.6, 114.7, 112.9, 107.6, 56.7, 56.5.

### Preparation R2c: 4-(4-chloro-3-fluoro-phenoxy)-6-methoxy-5-nitro-pyrimidine

Using General Procedure 1 and 4-chloro-3-fluoro-phenol as reagent, Preparation R2c was obtained. HRMS calculated for C₁₁H₇ClFN₃O₄: 299.0109; found 300.0179 ((M+H)⁺ form).

¹H-NMR (500 MHz, dmso-d6) δ ppm 7.72 (t, 1H), 7.6 (dd, 1H), 7.24 (dm, 1H), 6.83 (s, 1H), 4.11 (s, 3H)

¹³C-NMR (125 MHz, dmso-d6) δ ppm 162.5, 160.7, 158.7, 157.7, 151.3, 131.6, 120.9, 119.9, 117.8, 112.1, 56.8

### Preparation R2d: 4-(4-fluoro-3-methoxy-phenoxy)-6-methoxy-5-nitro-pyrimidine

Using General Procedure 1 and 4-fluoro-3-methoxy-phenol as reagent, Preparation R2d was obtained. HRMS calculated for C₁₂H₁₀FN₃O₅: 295.0605; found 296.0675 ((M+H)⁺ form).

¹H-NMR (500 MHz, dmso-d6) δ ppm 8.84 (s, 1H), 7.33 (dd, 1H), 7.26 (dd, 1H), 6.91 (ddd, 1H), 3.81 (s, 3H), 3.81 (s, 3H).

¹³C-NMR (125 MHz, dmso-d6) δ ppm 159.1, 116.8, 113.8, 108.5, 56.9, 56.9.

### Preparation R2e: 4-methoxy-5-nitro-6-[3-(trifluoromethyl)phenoxy]pyrimidine

Using General Procedure 1 and 3-(trifluoromethyl)phenol as reagent, Preparation R2e was obtained. HRMS calculated for C₁₂H₈FN₃O₄: 315.0467; found 316.0545 ((M+H)⁺ form).

¹H-NMR (500 MH, dmso-d6) δ ppm 8.62 (s, 1H), 7.79 (m, 1H), 7.72 (m, 1H), 7.72 (m, 1H), 7.65 (m, 1H), 4.12 (s, 3H).

¹³C-NMR (125 MHz, dmso-d6) δ ppm 162.5, 160.9, 158.7, 152.2, 131.7, 131, 126.6, 124, 123.7, 119.5, 56.7.

### Preparation R2f: 4-methoxy-5-nitro-6-[3-(trifluoromethoxy)phenoxy]pyrimidine

Using General Procedure 1 and 3-(trifluoromethoxy)phenol as reagent, Preparation R2f was obtained. HRMS calculated for C₁₂H₈F₃N₃O₅: 331.0416; found 332.0488 ((M+H)⁺ form).

¹H-NMR (500 MHz, dmso-d6) 3 ppm 8.62 (s, 1H), 7.62 (t, 1H), 7.47 (s, 1H), 7.37 (m, 1H), 7.36 (m, 1H), 4.11 (s, 3H).

¹³C-NMR (125 MHz, dmso-d6) δ ppm 162.5, 160.8, 158.7, 152.6, 149.2, 131, 8, 121, 5, 121.4, 120.1, 119.4, 115.8, 56.7.

### Preparation R2g: 4-methoxy-5-nitro-6-phenoxy-pyrimidine

Using General Procedure 1 and phenol as reagent, Preparation R2g was obtained. HRMS calculated for C₁₁H₉N₃O₄: 247.0593; found 248.0672 ((M+H)⁺ form).

¹H-NMR (500 MHz, dmso-d6) δ ppm 8.59 (s, 1H), 7.48 (m, 2H), 7.33 (tin, 1H), 7.27 (dm, 2H), 4.1 (s, 3H).

¹³C-NMR (125 MHz, dmso-d6) δ ppm 162.4, 161.1, 158.7, 152, 130.4, 126.8, 122.1, 56.6.

### Preparation R2h: 4-(4-fluorophenoxy)-6-methoxy-5-nitro-pyrimidine

Using General Procedure 1 and 4-fluorophenol as reagent, Preparation R2h was obtained, HRMS calculated for C₁₁H₈FN₃O₄: 265.0499; found 265.0496 (M⁺ form).

¹H-NMR (400 MHz, dmso-d6) δ ppm 8.59 (s, 1H), 7.33 (m, 2H), 7.33 (m, 2H), 4.1 (s, 3H).

¹³C-NMR (10) MHz, dmso-d6) δ ppm 162.4, 161.6, 158.6, 124.1, 117, 56.7.

### Preparation R2j: 4-(6-methoxy-5-nitro-pyrimidin-4-yl)oxybenzonitrile

Using General Procedure 1 starting from 4-hydroxybenzonitrile as reagent, Preparation R2j was obtained. HRMS calculated for C₁₂H₈N₄O₄: 272.0546: found 273,0621 ((M+H)⁺ form).

¹H-NMR (400 MHz, dmso-d6) δ ppm 8.63 (s, 1H), 7.99 (d, 2H), 7.55 (d, 2H), 4.12 (s, 3H)

¹³C-NMR (100 MHz, dmso-d6) δ ppm 162.6, 160.6, 158.7, 155.4, 134.9, 123.5, 18.8, 109.9, 56.8

### Preparation R2k: 4-methoxy-5-nitro-6-(3-pyridyloxy)pyrimidine

Using General Procedure 1 and pyridin-3-ol as reagent, Preparation R2k was obtained. HRMS calculated for C₁₀H₈N₄O₄: 248,0546; found 249,0615 ((M+H)⁺ form).

¹H-NMR (500 MHz, dmso-d6) δ ppm 8.61 (s, 1H), 8.58 (d, 1H), 8.54 (dd, 1H), 7.82 (ddd, 1H), 7.55 (dd, 1H), 4.12 (s, 3H)

¹³C-NMR (125 MHz, dmso-d6) δ ppm 162.5, 160.9, 158.7, 148.8, 147.9, 143.8, 130.2, 125.1, 120.9, 56.7

### Preparation R2l: 4-(6-methoxy-5-nitro-pyrimidin-4-yl)oxybenzaldehyde

Using General Procedure 1 and 4-hydroxybenzaldehyde as reagent, Preparation R2l was obtained. HRMS calculated for C₁₂H₉N₃O₅: 275.0542; found 276,0612 ((M+H)⁺ form).

¹H-NMR (500 MHz, dmso-d6) δ ppm 10.03 (s, 1H), 8.63 (s, 1H), 8.03 (dm, 2H), 7.53 (dm, 2H), 4.12 (s, 3H)

¹³C-NMR (125 MHz, dmso-d6) δ ppm 192.4, 162.6, 160.7, 158.7, 156.4, 134.7, 131.9, 122.9, 121.2, 56.7

### Preparation R2m: 4-(4-chloro-3-methoxy-phenoxy)-6-methoxy-S-nitro-pyrimidine

Using General Procedure 1 and 4-chloro-3-methoxy-phenol as reagent, Preparation R2m was obtained. HRMS calculated for C₁₂H₁₀ClN₃O₅: 311.0309; found 312.0385 ((M+H)⁺ form)

¹H-NMR (400 MHz, dmso-d6) δ ppm 8.61 (s, 1H), 7.51 (d, 1H), 7.17 (d, 1H), 6.9 (dd, 1H), 4.11 (s, 3H), 3.83 (s, 3H)

¹³C-NMR (100 MHz, dmso-d6) δ ppm 162.4, 161, 158.7, 155.8, 151.6, 130.7, 119, 114.9, 107.7, 57, 56.7

### Preparation R2n: 4-(4-cyclopropyl-3-methoxy-phenoxy)-6-methoxy-5-nitro-pyrimidine

Using General Procedure 1 and 4-cyclopropyl-3-methoxy-phenol as reagent, Preparation R2n was obtained. HRMS calculated for C₁₅H₁₅N₃O₅: 317.1012; found 318.10810 ((M+H)⁺ form)

¹H-NMR (500 MHz, dmso-d6) δ ppm 8.58 (s, 1H), 6.88 (d, 1H), 6.88 (d, 1H), 6.72 (dd, 1H), 4.1 (s, 3H), 3.78 (s, 3H), 2.07 (m, 1H), 0.88/0.62 (m+m, 4H)

¹³C-NMR (125 MHz, dmso-d6) δ ppm 158.7, 125.5, 113.4, 105, 56.6, 56.2, 9.5, 8.2

### Preparation R3a: 4-(4-chlorophenoxy)-6-methoxy-pyrimidin-5-amine

Using General Procedure 2 starting from Preparation R2a as reagent. Preparation R3a was obtained. FIRMS calculated for C₁₁H₁₀ClN₃O₂: 251.0462: found 252,0523 ((M+H)⁺ form).

¹H-NMR (400 MHz, dmso-d6) δ ppm 7.76 (s, 1H), 7.45 (m, 2H), 7.18 (m, 2H), 4.9 (s, 2H), 3.95 (s, 3H).

¹³C-NMR (100 MHz, dmso-d6) δ ppm 158.1, 154.3, 153, 142.8, 129.8, 128.8, 123.3, 117.4, 54.4.

### Preparation R3b: 4-(3-chloro-5-methoxy-phenoxy)-6-methoxy-pyrimidin-5-amine

Using General Procedure 2 starting from Preparation R2b as reagent, Preparation R3b was obtained. HRMS calculated for C₁₂H₁₂ClN₃O₃: 281.0567: found 282.0645 ((M+H)⁺ form).

¹H-NMR (500 MHz, dmso-d6) δ ppm 7.79 (s, 1H), 6.87 (t, 1H), 6.81 (t, 1H), 6.73 (t, 1H), 4.91 (s, 2H), 3.96 (s, 3H), 3.77 (s, 3H).

¹³C-NMR (125 MHz, dmso-d6) δ ppm 161.2, 158.3, 155.8, 153.8, 142.8, 134.3, 117.8, 113.6, 110.7, 106.5, 56.3, 54.5.

### Preparation R3c: 4-(4-chloro-3-fluoro-phenoxy)-6-methoxy-pyrimidin-5-amine

Using General Procedure 2 starting from Preparation R2e as reagent, Preparation R3c was obtained. HRMS calculated for C₁₁H₉ClFN₃O₂: 269.0367: round 270.044 ((M+H)⁺ form).

¹H-NMR (400 MHz, dmso-d6) δ ppm 7.79 (s, 1H), 7.61 (t, 1H), 7.36 (dd, 1H), 7.06 (dm, 1H), 4.97 (br., 2H), 3.96 (s, 3H).

¹³C-NMR (100 MHz, dmso-d6) δ ppm 158.3, 157.7, 153.9, 153.7, 142.7, 131.2, 118.7, 117.7, 115.4, 110.7, 54.5.

### Preparation R3d: 4-(4-fluoro-3-methoxy-phenoxy)-6-methoxy-pyrimidin-5-amine

Using General Procedure 2 starting from Preparation R2d as reagent, Preparation R3d was obtained. HRMS calculated for C₁₂H₁₂FN₃O₃: 265.0863: found 266.0931 ((M+H)⁺ form).

¹H-NMR (500 MHz, dmso-d6) δ ppm 7.76 (s, 1H), 7.21 (dd, 1H), 7 (dd, 1H), 6.69 (dm, 1H), 4.85 (s, 2H), 3.95 (s, 3H), 3.8 (s, 3H).

¹³C-NMR (125 MHz, dmso-d6) δ ppm 142.9, 116.2, 113.3, 108.1.

### Preparation R3e: 4-methoxy-6-[3-(trifluoromethyl)phenoxy]pyrimidin-5-amine

Using General Procedure 2 starting from Preparation R2e as reagent, Preparation R3e was obtained. HRMS calculated for C₁₂H₁₀FN₃O₂: 285.0725: found 286.0796 ((M+H)+ form).

¹H-NMR (50 MHz, dmso-d6) δ ppm 7.79 (s, 1H), 7.64 (brt, 1H), 7.57 (dm, 1H) 7.52 (m, 1H), 7.48 (dm, 1H), 4.97 (br., 2H), 3.97 (s, 3H).

¹³C-NMR (25 MHz, dmso-d6) δ ppm 158.3, 154.5, 153.9, 142.8, 131.3, 130.7, 125.5, 124.3, 121.5, 118.1, 54.5.

### Preparation R3f: 4-methoxy-6-[3-(trifluoromethoxy)phenoxy]pyrimidin-5-amine

Using General Procedure 2 starting from Preparation R2f as reagent, Preparation R3f was obtained, HRMS calculated for C₁₂H₁₀N₃O₃: 301.0674; found 302.0742 ((M+H)⁺ form).

¹H-NMR (500 MHz, dmso-d6) δ ppm (m, 3H), 7.79 (s, 1H), 7.53 (m, 1H), 4.96 (br., 2H), 3.96 (s, 3H).

¹³C-NMR (125 MHz, dmso-d6) δ ppm 158.3, 1538, 152.2, 149.2, 142.8, 131.3, 120.5, 120.4/1117.2/114.4, 117.8, 54.5,

### Preparation R3e: 4-methoxy-6-phenoxy-pyrimidin-5-amine

Using General Procedure 2 starting from Preparation R2g as reagent, Preparation R3g was obtained. HRMS calculated for C₁₁H₁₁N₃O₂: 217.0851: found 218.092 ((M+H)⁺ form).

¹H-NMR (500 MHz, dmso-d6) δ ppm 7.75 (s, 1H), 7.4 (m, 2H), 7.19 (m, 1H), 7.12 (m, 2H), 4.85 (brs, 2H), 3.95 (s, 3H).

¹³C-NMR (125 MHz, dmso-d6) δ ppm 158, 154.7, 154.1, 142.9, 129.9, 124.9, 121.4, 117.3, 54.4.

### Preparation R3h: 4-(4-fluorophenoxy)-6-methoxy-pyrimidin-5-amine

Using General Procedure 2 starting from Preparation R2h as reagent, Preparation R3h was obtained. HRMS calculated for C₁₁H₁₀FN₃O₂: 235,0757; found 235.07503 (M⁺ form).

¹H-NMR (500 MHz, dmso-d6) δ ppm 7.74 (s, 1H), 7.23 (m, 2H), 7.18 (m, 2H), 4.86 (s, 2H), 3.95 (s, 3H).

¹³C-NMR (125 MHz, dmso-d6) δ ppm 159.2, 157.9, 154.9, 150.1, 142.9, 123.4, 116.4, 54.4.

### Preparation R3j: 4-(5-amino-6-methoxy-pyrimidin-4-yl)oxybenzonitrile

Preparation R2j (1.0 eq.), and tin(II) chloride dihydrate (3.5 eq.) were dissolved in 1,4-dioxane. The reaction mixture was stirred till completion at r.t. Then sat. NaHCO₃ solution and EtOAc were added. The suspension was filtered through Celite, washed with EtOAc and the layers were separated. The aqueous phase was extracted with EtOAc and Celite was washed again with DCM-MeOH. All organic phases were collected and concentrated under reduced pressure. The residue was purified by preparative HPLC (on C-18 Gemini-NX 5 μm column, 5 mM NH₄HCO₃ aqueous solution-MeCN, gradient 5-90%). Fractions were collected and concenrated under reduced pressure to give Preparation R3j. HRMS calculated for C₁₂H₁₀N₄O₂: 242.0804; found 243.088 ((M+H)⁺ form).

¹H-NMR (500 MHz, dmso-d6) δ ppm 7.88 (dm, 2H), 7.81 (s, 1H), 7.32 (dm, 2H), 5.04 (br., 2H), 3.97 (s, 3H).

¹³C-NMR (125 MHz, dmso-d6) δ ppm 158.6, 158.1, 152.9, 142.8, 134.5, 121.6, 119.1, 118.5, 107, 54.6.

### Preparation R3k: 4-methoxy-6-(3-pyridyloxy)pyrimidin-5-amine

Using General Procedure 2 starting from Preparation R2k as reagent, Preparation R3k was obtained. HRMS calculated for C₁₀H₈N₄O₄: 248.0546: found 249.0615 ((M+H)⁺ form).

¹H-NMR (500 MHz, dmso-d6) δ ppm 8.61 (s, 1H), 8.58 (d, 1H), 8.54 (dd, 1H), 7.82 (ddd, 1H), 7.55 (dd, 1H), 4.12 (s, 3H)

¹³C-NMR (125 MHz, dmso-d6) δ ppm 162.5, 160.9, 158.7, 148.8, 147.9, 143.8, 130.2, 125.1, 120.9, 56.7

### Preparation R3l: 4-(5-amino-6-methoxy-pyrimidin-4-yl)oxybenzaldehyde

Using General Procedure 2 starting from Preparation R21 as reagent. Preparation R3l was obtained. HRMS calculated for C₁₂H₁₁N₃O₃: 245.08; found 246,0873 ((M+H)⁺ form),

¹H-NMR (500 MH, dmso-d6) δ ppm 9.97 (s, 1H), 7.95 (m, 2H), 7.81 (s, 1H), 7.32 (m, 2H), 5.02 (s, 2H), 3.97 (s, 3H)

¹³C-NMR (125 MHz, dmso-d6) δ ppm 192.3, 159.4, 158.6, 153.1, 142, 132.8, 131.8, 121, 118.5, 54.6

### Preparation R3m: 4-(4-chloro-3-methoxy-phenoxy)-6-methoxy-pyrimidin-5-amine

Using General Procedure 2 starting from Preparation R2m as reagent, Preparation R3m was obtained. HRMS calculated for C₁₂H₁₂ClN₃O₃: 281.0567: found 282.0637 ((M+H)⁺ form).

¹H-NMR (500 MHz, dmso-d6) δ ppm 7.77 (s, 1H), 7.42 (d, 1H), 6.99 (d, 1H), 6.73 (dd, 1H), 4.89 (s, 2H), 3.95 (s, 3H), 3.82 (s, 3H)

¹³C-NMR (125 MHz, dmso-d6) δ ppm 158.1, 155.6, 154.3, 154, 142.8, 130.3, 117.4, 117, 114, 1, 106.9

### Preparation R3n: 4-(4-cyclopropyl-3-methoxy-phenoxy)-6-methoxy-pyrimidin-5-amine

Using General Procedure 2 starting from Preparation R2n as reagent, Preparation R3n was obtained. HRMS calculated for C₁₅H₁₇N₃O₃: 287.127; found 288.13500 ((M+H)⁺ form),

¹H-NMR (5(0 MHz, dmso-d6) δ ppm 7.74 (s, 1H), 6.82 (d, 1H), 6.74 (d, 1H), 6.58 (dd, 1H), 4.8 (s, 2H), 3.95 (s, 3H), 3.77 (s, 3H), 2.05 (m, 1H), 0.86/0.59 (m+n, 2H)

¹³C-NMR (125 MHz, dmso-d6) δ ppm 142.9, 125.3, 113, 104.7, 56.1, 54.4, 9.5.8

### Preparation R4a: 5-amino-4-(4-chlorophenoxy)-1H-pyrimidin-6-one hydrochloride

Using General Procedure 3 starting from Preparation R3a as reagent, Preparation R4a was obtained. HRMS calculated for C₁₀H₈ClN₃O₂: 237.0305; found 238.0379 ((M+H)⁺ form).

¹H-NMR (500 MHz, dmso-d6) δ ppm 12.92 (brs, 1H), 7.8 (s, 1H), 7.44 (m, 2H), 7.14 (m, 2H).
¹³C-NMR (125 MHz, dmso-d6) δ ppm 1594, 153, 152.9, 141.8, 129.8, 128.8, 122.5.

### Preparation R4b: 5-amino-4-(3-chloro-5-methoxy-phenoxy)-1H-pyrimidin-6-one hydrochloride

Using General Procedure 3 starting from Preparation R3b as reagent, Preparation R4b was obtained. HRMS calculated for C₁₁H₁₀ClN₃O₃: 2670411; found 268,0482 ((M+H)⁺ form).

¹H-NMR (400 MHz, dmso-d6) δ ppm 13.18 (brs, 1H), 7.94 (s, 1H), 7.64 (brs, 3H), 6.9 (dd, 1H), 6.84 (dd, 1H), 6.75 (dd, 1H).

¹³C-NMR (100 MHz, dmso-d6) δ ppm 161.3, 159.5, 155.3, 155.3, 144.2, 134.4, 113.5, 111, 106.5.

### Preparation R4c: 5-amino-4-(4-chloro-3-fluoro-phenoxy)-1H-pyrimidin-6-one hydrochloride

Using General Procedure 3 starting from Preparation R3c as reagent. Preparation R4c was obtained. HRMS calculated for C₁₀H₇ClFN₃O₂: 255.0211: found 256.0283 ((M+H)⁺ form).

¹H-NMR (400 MHz, dmso-d6) δ ppm 12.97 (brs, 1H), 7.82 (s, 1H), 7.61 (t, 1H), 7.31 (dd, H), 7.03 (ddd, 1H), 6.74 (brs, 3H).

¹³C-NMR (100 MHz, dmso-d6) δ ppm 159.5, 157.7, 154, 152.1, 141.7, 131.2, 117.9, 115.2, 109.9.

### Preparation R4d: 5-amino-4-(4-fluoro-3-methoxy-phenoxy)-1H-pyrimidin-6-one hydrochloride

Using General Procedure 3 starting from Preparation R3d as reagent, Preparation R4d was obtained. HRMS calculated for C₁₁H₁₀FN₃O₃: 251.0706; found 252.0777 ((M+H)⁺ form).

¹H-NMR (500 MHz, dmso-d6) δ ppm 12.79 (br., 1H), 7.75 (s, 1H), 7.2 (dd, 1H), 6.97 (dd, 1H), 6.64 (ddd, 1H), 5.91 (br., 2H), 3.8 (s, 3H).

¹³C-NMR (125 MHz, dmso-d6) δ ppm 159.4, 152.3, 150.6, 148.9, 148, 140.5, 116.2, 112.3, 107.3, 56.7.

### Preparation R4e: 5-amino-4-3-(trifluoromethyl)phenoxyl-1H-pyrimidin-6-one hydrochloride

Using General Procedure 3 starting from Preparation R3e as reagent, Preparation R4e was obtained. HRMS calculated for C₁₁H₈F₃N₃O₂: 271.0569; found 272.0634 ((M+H)⁺ form).

¹H-NMR (500 MHz, dmso-d6) δ ppm 12.73 (brs, 1H), 7.67 (s, 1H), 7.61 (1, 1H), 7.51 (dm, 1H), 7.42 (m, 1H), 7.39 (dm, 1H).

¹³C-NMR (125 MHz, dmso-d6) δ ppm 159.4, 155.1, 148.9, 138.3, 131.3, 130.6, 124.1, 120.8, 116.7.

### Preparation R4f: 5-amino-4-[3-(trifluoromethoxy)phenoxy]-1H-pyrimidin-6-one

Using General Procedure 3 starting from Preparation R3N as reagent, Preparation R4f was obtained. HRMS calculated for C₁₁H₈F₃N₃O₃: 287.0518: found 288.0592 ((M+H)⁺ form).

¹H-NMR (500 MHz, dmso-d6) δ ppm 12.81 (brs, 1H), 7.73 (s, 1H), 7.51 (t, 1H), 7.17 (dm, 1H), 7.14 (dn, 1H) 7.14 (m, 1H).

¹³C-NMR (025 MHz, dmso-d6) δ ppm 159.5, 155.5, 150.5, 149.2, 139.9, 121.5, 121.3, 119.3, 116.8, 113.3.

### Preparation R4g: 5-amino-4-phenoxy-1H-pyrimidin-6-one

Using General Procedure 3 starting from Preparation R3g as reagent, Preparation R4g was obtained. HRMS calculated for C₁₀H₉N₃O₂: 203.0695: found 204.077 ((M+H)⁺ form).

¹H-NMR (500 MHz, dmso-d6) 3 ppm 12.45 (brs, 1H), 7.5 (s, 1H), 7.35 (m, 2H), 7.11 (m, 1H), 7.02 (m, 2H), 4.6 (s, 2H).

¹³C-NMR (125 MHz, dmso-d6) δ ppm 159.3, 147.3, 135.7, 129.8, 123.8, 121.7, 119.7.

¹⁵N-NMR (50 MHz, dmso-d6) δ ppm 233, 171, 39.

### Preparation R4h: 5-amino-4-(4-fluorophenoxy)-1H-pyrimidin-6-one hydrochloride

Using General Procedure 3 starting from Preparation R3h as reagent, Preparation R4h was obtained. HRMS calculated for C₁₀H₈FN₃O₂: 221.0601: found 222.0669 ((M+H)⁺ form).

¹H-NMR (500 MHz, dmso-d6) δ ppm 7.87 (s, 1H), 7.2 (m, 2H), 7.2 (m, 2H).

¹³C-NMR (125 MHz, dmso-d6) δ ppm 160.7, 159.5, 158.2, 149.8, 143.4, 122.9, 116.6.

### Preparation R4j: 4-[(5-amino-6-oxo-1H-pyrimidin-4-yl)oxy]benzonitrile

Using General Procedure 3 starting from Preparation R3j as reagent. Preparation R4j was obtained. HRMS calculated for C₁₁H₈N₄O₂: 228.0647: found 229.0718 ((M+H)⁺ form).

¹H-NMR (500 MHz, dmso-d6) δ ppm 12.54 (s, 1H), 7.82 (dm, 2H), 7.55 (s, 1H), 7.18 (din, 2H), 4.93 (s, 2H).

¹³C-NMR (125 MHz, dmso-d6) δ ppm 159.5, 159.1, 145.1, 135.6, 134.5, 123.3, 119.7, 119.3, 105.8.

### Preparation R4k: 5-amino-4-(3-pyridyloxy)-1H-pyrimidin-6-one

Using General Procedure 3 starting from Preparation R3k as reagent, Preparation R4k was obtained. HRMS calculated for C₉H₈N₄O₂: 204.0647: found 205.0722 ((M+H)⁺ form).

¹H-NMR (400 MHz, dmso-d6) δ ppm 12.5 (brs, 1H), 8.38 (dd, 1H), 8.34 (dm, 1H), 7.52 (s, 1H), 7.49 (dm, 1H), 7.4 (dd, 1H), 4.73 (s, 2H)

### Preparation R4l: 4-[(5-amino-6-oxo-1H-pyrimidin-4-yl)oxy]benzaldehyde

Using General Procedure 3 starting from Preparation R31 as reagent, Preparation R4l was obtained. HRMS calculated for C₁₁H₉N₃O₃: 231.0644: found 232.0714 ((M+H)⁺ form).

¹H-NMR (400 MHz, dmso-d6) δ ppm 12.55 (brs, 1H), 9.94 (s, 1H), 7.92 (d, 2H), 7.57 (s, 1H), 7.21 (d, 2H), 4.82 (brs, 2H)

¹³C-NMR (100 MHz, dmso-d6) δ ppm 192.1, 159.6, 131.9, 119.2

### Preparation R4m: 5-amino-4-(4-chloro-3-methoxy-phenoxy)-1H-pyrimidin-6-one hydrochloride

Using General Procedure 3 starting from Preparation R3n as reagent, Preparation R4m was obtained. HRMS calculated for C₁₁H₁₀ClN₃O₃: 267.0411; found 268.0481 ((M+H)⁺ form).

¹H-NMR (500 MHz, dmso-d6) δ ppm 12.87 (brs, 1H), 7.78 (s, 1H), 7.4 (d, 1H), 6.86 (d, 1H), 6.68 (dd, 1H), 3.82 (s, 3H)

¹³C-NMR (125 MHz, dmso-d6) δ ppm 141.1, 130.3, 113.2, 106.2, 56.8

### Preparation R4n: 5-amino-4-(4-cyclopropyl-3-methoxy-phenoxy)-1H-pyrimidin-6-one

Using General Procedure 3 starting from Preparation R3n as reagent, Preparation R4n was obtained. HRMS calculated for C₁₄H₁₅N₃O₃: 273.1113: found 274.1183 ((M+H)⁺ form).

¹H-NMR (500 MHz, dmso-d6) δ ppm 12.42 (s, 1H), 7.49 (s, 1H), 6.77 (d, 1H), 6.66 (d, 1H), 6.47 (dd, 1H), 4.54 (s, 2H), 3.76 (s, 3H), 2.01 (m, 1H), 0.84/0.56 (m+m, 4H)

¹³C-NMR (125 MHz, dmso-d6) δ ppm 135.7, 125.3, 111.3, 103.4, 56, 9.5, 7.9

### Preparation R5a: tert-butyl (3R,4R)-4-(1-oxa-6-azaspiro[2.5]octane-6-carbonyl)-3-phenyl-piperidine-1-carboxylate

### Step 1: tert-butyl (3R,4R)-4-[(4-oxopiperidin-1-yl)carbonyl]-3-phenylpiperidine-1-carboxylate

4-piperidone hydrochloride hydrate (0.969 g, 6.3 mmol), EDC.HCl (3.623 g, 18.9 mmol) and (3R,4R)-1-tert-butoxycarbonyl-3-phenyl-piperidine-4-carboxylic acid (1.928 g, 6.3 mmol) were dissolved in pyridine (10 mL) and stirred at r.t. for 16 hours. The reaction mixture was evaporated to Celite and purified by flash chromatography (DCM:MeOH, gradient) to give the product of the title. HRMS calculated for C₂₂H₃₀N₂O₄: 386.2206; found 409.2093|(M+Na)⁺ form|.

¹H-NMR (500 MHz, DMSO-d6): δ 1.42 (s, 9H), 4.14-1.50 (m, 16H), 7.32-7.15 (m, 5H).

### Step 2: Preparation R5a

tert-butyl (3R,4R)-4-[(4-oxopiperidin-1-yl)carbonyl]-3-phenylpiperidine-1-carboxylate (5 g, 155 mmol, 1.0 eq.) and trimethylsulfoxonium-iodide (85.41 g, 388 mmol, 2.5 eq.) was charged into a round bottom flask and dissolved/suspended in MeCN (150 ml) and MTBE (150 ml). NaOH (15.5 g, 388 mmol, 2.5 eq.) was dissolved in water (21.6 ml) (˜40% solution). The aq. NaOH solution was added to the mixture and stirred at 60° C. for 2 hours. After the reaction completed, the mixture was cooled to r.t., filtered through a Celite bed, the filter cake was washed with MTBE (2×60 ml). Water (150 ml) was added to the organic layer and after extraction the layers were separated. The aq. layer was extracted with further MTBE (2×60 ml). The combined organic layers were dried over MgSO₄ and after filtration evaporated to give Preparation R5a as beige solid foam. HRMS calculated for C₂₃H₃₂N₂O₄: 400.2362; found 423.2247 [(M+Na)⁺ form].

¹H-NMR (500 MHz, DMSO-d6): δ=1.41 (s, 9H), 1.79-0.86 (m, 6H), 2.61-2.51 (m, 2H), 4.16-2.73 (m, 10H), 7.33-7.18 (m, 5H),

### Preparation R5b: tert-butyl (3R,4R)-4-(4,4-difluoro-1-oxa-6-azaspiro[2.5]octane-6-carbonyl)-3-phenyl-piperidine-1-carboxylate

### Step 1: tert-butyl (3R,4R)-4-(3,3-difluoro-4,4-dihydroxy-piperidine-1-carbonyl)-3-phenyl-piperidine-1-carboxylate

3,3-Difluoropiperidin-4-one hydrochloride hydrate (3.61 g, 19.041 mmol), EDC.HCl (10.951 g, 57.123 mmol) and (3R,4R)-1-tert-butoxycarbonyl-3-phenyl-piperidine-4-carboxylic acid (6.397 g, 20.945 mmol) were dissolved in pyridine (80 mL) and stirred at r.t. for 17 hours. The reaction mixture was evaporated under reduced pressure. It was dissolved in DMF and then it was purified by preparative LC (on C-18 Luna 10 pun column, 5 mM aqueous NH₄HCO₃-MeCN, gradient). Solvent was evaporated under reduced pressure to give the product of the title. HRMS calculated for C₂₂H₃₀F₂N₂O₅: 440.2123: found 463.2008 [(M+Na)⁺ form].

### Step 2: Preparation R5h

tert-butyl (3R,4R)-4-(3,3-difluoro-4,4-dihydroxy-piperidine-1-carbonyl)-3-phenyl-piperidine-1-carboxylate (1.048 g, 2.379 mmol, 1.0 eq.) and trimethylsulfoxonium-iodide (1.309 g, 5.948 mmol, 2.5 eq.) was charged into a round bottom flask and dissolved/suspended in MeCN (30 ml) and MTBE (30 ml), NaOH (238 mg 5,948 mmol, 2.5 eq.) was dissolved in water (7 ml). The aq. NaOH solution was added to the mixture and stirred at 60° C. for 2 hours. After the reaction completed, the mixture was cooled to r.t, 15 g MgSO₄ and 30 ml MTBE were added the mixture was filtered after 10 minutes, the inorganic solid was washed with 2×30 ml MTBE, the mother liquor was evaporated under reduced pressure. Then 10 g MgSO₄ and DCM were added, then it was filtered and washed with 20 mL DCM. The organic solvent was evaporated under reduced pressure to give Preparation R5b. HRMS calculated for C₂₃H₁₀F₂N₂O₄: 436.2174; found 459.20595 [(M+Na)⁺ form].

### Preparation R6a: 3-fluoro-5-iodo-thiophene-2-carboxylic acid

### Step 1: 3-fluoro}-5-iodo-thiophene-2-carboxylate and methyl 3-fluoro-4,5-diiodo-thiophene-2-carbaoxylate

To the solution of methyl 3-fluorothiophene-2-carboxylate (2.42 g, 15.1 mmol) in THF=(10 ml), TMP.MgCl.LiCl (1N in THF/toluene, 25 ml, 25 mmol) was added dropwise at −45° C. in 5 minutes. After 30 minutes of stirring, iodine (4.04 mg, 15.9 mmol) was added in THF (10 ml) at −45° C. to the mixture. After warming up (1 hour), sat. NH₄Cl solution (50 ml, aq.) was added to the mixture while stirring. The mixture was extracted with DEE (5×10 ml). The combined organic layer was evaporated. The residue was purified by preparative LC (on C-18 Gemini-NX 5 μm column, 5 mM aqueous NH₄HCO₃-MeCN, gradient) to give 3-fluoro-5-iodo-thiophene-2-carboxylate and methyl 3-fluoro-4,5-diiodo-thiophene-2-carboxylate, separately.

### Methyl 3-fluoro-5-iodo-thiophene-2-carboxylate

GC-MS calculated for C₆H₄FIO₂S: 285.8961; found 285.9 [(M, Et) form].

¹H-NMR (500 MHz, DMSO-d6): δ ppm 7.52 (s, 1H), 3.78 (s, 3H).

¹³C-NMR (125 MHz DMSO-d6): δ ppm 159.4, 158.8, 128.5, 117.2, 86.4, 52.8.

### Methyl 3-fluoro-4,5-diiodo-thiophene-2-carboxylate

GC-MS calculated for C₆H₄FIO₂S: 411.7927: found 411.9 [(M, EI) form].

¹H-NMR (500 MHz, DMSO-d6): δ ppm 3.8 (s, 3H).

¹³C-NMR (125 MHz, DMSO-d6): δ ppm 159, 158.2, 117.4, 96.1, 90.6, 53.1.

### Step 2: Preparation R6a

Methyl 3-fluoro-5-iodo-thiophene-2-carboxylate (1.188 g, 4.135 mmol), lithium-hydroxide monohydrate (867 mg, 20.7 mmol) were stirred in methanol (10 ml) and water (10 ml) at 70° C. for 1 hour. The mixture was partially evaporated and the aqueous residue was acidified with 1N HCl (25 ml, aq.). The resulted precipitate was filtered off, washed with water and dried to give Preparation R6a. GC-MS calculated for C₅H₂FIO₂S: 271.8804; found 271.9 [(M, EI) form].

¹H-NMR (500 MHz, DMSO-d6): n ppm 13.48 (brs, 1H), 7.74 (s, 1H).

¹³C-NMR (125 MHz, DMSO-d6): δ ppm 160.4, 158.3, 128.5, 119, 85, 1.

### Preparation R6b: 4-bromo-5-chloro-3-fluoro-thiophene-2-carboxylic acid

### Step 1: methyl 5-bromo-3-fluoro-thiophene-2-carboxylate

To the solution of methyl 3-fluorothiophene-2-carboxylate (320 mg, 2 mmol) in THF (2 ml), TMP.MgCl.LiCl (1N in THF/toluene, 4 ml, 4 mmol) was added dropwise at −45° C. in 5 minutes. After 25 minutes of stirring 1,2-dibromo-1,1,2,2-tetrachloro-ethane (716 mg, 2.2 mmol) was added in THF (2 ml) at −45° C. to the mixture. After warming up (30 minutes), sat. NH₄Cl solution (5 ml, aq.) was added to the mixture while stirring. The mixture was extracted with ethyl acetate (3×10 ml). The combined organic layer was evaporated. The residue was purified by preparative LC (on C-18 Gemini-NX 5 μm column, 5 mM aqueous NH₄HCO₃-MeCN, gradient) to give the product of the title. GC-MS calculated for C₆H₄BrFO₂S: 237.9099; found 237.8 [(M, EI) form].

¹H-NMR (500 MHz, DMSO-d6): δ ppm 7.52 (s, 1H), 3.80 (s, 3H).

¹³C-NMR (125 MHz, DMSO-d6): δ ppm 159.5, 158.2, 123.2, 119.6, 113.8, 53.0.

### Step 2: methyl 4-bromo-5-chloro-3-fluoro-thiophene-2-carboxylate

To the solution of methyl 5-bromo-3-fluoro-thiophene-2-carboxylate (210 mg, 0.878 mmol) in THE (2 ml) at TMP.MgCl.LiCl (1N in THF/toluene, 2 ml, 2 mmol) was added dropwise at −45° C. in 5 minutes. After 20 minutes of stirring, 1,1,1,2,2,2-hexachloroethane (236 mg, 1 mmol) was added in THF (2 ml) at −45° C. to the mixture. After warming up (30 minutes), sat. NH₄Cl solution (5 ml, aq.) was added to the mixture while stirring. The mixture was extracted with ethyl acetate (3×10 ml). The combined organic layer was evaporated. The residue was purified by preparative LC (on C-18 Gemini-NX 5 μm column, 5 mM aqueous NH₄HCO₃-MeCN, gradient) to give the product of the title. ¹H-NMR (500 MHz, dmso-d6) δ ppm 3.84 (s, 3H)

¹³C-NMR (125 MHz, dmso-d6) δ ppm 159, 154.4, 132.2, 111.3, 104.2, 53.4

¹⁹F-NMR (376.5 MHz, dmso-d6) δ ppm −107.25

### Step 3: Preparation R6b

Methyl 4-bromo-5-chloro-3-fluoro-thiophene-2-carboxylate (116 mg, 0.424 mmol), lithium-hydroxide monohydrate (53.3 mg, 1.27 mmol, 3.0 eq.) were stirred in methanol (1.5 ml) and water (1.5 ml) at 50° C. for 20 hours. The mixture was partially evaporated and the aqueous residue was acidified with 1N HCl (aq.). The resulted precipitate was filtered off, washed with water and dried to give Preparation R6b.

¹H-NMR (500 MHz, MSM-d6): δ (ppm) 14.07 (brs, 1H).

¹³C-NMR (125 MHz, MSM-d6): d (ppm) 160.0, 153.8, 131.2, 113.4, 103.9.

¹⁹F-NMR (376.48 MHz, MSM-d6): S (ppm) −108.9

### Preparation R7a: ethyl (3R,4R)-3-phenylpiperidine-4-carboxylate

### Step 1: ethyl (3R,4R)-3-phenylpiperidine-4-carboxylate hydrochloride

In a three-necked 100 ml flask under N₂ atmosphere, (3R,4R)-1-(tert-butoxycarbonyl)-3-phenylpiperidine-4-carboxylic acid (953 mg; 3.12 mmol; 1.0 eq.) was suspended in abs. EtOH (18.2 ml; 312 mmol; 100 eq.). After cooling to 0° C. with ice bath, thionyl-chloride was added via syringe at such rate that the temperature is kept under 50° C. Then, the suspension was stirred for 60 hours at 50° C. The solution was cooled to r.t., when white crystals formed in the flask. The precipitate was collected by filtration; the filtrate was stored at −18° C. for 2 hours when most of the product precipitated and it was filtered again. The combined white crystals were washed with ice-cold EtOH (5 ml) and DEE (2×10 ml), then dried in vacuum to give the product of the title.

### Step 0.2: Preparation R7a

In a one-necked 50 mi flask, hydrochloride salt of (3R,4R)-ethyl 3-phenylpiperidine-4-carboxylate (270 mg; 1.0 mmol; 1.0 eq.) was dissolved in H₂O (5 ml), DCM (10 ml) was added to the solution. Then, NaHCO₃ dissolved in water (0.6M; 252 mg; 3.0 mmol; 3.0 eq.) was added to the biphasic system. The mixture was stirred vigorously for 40 minutes. Organic layer was separated; inorganic phase was extracted with DCM (3×10 ml). The combined organic phase was washed with brine (5 ml), dried over Na₂SO₄, filtered and evaporated to dryness to give Preparation R7a. HRMS calculated for C₁₄H₁₉NO₂: 233.1416: found 234.1487 ((M+H)⁺ form).

¹H-NMR (500 MHz, dmso-d6) δ ppm 7.29-7.14 (m, 5H), 3.8 (q, 2H), 2.98/2.57 (dm+m, 2H), 2.87/2.53 (dm+m, 2H), 2.74 (m, 1H), 2.74 (m, 1H), 2.31 (vbrs, 1H), 1.8/1.54 (dm+m, 2H), 0.87 (t, 3H)

¹³C-NMR (125 MHz, dmso-d6) h ppm 59.8, 53.3, 48.1, 46.6, 45.4, 30.4, 14.3

### Preparation R7b: (3R,4R)-ethyl 1-(3-(1H-benzo[d][1,2,3]triazol-1-yl)oxetan-3-yl)-3-phenylpiperidine-4-carboxylate

In a one-necked 50 ml flask, to a stirred solution of oxetan-3-one (288 mg: 4.0 mmol; 2.0 eq.) and Preparation R7a (467 mg; 2.0 mmol; 1.0 eq.) in amylene stabilized DCM (0.2M; 10 ml), was added 1H-benzo[d]1,2,3]triazole (262 mg; 2.2 mmol; 1.1 eq.) at r.t. The mixture was stirred at r.t. for 90 minutes and evaporated to dryness at 50° C. The crude product was used in the following step without any purification.

### Preparation R8a: (3R,4R)-3-phenyl-1-(3-phenyloxetan-3-yl)piperidine-4-carboxylic acid

### Step 1: ethyl (3R,4R)-3-phenyl-1-(3-phenyloxetan-3-yl)piperidine-4-carboxylate

Phenylmagnesium bromide (0.3M; 6 mL; 1.8 mmol; 2.0 eq.) was added via syringe into a flame-dried, 3-necked 1100 mL flask equipped with thermometer, nitrogen inlet and septum. ZnCl₂ (2.0M Me-THF solution; 0.540 mL; 1.08 mmol; 1.2 eq.) was added dropwise to the Grignard solution keeping the temperature between 25-30° C. The formed organozinc reagent was stirred at r.t. for 15 minutes, followed by the addition of Preparation R7b (0.2M abs. THF; 4.5 ml; 0.9 mmol; 1.0 eq.) in a rate that the temperature was kept between 25-30° C. with water bath. The solution was stirred for 30 minutes at rt, then quenched with water (3 mL). DEE (20 mL) and saturated Na₂CO₃ (3 mL) was added to the suspension and stirred for further 5 minutes. The white suspension was filtered through a short pad of Celite which was washed with 3×15 mL DEE. The filtrate was washed with saturated Na₂CO₃ (5 mL), dried on Na₂SO₄ and concentrated on a rotary evaporator under reduced pressure to dryness. The crude product was purified by flash chromatography (hexanes/ethyl acetate 3/1). Fractions were evaporated to give ethyl (3R,4R)-3-phenyl-1-(3-phenyloxetan-3-yl)piperidine-4-carboxylate. HRMS calculated for C₂₃H₂₇NO₃: 365.1991; found 366.2062 ((M+H)⁺ form).

¹H-NMR (500 MHz, dmso-d6) δ ppm 7.42-7.09 (m, 10H), 4.79-4.69 (m, 4H), 3.8 (m, 2H), 3.44 (td, 1H), 3.01 (td, 1H), 2.9311.62 (d+td, 2H), 2.81/L54 (dm+t, 2H), 1.89/1.75 (dm+qd, 2H), 0.85 (t, 3H)

¹³C-NMR (125 MHz, dmso-d6) δ ppm 174.1, 80.1/79.9, 66.6, 59.9, 53.1, 47.2, 45.8, 45.6, 29.4, 14.3

### Step 2: Preparation R8a

Ethyl (3R,4R)-3-phenyl-1-(3-phenyloxetan-3-yl)piperidine-4-carboxylate (375 mg, 1.026 mmol), lithium hydroxide monohydrate (172 mg, 4,104 mmol, 4.0 eq.) were stirred in ethanol (5 ml) and water (5 ml) at r.t. for 28 hours. The mixture was partially evaporated and the aqueous residue was acidified with 1N aqueous HCl solution. The resulted precipitate was filtered off, washed with water and dried to give Preparation R8a. HRMS calculated for C₂₁H₂₃NO₃: 337.1678; found 338.1743 ((M+H)⁺ form).

¹H-NMR (500 MHz, dmso-d6) δ ppm 11.89 (s, 1H), 3 (m, 1H), 7.41-7.08 (m, 10H), 4.81-4.68 (m, 4H), 2.93/1.59 (m+m, 2H), 2.79/1,48 (m+m, 2H), 2.36 (m, 1H), 1.93/1.73 (m+m, 2H)

¹³C-NMR (125 MHz, dmso-d6) δ ppm 66.6, 53.4, 47, 45.8, 45.4, 29.7

### Preparation R8b: (3R,4R)-1-[3-(2-fluorophenyl)oxetan-3-yl]-3-phenyl-piperidine-4-carboxylic acid

### Step 1: Preparation of (2-fluorophenyl)magnesium bromide

In a three-necked 100 ml flask under N_{z} atmosphere, 1-bromo-2-fluorobenzene (0.984 ml; 9.0 mmol; 6.0 eq.) was dissolved in 18 ml abs. THF. The solution was cooled to −10° C. Then, isopropylmagnesium chloride solution (0.5M in abs. THF; 18 ml; 9.0 mmol; 6.0 eq.) was added via syringe, while keeping the temperature under −5° C. The Grignard reagent was kept between −10° C. and −5° C. at this temperature and used up immediately to the next step.

### Step 2: Ethyl (3R,4R)-1-(3-(2-fluorophenyl)oxetan-3-yl-3-phenylpiperidin-4-carboxylate

In a three-necked 100 ml flask under N₂ atmosphere, a (2-fluorophenyl)magnesium bromide solution (0.25M in abs. THF; 36 ml; 9.0 mmol; 6.0 eq.) was prepared according to Step 1 above. To the freshly prepared Grignard reagent, ZnCl₂ solution (2.0M in abs. Me-THF; 2.5 ml: 4.9 mmol; 3.3 eq.) was added via syringe at such rate that the temperature is kept between −10° C. and −5° C. After stirring 5 minutes the clear solution, Preparation R7b (0.2M abs. THF; 7.5 ml: 1.5 mmol; 1.0 eq.) was added dropwise to the organozinc reagent, keeping the temperature still between −10° C. and −5° C. The reaction mixture was allowed to warm to r.t. in 20 minutes then quenched with 3 ml cc. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml cc. Na₂CO₃ solution. Organic phase was dried over anhydrous Na₂SO₄ then evaporated to dryness. Purification by flash chromatography (85/15 hexanes/ethyl acetate) afforded ethyl (3R,4R)-1-(3-(2-fluorophenyl)oxetan-3-yl)-3-phenylpiperidine-4-carboxylate as a colourless oil,

### Step 3: Preparation R8b

Ethyl (3R,4R)-1-(3-(2-fluorophenyl)oxetan-3-yl)-3-phenylpiperidine-4-carboxylate (300 mg, 0.782 mmol), lithium hydroxide monohydrate (65.6 mg, 1.565 mmol, 2.0 eq.) were stirred in ethanol (3 ml) and water (3 mi) at r.t, for 56 hours. The mixture was partially evaporated and the aqueous residue was acidified with 1 N HCl (aq.). The resulted precipitate was filtered off, washed with water and dried to give Preparation R8b. HRMS calculated for C₂₁H₂₂FNO₃: 355.1584; found 356.1653 ((M+H)⁺ form).

¹H-NMR (500 MHz, dmso-d6) δ ppm 11.93 (brs, 1H), 7.37 (dd, 1H), 7.25 (1, 2H), 7.21 (t, 1H), 7.2 (t, 1H), 7.19 (d, 2H), 7.18 (t, 1H), 7.12 (t, 1H), 4.81/4.76/4.74 (d+d+s, 4H), 3.03/1.64 (d+t, 2H), 3.02 (t, 1H), 2.89/1.53 (d+t, 2H), 2.42 (t, 1H), 1.95/1.75 (ddd+ddd, 2H),

¹³C-NMR (125 MHz, dmso-d6) δ ppm 175.8, 160.6, 142.7, 130.6, 130.2, 128.7, 128.2, 127, 124.4, 123.7, 116.2, 79.2/79, 64.8, 53.3, 47, 45.8, 45.3, 29.7

### Preparation R8c: (3R,4R)-1-[3-(4-fluorophenyl)oxetan-3-yl]-3-phenyl-piperidine-4-carboxylic acid

### Step 1: ethyl (3R,4R)-1-[3-(4-fluorophenyl)oxetan-3-yl]-3-phenyl-piperidine-4-carboxylate

To a three-necked 100 ml flask under N₂ atmosphere. (4-fluorophenyl)magnesium bromide solution (0.5M in abs. THF; 24 ml; 12.0 mmol; 6.0 eq.) was added via syringe. The Grignard solution was cooled to −10° C. with ice-NaCl cooling bath and ZnCl₂ solution (2.0M in abs. Me-THF: 3.3 ml: 6.6 mmol; 3.3 eq.) was added via syringe at such rate that the temperature was kept between −10° C. and −5° C. After stirring 5 minutes the clear solution, Preparation R7b (0.2M abs. THF; 10 ml: 2.0 mmol; 1.0 eq.) was added dropwise to the organozinc reagent, keeping the temperature still between −10° C. and −5° C. The reaction mixture is allowed to warm to r.t. in 20 minutes then quenched with 5 mil cc. Na₂CO₃ solution while keeping the temperature under 30° C. To the resulting suspension. 40 ml DCM was added, then stirred vigorously for 5 minutes. The slurry was filtered through a short pad of Celite, diluted with 60 mi DCM then washed with 10 ml cc. Na₂CO₃ solution. Organic phase was dried over anhydrous Na₂SO₄ then evaporated to dryness. Purification by flash chromatography (4/1 hexanes ethyl acetate) afforded ethyl (3R,4R)-1-[3-(4-fluorophenyl)oxetan-3-yl]-3-phenyl-piperidine-4-carboxylate. HRMS calculated for C₂₃H₂₆FNO₃: 383.1897; found 384.196 ((M+H)⁺ form).

¹H-NMR (500 MHz, dmso-d6) δ ppm 7.24 (t, 2H), 7.21 (dd, 2H), 7.2 (t, 2H), 7.19 (t, 1H), 7.16 (d, 2H), 4.74/4.69 (d+d, 2H), 4.73 (s, 2H), 3.8 (q, 2H), 3 (t, 11-), 2.92/1.59 (dd+t, 2H), 2.8/1.53 (dd+d, 2H), 2.46 (t, 1H) 1.89/1.74 (ddd+ddd, 2H), 0.85 (t, 3H).

¹³C-NMR (125 MHz, dmso-d6) δ ppm 174.1, 161.6, 142.1, 133.5, 129.9, 128.7, 128.2, 127.1, 114.9, 80.1, 79.9, 66.2, 59.9, 53, 47.2, 45.8, 45.5, 29.4, 14.3

### Step 2: Preparation R8c

Ethyl (3R,4R)-1-[3-(4-fluorophenyl)oxetan-3-yl]-3-phenyl-piperidine-4-carboxylate (130 mg, 0.339 mmol), lithium hydroxide monohydrate (28.4 mg, 0.678 mmol, 2.0 eq.) were stirred in ethanol (3 ml) and water (3 ml) at 80° C. for 41 hours. The mixture was partially evaporated and the aqueous residue was acidified with 1N HCl (aq.). The resulted precipitate was filtered off, washed with water and dried to give Preparation R8c. HRMS calculated for C₂₁H₂₂FNO₃: 355.1584; found 356.1659 ((M+H)⁺ form).

¹H-NMR (400 MHz, dmso-d6) δ ppm 7.40-7.10 (br., 9H), 3.01 (br., 1H), 4.90-4.60 (br., 4H), 2.9211.58 (br.+br., 2H), 2.79/1.48 (br.+br., 2H), 2.39 (br., 1H), 1.94/1.73 (br.+br., 2H)

¹³C-NMR (100 MHz, dmso-d6) δ ppm 80, 53.3, 47.1, 45.8, 45.4, 29.7

### Preparation R8d: (3R,4R)-1-(3-methyloxetan-3-yl)-3-phenyl-piperidine-4-carboxylic acid

### Step 1: ethyl (3R,4R)-1-(3-methyloxetan-3-yl)-3-phenyl-piperidine-4-carboxylate

To a three-necked 100 ml flask under N: atmosphere, a methylmagnesium chloride solution (0.5M in THF; 24 ml: 12.0 mmol: 6.0 eq.) was added via syringe. The Grignard solution was cooled to −10° C. with ice-NaCl cooling bath and ZnCl₂ solution (2.0M in abs. Me-THF; 3.3 ml; 6.6 mmol: 3.3 eq.) was added via syringe at such rate that the temperature is kept between −10° C. and 5° C. After stirring 5 minutes, Preparation R7b (0.2M abs. THF; 10 ml; 2.0 mmol; 1.0 eq.) was added dropwise to the organozinc reagent, keeping the temperature still between −10° C. and −5° C. The reaction mixture is allowed to warm to r.t. in 20 minutes then quenched with 5 ml cc. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml cc. Na₂CO₃ solution. Organic phase was dried over anhydrous Na₂SO₄ then evaporated to dryness. Purification by flash chromatography (3/1 hexanes/ethyl acetate) afforded ethyl (3R,4R)-1-(3-methyloxetan-3-yl)-3-phenyl-piperidine-4-carboxylate. HRMS calculated for C₁₈H₂₅NO₃: 303.1834; found 304.1909 ((M+H)⁺ form)

¹H-NMR (500 MHz, dmso-d6) δ ppm 7.25 (m, 2H), 7.25 (m, 2H), 7.19 (t, 1H), 4.4/4.06 (d+d, 2H), 4.4/4.11 (d+d, 2H), 3.82 (q, 2H), 2.93 (t, 1H), 2.63 (t, 1H), 2.58/2.18 (dd+td, 2H), 2.46/2.14 (dd+td, 2H), 1.92/1.69 (ddd+ddd, 2H), 1.25 (s, 3H), 0.89 (t, 3H).

¹³C-NMR (125 MHz, dmso-d6) δ ppm 174.2, 142.2, 128, 128.2, 127.1, 81.2, 81.1, 60.1, 59.9, 52.4, 47.6, 45.7, 44.7, 29.6, 14.8, 14.3

### Step 2: Preparation R8d

Ethyl (3R,4R)-1-(3-methyloxetan-3-yl)-3-phenyl-piperidine-4-carboxylate (69 mg, 0,227 mmol), lithium hydroxide monohydrate (19.0 mg, 0.454 mmol, 2.0 eq.) were stirred in ethanol (2 ml) and water (2 ml) at 80° C. for 41 hours. The mixture was partially evaporated and the aqueous residue was acidified with 1N HCl (aq.) and it was extracted with 2×5 mi DCM. The combined organic layers were dried over MgSO₄, Filtered and the filtrate was concentrated under reduced pressure to give Preparation R8c. HRMS calculated for C₁₆H₂₁NO₃: 275.1521; found 276.1592 ((M+H)⁺ form).

¹H-NMR (400/500 MHz, dmso-d6) δ ppm 12.52-11.62 (br., 1H), 7.50-7.0) (br., 5H), 5.20-3.95 (br., 4H), 3.62-2.01 (br., 6H), 2.32-1.57 (br., 2H), 1.64/1.23 (br.+br., 3H)

### Preparation R8e: (3R,4R)-1-(3-isopropyloxetane-3-yl)-3-phenyl-piperidine-4-carboxylic acid

### Step 1: ethyl (3R,4?R)-1-(3-isopropyloxetane-3-yl)-3-phenyl-piperidine-4-carboxylate

To a three-necked 100 ml flask under N2 atmosphere, an isopropylmagnesium chloride solution (0.5M in abs. THF; 31 ml; 15.6 mmol; 6.0 eq.) was added via syringe. The Grignard solution was cooled to −10° C. with ice-NaCl cooling bath, and ZnCl₂ solution (2.0M in abs. Me-THF; 4.3 ml; 6.6 mmol: 3.3 eq.) was added via syringe at such rate that the temperature is kept between −10° C. and −5° C. After stirring 5 minutes the clear solution, Preparation R7b (0.2M abs. THF; 13 ml; 2.6 mmol; 1.0 eq.) was added dropwise to the organozinc reagent, keeping the temperature still between −10° C. and −5° C. The reaction mixture is allowed to warm to r.t. in 20 minutes then quenched with 5 mi cc. Na₂CO₃ solution while keeping the temperature under 30° C. To the resulting suspension 40 ml DCM was added, then stirred vigorously for 5 minutes. The slurry was filtered through a short pad of Celite, diluted with 60 ml DCM then washed with 10 ml cc. Na₂CO₃ solution. Organic phase was dried over anhydrous Na₂SO₄ then evaporated to dryness. Purification by flash chromatography (83/17 hexanes/ethyl acetate) afforded ethyl (3R,4R)-1-(3-isopropyloxetan-3-yl)-3-phenyl-piperidine-4-carboxylate as a colourless oil. HRMS calculated for C₂₀H₂₉NO₃: 331.2148: found 332,22154 ((M+H)⁺ form),

¹H-NMR (500 MHz, dmso-d6) δ ppm 7.25 (m, 2H), 7.25 (m, 2H), 7.19 (t, 1H), 4.52/4.23 (d+d, 2H), 4.47/4.23 (d+d, 2H), 3.82 (q, 2H), 2.89/2.5 (d+t, 2H), 2.86 (t, 1H), 2.7512.49 (dt, 2H), 2.69 (t, 1H), 2.08 (m, 1H), 1.91/1.63 (d+dd, 2H), 0.97 (d, 6H), 0.88 (t, 3H),

¹³C-NMR (125 MHz, dmso-d6) δ ppm 174.3, 142.3, 128.6, 128.2, 127.1, 74.7, 74.5, 66.2, 59.9, 53.9, 47.8, 46.6, 46.3, 31.3, 30.3, 18.4, 14.3

### Step 2: Preparation R8e

Ethyl (3R,4R)-1-(3-isopropyloxetan-3-yl)-3-phenyl-piperidine-4-carboxylate (280 mg, 0.844 mmol), lithium hydroxide monohydrate (106 mg, 2.534 mmol, 3.0 eq.) were stirred in ethanol (3 ml) and water (3 ml) at r.t. for 44 hours. The mixture was partially evaporated and the aqueous residue was acidified with 1N HCl (aq.) and it was evaporated to Celite. Then it was purified via flash chromatography using DCM and MeOH as eluents. Solvents were evaporated under reduced pressure to give Preparation R8e. FIRMS calculated for C₁₈H₂₅NO₃: 303.1834; found 304.1902 ((M+H)⁺ form).

¹H-NMR (500) MHz, dmso-d6) δ ppm (m, 5H), 4.52/4.47/4.24 (br/br.+br, 4H), 2.91/2.5 (br.+br., 2H), 2.87 (br., 1H), 2.75/2.45 (br.+br., 2H), 2.63 (br., 1H), 2.08 (br., 1H), 1.95/1.63 (d+br., 2H), 0.96 (d, 6H)

¹³C-NMR (125 MHz, dmso-d6) δ ppm 74.5, 54.1, 47.4, 46.4, 46, 31, 30.4, 18.4

### Preparation R8f: (3R,4R)-1-(3-(2-methoxyphenyl)oxetan-3-yl)-3-phenyl-piperidine-4-carboxylic acid

### Step 1: (3R,4R)-ethyl 3-phenyl-1-(3-o-tolyl)oxetan-3-yl)piperidine-4-carboxylate

To a three-necked 100 ml flask under N2 atmosphere, ortho-methyl-phenyl-MgBr solution (0.41M in abs. THF; 21 nil: 8.6 mmol; 6.0 eq.) was added via syringe. The Grignard-solution was cooled to −10° C. with ice-NaCl cooling bath, and ZnCl₂-solution (2.0M in abs. 2-methyltetrahydrofuran; 2.3 ml; 4.6 mmol: 3.2 eq.) was added via syringe at such rate that the temperature is kept between −10° C. and −5° C. After stirring 5 minutes. Preparation R7b (0.2M abs. THF; 7.1 ml; 1.43 mmol; 1.0 eq.) was added dropwise to the organozinc reagent, keeping the temperature still between −10° C. and −5° C. The reaction mixture is allowed to warm to r.t. in 20 minutes then quenched with 5 ml sat. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂CO₃ solution. Organic phase was dried over anhydrous Na₂SO₄ then evaporated to dryness. Purification by flash chromatography (4:1 hexanes/EtOAc) afforded (3R,4R)-ethyl 1-(3-(2-methoxyphenyl)oxetan-3-yl)-3-phenylpiperidine-4-carboxylate. HRMS calculated for C₂₄H₂₉NO₃: 379.2148: found 380.2222 ((M+H)⁺ form).

¹H-NMR (500 MHz, dmso-d6) δ ppm 7.29-6.93 (m, 9H), 4.95-4.81 (m, 4H), 3.82/3.79 (m+m, 2H), 3.08/2.22 (m+m, 2H), 2.97/2.23 (m+m. 2H), 2.86 (m, 1H), 2.61 (m, 1H), 2.16 (s, 3H), 1.93/1.64 (m+m, 2H), 0.86 (t, 3H).

¹³C-NMR (125 MHz, dmso-d6) δ ppm 59.9, 53.2, 47.8, 46.1, 45.8, 29.8, 20, 14.3

### Step 2: Preparation R8f

(3R,4R)-ethyl 3-phenyl-1-(3-(o-tolyl)oxetan-3-yl)piperidine-4-carboxylate (369 mg, 0.972 mmol), lithium hydroxide monohydrate (122 mg, 2.917 mmol, 3.0 eq.) were stirred in ethanol (4 ml) and water (4 ml) at 50° C. for 80 hours. The mixture was partially evaporated and the aqueous residue was acidified with 1N HCl (aq.). The resulted precipitate was filtered off, washed with water and dried to give Preparation R8f. HRMS calculated for C₂₂H₂₅NO₃: 351.1834; found 352.1907 ((M+H)⁺ form).

¹H-NMR (500 MHz, dmso-d6) δ ppm 12.3 (brs, 1H), 7.25 (t, 2H), 7.2 (d, 2H), 7.18 (m, 1H), 7.17 (m, 1H), 7.16 (d, 1H), 7.12 (m, 1H), 6.96 (d, 1H), 4.92/4.9/4.85 (d+d+d, 4H), 3.07/2.2 (d+t, 2H), 2.96/2.17 (d+t, 2H), 2.86 (td, 1H), 2.53 (td, 1H), 2.16 (s, 3H), 1.96/1.61 (ddd+ddd, 2).

¹³C-NMR (125 MHz, dmso-d6) δ ppm 175.9, 142.7, 139.1, 136.6, 131.8, 128.8, 128.7, 128.2, 127.7, 127, 125.5, 77.1/77, 68.8, 53.4, 47.6, 46, 45.7, 30.1, 20

### Preparation R8Z: (3R,4R)-1-[3-(2-methoxyphenyl)oxetan-3-yl]-3-phenyl-piperidine-4-carboxylic acid

### Step 1: (3R,4R)-ethyl 1-(3-(2-methoxyphenyl]oxetan-3-yl)-3-phenylpiperidine-4-carboxylate

To a three-necked 100 ml flask under N₂ atmosphere, ortho-methoxy-phenyl-MgBr solution (0.35M in abs, THF; 17 ml: 6.0 mmol: 6.0 eq.) was added via syringe. The Grignard solution was cooled to −10° C. with ice-NaCl cooling bath, and ZnCl₂ solution (200 M in abs, 2-methyltetrahydrofuran; 1.6 ml; 3.2 mmol: 3.2 eq.) was added via syringe at such rate that the temperature is kept between −10° C. and −5° C. After stirring 5 minutes Preparation R7b (0.2M abs. THF; 5 ml; 1.00 mmol: 1.0 eq.) was added dropwise to the organozinc reagent, keeping the temperature still between −10° C. and −5° C. The reaction mixture is allowed to warm to r.t. in 20 minutes then quenched with 5 ml sat. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂CO₃ solution. Organic phase was dried over anhydrous Na₂SO₄ then evaporated to dryness. Purification by flash chromatography (4:1 hexanes/EtOAc) afforded (3R,4R)-ethyl 1-(3-(2-methoxyphenyl)oxetan-3-yl)-3-phenylpiperidine-4-carboxylate. HRMS calculated for C₂₄H₂₉NO₄: 395.2097; found 396.2171 ((M+H)⁺ form).

¹H-NMR (500 MHz, dmso-d6) δ ppm 7.27 (m, 1H), 7.27-7.12 (7, 5H), 7.02 (dm, 1H), 6.92 (m, 1H), 6.88 (dm, 1H), 4.82-4.6 (m, 4H), 3.82/3.78 (m+m, 2H), 3.63 (s, 3H), 3. I/1.65 (m+m, 2H), 2.99 (m, 1H), 2.96/1.62 (m+m, 2H), 2.45 (n, 1H), 1.89/1.75 (m+m, 2H), 0.86 (t, 3H).

¹³C-NMR (125 MHz, dmso-d6) δ ppm 129.7, 129.3, 120.3, 112.3, 59.8, 55.7, 53.3, 47.5, 45.9, 45.9, 29.7, 14.3

### Step 2: Preparation R8g

(3R,4R)-ethyl 1-(3-(2-methoxyphenyl)oxetan-3-yl)-3-phenylpiperidine-4-carboxylate (274 ng, 0.692 mmol), lithium hydroxide monohydrate (87 mg, 2.078 mmol, 3.0 eq.) were stirred in ethanol (4 ml) and water (4 ml) at 45° C. for 20 hours. The mixture was partially evaporated and the aqueous residue was acidified with 1 N HCl (aq.). The resulted precipitate was filtered off, washed with water and dried to give Preparation R8g. HRMS calculated for C₂₂H₂₅NO₄: 367.1783; found 368.1855 ((M+H)⁺ form).

¹H-NMR (500 MHz, dmso-d6) δ ppm 12.33 (brs, 1H), 7.27 (t, 1H), 7.24 (t, 2H), 7.17 (d, 2H), 7.16 (t, 1H), 7.02 (d, 1H), 6.91 (t, 1H), 6.86 (d, 1H), 4.78/4.71/4.64 (d+d+d, 4H), 3.62 (s, 3H), 3.1/1.63 (d+t, 2H), 2.99 (td, 1H), 2.93/1.56 (d+t, 2H), 2.36 (td, 1H), 1.92/1.72 (ddd+ddd, 2H),

¹³C-NMR (500 MHz, dmso-d6) a ppm 176, 157.8, 143, 129.7, 129.3, 128.7, 128.2, 127, 125, 120.4, 112.3, 79.2/79.1, 65.8, 55.7, 536, 47.4, 46.1, 45.5, 30

### Preparation R8h: (3R,4R)-3-phenyl-1- 3-[3-(trifluoromethyl)phenyl]oxetan-3-yl]piperidine-4-carboxylic acid

### Step 1: (3R,4R)-ethyl 3-phenyl-1-(3-(3-(trifluormethyl)phenyl)oxetan-3-yl)piperidine-4-carboxylate

To a three-necked 100 ml flask under N₂ atmosphere, meta-trifluoromethyl-phenyl-MgBr solution (0.35M in abs. THF; 12 ml; 4.2 mmol; 6.0 eq.) was added via syringe. The Grignard solution was cooled to −10° C. with ice-NaCl cooling bath, and ZnCl₂ solution (2.0M in abs, 2-methyltetrahydrofuran; 1.1 ml; 2.24 mmol; 3.2 eq.) was added via syringe at such rate that the temperature is kept between −10° C. and −5° C. After stirring 5 minutes. Preparation R7b (0.2M abs. THF: 3.5 ml; 0.70 mmol; 1.0 eq.) was added dropwise to the organozinc reagent, keeping the temperature still between −10° C. and −5° C. The reaction mixture is allowed to warm to r.t. in 20 minutes then quenched with 5 mi sat. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂C₃ solution. Organic phase was dried over anhydrous Na₂SO₄ then evaporated to dryness. Purification by flash chromatography (4:1 hexanes/EtOAc) afforded (3R,4R)-ethyl 3-phenyl-1-(3-(3-(trifluoromethyl)phenyl)oxetan-3-yl)piperidine-4-carboxylate. HRMS calculated for C₂₄H₂₆F₃NO₃: 433.1865; found 434.1934 ((M+H)⁺ form).

¹H-NMR (500 MHz, dmso-d6) δ ppm 7.69 (dm, 1H), 7.62 (t, 1H), 7.52 (brs, 1H), 7.48 (dm, 1H), 7.27-7.13 (m, 5H), 4.81-4.71 (m, 4H), 3.81/3.77 (m+m, 2H), 3 (m, 1H), 2.99/1.58 (m+m, 2H), 2.88/1.52 (m+m, 2H), 2.48 (n, 1H), 1.89/1.75 (m+m, 2H), 0.85 (t, 3H).

¹³C-NMR (125 MHz, dmso-d6) δ ppm 132, 1293, 124.5, 124.2, 59.9, 52.9, 47.1, 45.8, 45.5, 29.4, 14.2

### Step 2: 1 Preparation R8h

(3R,4R)-ethyl 3-phenyl-1-(3-(3-(trifluoromethyl)phenyl)oxetan-3-yl)piperidine-4-carboxylate (235 mg, 0.542 mmol), lithium hydroxide monohydrate (68 mg, 1.626 mmol, 3.0 eq.) were stirred in ethanol (3 ml) and water (3 ml) at 45° C. for 18 hours. The mixture was partially evaporated and the aqueous residue was acidified with 1N HCl (aq.). The resulted precipitate was filtered off, washed with water and dried to give Preparation R8h. HRMS calculated for C₂₁H₂₂F₃NO₃: 405.1552; found 406.1624 ((M+H)⁺ form).

¹H-NMR (500 MHz, dmso-d6) δ ppm 11.94 (brs, 1H), 7.79-7.4 (m, 4H), 7.25 (t, 2H), 7.18 (d, 2H), 7.17 (t, 1H), 4.78 (brd, 4H), 3.01/1.56 (br.+br., 2H), 3 (br., 1H), 2.87/1.45 (br.+br., 2H), 2.41 (br., 1H), 1.94/1.75 (br.+br., 2H).

¹³C-NMR (125 MHz, dmso-d6) δ ppm 128.7, 128.3, 128.2, 79.7, 53, 47, 45.8, 45.4, 29.6

### Preparation R8i: (3R,4R)-1-[3-(3-fluorophenyl)oxetan-3-yl]-3-phenyl-piperidine-4-carboxylic acid

### Step 1: (3R,4R)-ethyl 1-(3-(3-fluorophenyl)oxetan-3-yl)-3-phenylpiperidine-4-carboxylate

To a three-necked 100 ml flask under N2 atmosphere, meta-fluoro-phenyl-MgBr solution (0.35M in abs. THF; 13.7 ml; 4.8 mmol: 6.0 eq.) was added via syringe. The Grignard solution was cooled to −10° C. with ice-NaCl cooling bath, and ZnCl₂ solution (2.0M in abs, 2-methyltetrahydrofuran; 1.3 ml; 2.6 mmol; 3.2 eq.) was added via syringe at such rate that the temperature is kept between −10° C. and −5° C. After stirring 5 minutes, Preparation R7b (0.2M abs. THF: 4.0 ml; 0.80 mmol; 1.0 eq.) was added dropwise to the organozinc reagent, keeping the temperature still between −10° C. and −5° C. The reaction mixture is allowed to warm to r.t. in 20 minutes then quenched with 5 ml sat. Na₂CO₃-solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂CO₃ solution. Organic phase was dried over anhydrous Na₂SO₄ then evaporated to dryness. Purification by flash chromatography (7:3 hexanes/EtOAc) afforded (3R,4R)-ethyl 1-(3-(3-fluorophenyl)oxetan-3-yl)-3-phenylpiperidine-4-carboxylate. HRMS calculated for C₂₃H₂₆FNO₃: 383.1897; found 384.198 ((M+H)⁺ form).

¹H-NMR (500 MHz, dmso-d6) δ ppm 7.41 (m, 1H), 7.28-7.13 (m, 5H), 7.14 (m, 1H), 7.07 (dm, 1H), 6.99 (dm, 1H), 4.79-4.66 (m, 4H), 3.81/3.78 (m+m, 2H), 3 (m, 1H), 2.94/1.63 (m+m, 2H), 2.83/1.55 (m+m, 2H), 2.48 (m, 1H), 1.89/1.74 (m+m, 2H), 0.85 (t, 3H).

¹³C-NMR (125 MHz, dmso-d6) δ ppm 130.1, 124, 114.8, 114.5, 59.9, 53, 47.2, 45.8, 45.6, 29.4, 14.3

### Step 2: Preparation R8i

(3R,4R)-ethyl 1-(3-(3-fluorophenyl)oxetan-3-yl)-3-phenylpiperidine-4-carboxylate (245 mg, 0.638 mmol), lithium hydroxide monohydrate (80 mg, 1.916 mmol, 3.0 eq.) were stirred in ethanol (3 ml) and water (3 ml) at 45° C. for 42 hours. The mixture was partially evaporated and the aqueous residue was acidified with 1N HCl (aq.). The resulted precipitate was filtered off, washed with water and dried to give Preparation R8i. HRMS calculated for C₂₁H₂₂FNO₃: 355.1584; found 356.1654 ((M+H)⁺ form).

¹H-NMR (500 MHz, dmso-d6) δ ppm 11.91 (brs, 1H), 7.41 (dd, 1H), 7.25 (t, 2H), 7.18 (d, 21), 7.16 (t, 1H), 7.15 (t, 1H), 7.07 (d, 1H), 6.99 (d, 1H), 4.77-4.68 (m, 4H), 3 (t, 1H), 2.93/1.61 (d+d, 2H), 2.8/1.49 (d+d, 2H), 2.39 (t, 1H), 1.93/1.73 (dd+dd, 2H).

¹³C-NMR (125 MHz, dmso-d6) δ ppm 130, 128.7, 128.3, 127, 124, 114.8, 114.5, 80/79.7, 53.3, 47, 45.8, 45.4, 29.7

### Preparation R8j: (3R,4R)-1-[3-(3-bromophenyl)oxetan-3-yl]-3-phenyl-piperidine-4-carboxylic acid

### Step 1: (3R,4R)-ethyl 1-(3-(3-bromophenyl)oxetan-3-yl]-3-phenylpiperidine-4-carboxylate

In a three-necked 100 ml flask under N₂ atmosphere, 1,3-dibromobenzene (0.93 ml; 7.7 mmol; 7 eq.) was dissolved in 8 ml abs. THF. The solution was cooled to −10° C. Then, i-PrMgCl-solution (1.3M in abs. THF; 5.1 ml; 6.6 mmol; 6.0 eq.) was added via syringe, while keeping the temperature under −5° C., The reaction mixture was stirred for 1 hour at −10° C. to give the solution of meta-bromo-phenyl-MgBr.

In a three-necked 100 ml flask under N_{T} atmosphere, a solution of meta-bromo-phenyl-MgBr (0.46M in abs. THF; 14 ml; 6.6 mmol; 6.0 eq.) was prepared. To the freshly prepared Grignard reagent, ZnCl₂ solution (2.0M in abs, 2-methyltetrahydrofuran; 1.76 ml; 3.52 mmol: 3.2 eq.) was added via syringe at such rate that the temperature is kept between −10° C. and −5′ C. After stirring 5 minutes, Preparation R7b (0.2M abs. THF: 5.5 ml: 1.1 mmol: 1.0 eq.) was added dropwise to the organozinc reagent, keeping the temperature still between −10° C. and −5° C. The reaction mixture is allowed to warm to r.t. in 20 minutes then quenched with 5 ml sat. Na₂CO₃ solution while keeping the temperature under 30° C. To the resulting suspension, 40 ml DCM was added, then stirred vigorously for 5 minutes. The slurry was filtered through a short pad of (Celite, diluted with 60 ml DCM then washed with 1) ml sat. Na₂CO₃ solution. Organic phase was dried over anhydrous Na₂SO₄ then evaporated to dryness. Purification by flash chromatography (75:25 hexanes/EtOAc) afforded (3R,4R)-ethyl 1-(3-(3-bromophenyl)oxetan-3-yl)-3-phenylpiperidine-4-carboxylate. HRMS calculated for C₂₃H₂₆BrNO₃: 443.1096; found 444.1167 ((M+H)⁺ form).

¹H-NMR (500 MHz, dmso-d6) δ ppm 7.51 (dm, 1H), 7.4 (t, 1H), 7.34 (t, 1H), 7.28-7.14 (m, 5H), 7.17 (dm, 1H), 4.78466 (m, 4H), 3.81/3.78 (m+m, 2H), 3 (m, 1H), 2.94/1.62 (m+m, 2H), 2.83/1.54 (m+m, 2H), 2.5 (in, 1H), 1.9/1.74 (m+m, 2H), 0.85 (t, 3H).

¹³C-NMR (125 MHz, dmso-d6) δ ppm 130.6, 130.4, 130.4, 127, 59.9, 53, 47.1, 45.8, 45.5, 29.4, 14.3

### Step 2: Preparation R8j

(3R,4R)-ethyl 1-(3-(3-bromophenyl)oxetan-3-yl)-3-phenylpiperidine-4-carboxylate (303 ng, 0.681 mmol), lithium hydroxide monohydrate (85 mg, 2.045 mmol, 3.0 eq.) were stirred in ethanol (3 ml) and water (3 ml) at 45° C. for 42 hours. The mixture was partially evaporated and the aqueous residue was acidified with 1 N HCl (aq.). The resulted precipitate was filtered off, washed with water and dried to give Preparation R8j. HRMS calculated for C₂₁H₂₂BrNO₃: 415.0783: found 416.0847 ((M+)⁺ form).

¹H-NMR (500 MHz, dmso-d6) δ ppm 11.9 (brs, 1H), 7.51 (d, 1H), 7.39 (s, 1H), 7.33 (t, 1H), 7.25 (t, 2H), 7.19 (d, 2H), 7.16 (t, 1H), 7.15 (d, 1H), 4.76-4.68 (m, 4H), 3 (t, 1H), 2.94/1.61 (d+t, 2H), 2.81/1.48 (d+t, 2H), 2.42 (t, 1H), 1.94/1.73 (d+dd, 2H).

¹³C-NMR (125 MHz, dmso-d6) δ ppm 130.6, 130.4, 130.3, 128.7, 128.3, 127, 127, 79.9/79.7, 53.2, 46.9, 45.8, 45.4, 29.7

### Preparation R8k: (3R,4R)-1-[3-(3-chlorophenyl)oxetan-3-yl]-3-phenyl-piperidine-4-carboxylic acid

### Step 1: (3R,4R)-ethyl 1-(3-(3-chlorophenyl)oxetan-3-yl)-3-phenylpiperidine-4-carboxylase

To a three-necked 100 ml flask under N₂ atmosphere, meta-chloro-phenyl-MgBr solution (0.42M in abs. THF; 15.7 ml: 6.6 mmol; 6.0 eq.) was added via syringe. The Grignard solution was cooled to −10° C. with ice-NaCl cooling bath, and ZnCl₂ solution (2.0M in abs, 2-methyltetrahydrofuran; 1.8 ml; 3.5 mmol: 3.2 eq.) was added via syringe at such rate that the temperature is kept between −10° C. and −5° C. After stirring 5 minutes. Preparation R7b (0.2M abs. THF: 5 ml: 1.1 mmol: 1.0 eq.) was added dropwise to the organozinc reagent, keeping the temperature still between −10° C. and −5° C. The reaction mixture is allowed to warm to r.t. in 20 minutes then quenched with 5 ml sat. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂CO₃ solution. Organic phase was dried over anhydrous Na₂SO₄ then evaporated to dryness. Purification by flash chromatography (7:3 hexanes/EtOAc) afforded (3R,4R)-ethyl 1-(3-(3-chlorophenyl)oxetan-3-yl)-3-phenylpiperidine-4-carboxylate. HRMS calculated for C₂₃H₂₆ClNO₃: 399.1601; Found 400.1672 ((M+H)⁺ form).

¹H-NMR (500 MHz, dmso-d6) δ ppm 7.43-7.09 (m, 9H), 4.79-4.65 (m, 4H), 3.81/3.78 (m+m, 2H), 3 (m, 1H), 2.94/1.62 (m+m, 21-1), 2.814/1.54 (m+m, 2H), 2.5 (m, 11-), 1.9/1.74 (m+m, 2H), 0.85 (t, 3H).

### ¹³C-NMR (125 MHz, dmso-d6) δ ppm 59.9, 53, 47.1, 45.8, 45.5, 29.4, 14.3

### Step 2: Preparation R8k

(3R,4R)-ethyl 1-(3-(3-chlorophenyl)oxetan-3-yl)-3-phenylpiperidine-4-carboxylate (353 mg, 0.882 mmol), lithium hydroxide monohydrate (111 mg, 2.648 mmol, 3.0 eq.) were stirred in ethanol (3 ml) and water (3 ml) at 45° C. for 42 hours. The mixture was partially evaporated and the aqueous residue was acidified with 1N HCl (aq.). The resulted precipitate was filtered off, washed with water and dried to give Preparation R8k, HRMS calculated for C₂₁H₂₂ClNO₃: 371.1288; found 372.1355 ((M+H)⁺ form).

¹H-NMR (500 MHz, dmso-d6) δ ppm 11.91 (brs, 1H), 7.39 (t, 1H), 7.38 (s, 1H), 7.26 (d, 1H), 7.25 (t, 2H), 7.19 (d, 2H), 7.17 (t, 1H), 7.12 (d, 1H), 4.8-4.67 (m, 4H), 3 (t, 1H), 2.94/1.6 (d+t, 2H), 2.81/1.48 (d+t, 2H), 2.41 (t, 1H), 1.94) 1.73 (d+dd, 21-),

### ¹³C-NMR (125 MHz, dmso-d6) δ ppm 130.1, 128.7, 128.3, 127.8, 127.7, 127.1, 126.6, 79.9/79.7, 53.2, 46.9, 45.8, 45.4, 29.7

### Preparation R8l: (3R,4R)-3-phenyl-1-[3-(p-tolyl)oxetan-3-yl]piperidine-4-carboxylic acid

### Step 1: (3R,4R)-ethyl 3-phenyl-1-(3-(p-tolyl)oxetan-3-yl)piperidin-4-carboxylate

To a three-necked 100 ml flask under N₂ atmosphere, para-methyl-phenyl-MgBr solution (0.50M in abs. THF; 15 ml; 7.5 mmol; 6.0 eq.) was added via syringe. The Grignard solution was cooled to −10° C. with ice-NaCl cooling bath, and ZnCl₂ solution (2.0M in abs, 2-methyltetrahydrofuran; 2.0 ml; 4.0 mmol; 3.2 eq.) was added via syringe at such rate that the temperature is kept between −10° C. and −5° C. After stirring 5 minutes, Preparation R7b (0.2M abs. THF; 6.3 ml; 1.25 mmol; 0.0 eq.) was added dropwise to the organozinc reagent, keeping the temperature still between −10° C. and −5° C. The reaction mixture is allowed to warm to r.t. in 20 minutes then quenched with 5 ml sat. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂CO₃ solution. Organic phase was dried over anhydrous Na₂SO₄ then evaporated to dryness. Purification by flash chromatography (7:3 hexanes/EtOAc) afforded (3R,4R)-ethyl 3-phenyl-1-(3-(p-tolyl)oxetan-3-yl)piperidine-4-carboxylate. HRMS calculated for C₂₄H₂₉NO₃: 379.2148; found 380.2219 ((M+H)⁺ form).

¹H-NMR (500 MHz, dmso-d6) δ ppm 7.27-7.13 (m, 5H), 7.17 (m, 2H), 7.02 (m, 2H), 4.78-4.65 (m, 4H), 3.82/3.78 (m+m, 2H), 3 (m, 1H) 2.91/1.61 (m+m, 2H), 2.78/1.54 (m+m, 2H), 2.44 (m, 1H), 2.31 (s, 3H), 1.88/1.74 (m+m, 2H), 0.85 (t, 3H).

¹³C-NMR (125 MHz, dmso-d6) δ ppm 128.7, 127.7, 59.9, 53.1, 47.2, 45.8, 45.6, 29.4, 21.2, 14.3

### Step 2: Prelparation R8l

(3R,4R)-Ethyl 3-phenyl-1-(3-(p-tolyl)oxetan-3-yl)piperidine-4-carboxylate (295 mg, 0.777 mmol), lithium hydroxide monohydrate (98 mg, 2.332 mmol, 3.0 eq.) were stirred in ethanol (4 ml) and water (4 ml) at 50° C. for 20 hours. The mixture was partially evaporated and the aqueous residue was acidified with 1N HCl (aq.). The resulted precipitate was filtered off, washed with water and dried to give Preparation R8l. HRMS calculated for C₂₂H₂₅NO₃: 351.1834; found 352.19090 ((M+H)⁺ form).

¹H-NMR (500 MHz, dmso-d6) δ ppm 12.41 (brs, 1H), 7.24 (1, 2H), 7.17 (t, 1H), 7.16 (d, 2H), 7.16 (d, 2H), 7.01 (d, 2H), 4.74/4.69 (d+d, 2H), 4.73 (s, 2H), 2.99 (td, 1H), 2.9/1.59 (d+t, 2H), 2.76/1.5 (d+t, 2H), 2.36 (td, 1H), 2.3 (s, 3H), 1.91/1.72 (ddd+ddd, 2H).

¹³C-NMR (125 MH, dmso-d6) δ ppm 175.8, 142.7, 136.6, 134.3, 128.7, 128.7, 128.2, 127.8, 127, 80.2, 80, 66.3, 53.4, 47, 45.8, 45.3, 29.7, 21.2

Preparation R8m: (3R,4R)-1-(3-(4-methoxyphenyl)oxetan-3-yl]-3-phenyl-piperidine-4-carboxylic acid

### Step 1: (3R,4R)-ethyl 1-(3-(4-methylphenyl)oxetan-3-yl)-3-phenylpiperidine-4-carboxylate

To a three-necked 100 mil flask under N2 atmosphere, para-methoxy-phenyl-MgBr solution (0.50M in abs. THF; 15 ml; 7.5 mmol; 6.0 eq.) was added via syringe. The Grignard solution was cooled to −10° C. with ice-NaCl cooling bath, and ZnCl₂ solution (2.0M in abs, 2-methyltetrahydrofuran; 2.0 ml; 4.0 mmol; 3.2 eq.) was added via syringe at such rate that the temperature is kept between −10° C. and −5° C. After stirring 5 minutes, Preparation R7b (0.2M abs. THF; 6.3 ml; 1.25 mmol; 1.0 eq.) was added dropwise to the organozinc reagent, keeping the temperature still between −10° C. and −5° C. The reaction mixture is allowed to warm to r.t. in 20 minutes then quenched with 5 ml sat, Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂CO₃ solution. Organic phase was dried over anhydrous Na₂SO₄ then evaporated to dryness. Purification by flash chromatography (7:3 hexanes/EtOAc) afforded (3R,4R)-ethyl 1-(3-(4-methoxyphenyl)oxetan-3-yl)-3-phenylpiperidine-4-carboxylate. HRMS calculated for C₂₄H₂₉NO₄: 395.2097; found 396.2173 ((M+H)⁺ form).

¹H-NMR (500 MHz, dmso-d6) δ ppm 7.27-7.13 (m, 5H), 7.07 (m, 2H), 6.91 (m, 2H), 4.77-4.65 (m, 4H), 3.82/3.77 (m+m, 2H), 3.75 (s, 3H), 3 (m, 1H), 2.89/1.61 (m+m, 2H), 2.77/1.55 (m+m, 2H), 2.45 (m, 1H), 1.88/1.73 (m+m, 2H), 0.86 (t, 3H).

¹³C-NMR (125 MHz, dmso-d6) δ ppm 129.1, 113.4, 59.9, 55.5, 53.1, 47.2, 45.8, 45.6, 29.4, 14.3

### Step 2: Preparation R8m

(3R,4R)-ethyl 3-phenyl-1-(3-(p-tolyl)oxetan-3-yl)piperidine-4-carboxylate (295 mg, 0.777 mmol), lithium hydroxide monohydrate (98 mg, 2.332 mmol, 3.0 eq.) were stirred in ethanol (4 ml) and water (4 ml) at 50° C. for 20 hours. The mixture was partially evaporated and the aqueous residue was acidified with 1N HCl (aq. The resulted precipitate was filtered off, washed with water and dried to give Preparation R8m. HRMS calculated for C₂₂H₂₅NO₄: 367.1783; found 368.1856 ((M+H)⁺ form).

¹H-NMR (500 MHz, dmso-d6) δ ppm 12.3 (brs, 1H), 7.24 (t, 2H), 7.17 (t, 1H), 7.17 (d, 2H), 7.06 (d, 2H), 6.9 (d, 2H), 4.73/4.68 (d+d, 2H), 4.72 (s, 2H), 3.75 (s, 3H), 2.99 (td, 1H), 2.89/1.59 (d+t, 2H), 2.75/1.48 (d+t, 2H), 2.36 (td, 1H), 1.92/1.72 (ddd+ddd, 2H).

¹³C-NMR (125 MHz, dmso-d6) δ ppm 175.8, 158.6, 142.7, 129.3, 129.1, 128.7, 128.2, 127, 13.5, 80.3, 80.1, 66.1, 55.5, 53.4, 47, 45.8, 45.4, 29.7

### Preparation R8n: (3R,4R)-1-[3-(2-chlorophenyl)oxetan-3-yl]-3-phenyl-piperidine-4-carboxylic acid

### Step 1: ethyl (3R,4R)-1-[3-(2-chlorophenyl)oxetan-3-yl]-3-phenyl-piperidine-4-carboxylate

In a three-necked 100 mil flask under N₂ atmosphere, 1-bromo-2-chlorobenzene (1.5 g; 7.7 mmol; 7 eq.) was dissolved in 8 ml abs. THF. The solution was cooled to −10° C. Then, i-PrMgCl.LiCl-solution (1.3M in abs. THF; 5 ml; 6.6 mmol; 6.0 eq.) was added via syringe, while keeping the temperature under −5° C. The Grignard-reagent was stirred between −10° C. and −5° C. for 1 hour to give the solution of ortho-chloro-phenyl-MgBr (0.5M in abs. THF; 13 ml; 6.6 mmol; 6.0 eq.). To the freshly prepared Grignard reagent, ZnCl₂ solution (2.0M in abs, 2-methyltetrahydrofuran: 1.76 ml; 3.52 mmol; 3.2 eq.) was added via syringe at such rate that the temperature is kept between −10° C. and −5° C. After stirring 5 minutes, Preparation R7b (0.2M abs. THF; 7.5 ml; 1.5 mmol; 1.0 eq.) was added dropwise to the organozinc reagent, keeping the temperature still between −10° C. and −5° C. The reaction mixture is allowed to warm to r.t. in 20 minutes then quenched with 3 ml sat. Na₂CO₃ solution while keeping the temperature under 30° C. To the resulting suspension, 40 ml DCM was added, then stirred vigorously for 5 minutes. The slurry was filtered through a short pad of Celite, diluted with 60 ml DCM then washed with 10 ml sat. Na₂CO₃-solution. Organic phase was dried over anhydrous Na₂SO₄ then evaporated to dryness. Purification by flash chromatography (70:30 hexanes/EtOAc) afforded ethyl (3R,4R)-1-[3-(2-chlorophenyl)oxetan-3-yl]-3-phenyl-piperidine-4-carboxylate. HRMS calculated for C₂₃H₂₆ClNO₃: 399.1601; found 400.1678 ((M+H)⁺ form).

¹H-NMR (5N) M Hz, dmso-d6) δ ppm 7.46-7.05 (m, 4H), 7.28-7.15 (in, 5H), 4.93-4.69 (m, 4H), 3.82/3.78 (m+m, 2H), 3.25/1.88 (m+m, 2H), 3.11/1.85 (m+m, 2H), 2.98 (m, 1H), 2.54 (m, 1H), 1.92/1.74 (m+m, 2H), 0.86 (t, 3H).

¹³C-NMR (125 MHI, dmso-d6) δ ppm 59.9, 53.4, 47.4, 46.1, 46, 29.6, 14.3

### Step 2: Preparation R8n

Ethyl (3R,4R)-1-[3-(2-chlorophenyl)oxetan-3-yl]-3-phenyl-piperidine-4-carboxylate (203 mg, 0.507 mmol), lithium hydroxide monohydrate (64 mg, 1.522 mmol, 3.0 eq.) were stirred in ethanol (4 mil) and water (4 ml) at 50° C. for 20 hours. The mixture was partially evaporated and the aqueous residue was acidified with 1N HCl (aq.). The solvent was evaporated, the resulted precipitate was purified by preparative LC (on C-18 Gemini-NX 5 μm column, 5 mM aqueous 0.2 w % HCOOH-MeCN, gradient) and evaporated to give Preparation R8m. HRMS calculated for C₂₁H₂₂ClNO₃: 371.1288; found 372.13610 ((M+H)+ form).

¹H-NMR (500 MHz, dmso-46) δ ppm 11.94 (brs, 1H), 7.42 (m, 1H), 7.32 (m, 1H), 7.32 (m, 1H), 7.25 (t, 2H), 7.2 (d, 2H), 7.17 (t, 1H), 7.07 (m, 1H), 4.9/4.73 (d+d, 2H), 4.84/4.74 (d+d, 2H), 3.23/1.86 (d+t, 2H), 3.09/1.78 (d+t, 2H), 2.97 (td, 1H), 2.45 (td, 1H), 1.96/1.71 (ddd+ddd, 2H).

¹³C-NMR (25 MHz, dmso-d6) δ ppm 175.9, 142.7, 135.1, 132.7, 131.2, 130.9, 129.8, 128.7, 128.2, 127.1, 127, 89, 77.9, 68.1, 53.7, 47.2, 46.3, 45.6, 30

### Preparation R9b: 1-[(3R,4R)-1-[3-(2-fluorophenyl)oxetan-3-yl]-3-phenyl-piperidine-4-carbonyl]piperidin-4-one

Using General Procedure 6 starting from Preparation R8b and 4-piperidone hydrochloride hydrate as reagents, Preparation R9b was obtained. HRMS calculated for C₂₆H₂₉FN₂O₃: 436.2162; found 437.22339 ((M+H)⁺ form).

¹H-NMR (500 MHz, dmso-d6) δ ppm 7.43-7.10 (m, 9H), 4.85-4.72 (m, 4H), 3.79-3.21 (m, 4H), 3.24-1.68 (m, 8H), 2.19-1.53 (n, 4H)

¹³C-NMR (125 MHz, dmso-d6) δ ppm 207.5, 172.8

### Preparation R9c: 1-[(3R,4R)-1- 3-(4-fluorophenyl)oxetan-3-yl]-3-phenyl-piperidine-4-carbonyl]piperidin-4-one

Using General Procedure 6 starting from Preparation R8e and 4-piperidone hydrochloride hydrate as reagents, Preparation R9c was obtained. HRMS calculated for C₂₆H₂₉FN₂O₃: 436.2162; found 437.2228 ((M+H)⁺ form),

¹H-NMR (500 MHz, dmso-d6) δ ppm 7.24-7.11 (m, 9H), 4.77-4.68 (m, 4H), 3.71/3.6613.51/3.28 (m+m+m+m, 4H), 3.13 (t, 1H), 2.99 (dd, 1H), 2.88/1.67 (t+d, 2H), 2.81/1.67 (dd+t, 2H), 2.1212.07/181/1.61 (m+m+m+m, 4H), 1.75/1.68 (m+m, 2H).

¹³C-NMR (125 MHz, dmso-d6) δ ppm 80.3/80, 52.5, 45.8, 45.6, 43.3/40.4, 42.8, 41.3/40.8, 29.7

### Preparation R9d: 1-[(3R,4R)-1-(3-methyloxetan-3-yl)-3-phenyl-piperidine-4-carbonyl]piperidin-4-one

Using General Procedure 6 starting from Preparation R8d and 4-piperidone hydrochloride hydrate as reagents, Preparation R9d was obtained. HRMS calculated for C₂₁H₂₈N₂O₃: 356.21: found 357,2159 ((M+H)⁻ form).

¹H-NMR (500 MHz, dmso-d6) δ ppm 7.25 (m, 2H), 7.25 (m, 2H), 7.15 (m, 1H), 4.42/4.41/4.12/4.07 (d+d+d+d, 4H), 3.75/3.72/3.5913.3 (n+m+m+m, 4H), 3.14 (t, 1H), 3.05 (t, 1H), 2.56/2.25 (d+t, 2H), 2.47/2.29 (di, 2H), 2.16/2.14/1.85/1.63 (m+m+m+m, 4H), 1.78/1.73 (t+t, 2H), 1.28 (s, 3H).

¹³C-NMR (125 MHz, dmso-d6) δ ppm 207.5, 172.9, 143.1, 128.7, 128.3, 127, 81.3/81.2, 60.2, 51.8, 45.8, 44.8, 43.4/40.5, 43.2, 41.2/40.8, 29.9, 14.7

### Preparation R9e: 1-[(3R,4R)-1-(3-isopropyloxetan-3-yl)-3-phenyl-piperidine-4-carbonyl]piperidin-4-one

Using General Procedure 6 starting from Preparation R8e and 4-piperidone hydrochloride hydrate as reagents, Preparation R9e was obtained. HRMS calculated for C₂₉H₃₂N₂O₃: 384,2413; found 385.2485 ((M+H)⁺ form).

¹H-NMR (500 MHz, dmso-d6) δ ppm 7.25 (m, 2H), 7.25 (m, 24), 7.16 (m, 1H), 4.52/4.49/4.25/4.24 (d+d+d+d, 4H), 3.79-1.60 (m, 14H), 3.19 (t, 1H), 2.98 (t, 1H), 2.11 (m, 1H), 0.99 (d, 6H)

¹³C-NMR (125 MHz, dmso-d6) δ ppm 207.6, 173, 143.2, 128.7, 128.3, 127, 74.7/74.6, 66.2, 46.6, 43.5, 31.2, 18.5/18.4

### Preparation R10b: [(3R,4R)-1-[3-(2-fluorophenyl)oxetan-3-yl]-3-phenyl-4-piperidyl]-(1-oxa-6-azaspiro[2.5]octan-6-yl)methanone

Preparation R9b (180 mg, 0.412 mmol) and trimethylsulfoxonium-iodide (1.031 mmol, 2.5 eq.) was stirred in MeCN (8 ml) and MTBE (8 ml) at r.t. and solution of NaOH (1.031 mmol, 2.5 eq.) in water (1.1 ml) was added to the mixture. Then the reaction mixture was stirred at 60° C. for 21 hours. The reaction mixture was evaporated under reduced pressure. It was dissolved in 8 ml DCM and 5 ml water. The layers were separated and the aqueous phase was extracted with 2×8 ml DCM. The combined organic layer was dried over MgSO₄ evaporated under reduced pressure to give Preparation R10b. HRMS calculated for C₂₇H₃₁FN₂O₃: 450.2319; found 451.2384 ((M+H)⁺ form).

¹H-NMR (500 MHz, dmso-d6) δ ppm 7.38 (m, 1H), 7.23 (t, 2H), 7.22 (td, 1H), 7.2 (m, 1H), 7.17 (t, 1H), 7.16 (d, 2H), 7.14 (dd, 1H), 4.81/4.74 (d+d, 4H), 4.75 (s, 4H), 3.74-0.89 (n, 14H), 3.14 (t, 1H), 3 (m, 1H), 2.56/2.52 (t+dd, 2H)

¹³C-NMR (125 MHz, dmso-d6) δ ppm 130.7, 130.1, 128.6, 128.3, 126.9, 124.4, 116.2, 79.3, 79.1, 53.4/53.1, 45.7, 42.5/42.4

### Preparation R10c: 1(3R,4R)-1-{3-(4-fluorophenyl)oxetan-3-yl]-3-phenyl-4-piperidyl]-(1-oxa-6-azaspiro(2.5]octan-6-yl)methanone

Preparation R9c (68 mg, 0.156 mmol) and trimethylsulfoxonium-iodide (0.389 mmol, 2.5 eq.) was stirred in MeCN (3 ml) and MTBE (3 mil) at r.t. and solution of NaOH (0.389 mmol, 2.5 eq.) in water (0.4 ml) was added to the mixture. Then the reaction mixture was stirred at 60° C. for 19 hours. The reaction mixture was evaporated under reduced pressure. It was dissolved in 3 ml DCM and 2 ml water. The layers were separated and the aqueous phase was extracted with 2×3 ml DCM. The combined organic layer was dried over MgSO₄ evaporated under reduced pressure to give Preparation R10c. FIRMS calculated for C₂₇H₃₁FN₂O₃: 450.2319: found 451.2396 ((M+H)⁺ form).

¹H-NMR (500 MHz, dmso-d6) δ ppm 7.28-7.10 (n, 9H), 4.79/4.66 (s, 4H), 3.77-2.88 (m, 6H), 3, 18-1.57 (m, 6H), 2.60-2.48 (m, 2H), 1.37-0.87 (m, 4H),

### Preparation R10d: [(3R,4R)-1-(3-methyloxetan-3-yl)-3-phenyl-4-piperidyl]-(1-oxa-6-azaspiro[2.5]octan-6-yl)methanone

Preparation R9d (14 mg, 0.039 mmol) and trimethylsulfoxonium-iodide (0.098 mmol, 2.5 eq.) was stirred in MeCN (2 ml) and MTBE (2 ml) at r.t and solution of NaOH (0.098 mmol, 2.5 eq.) in water (0.2 ml) was added to the mixture. Then the reaction mixture was stirred at 60° C. for 19 hours. The reaction mixture was evaporated under reduced pressure. It was dissolved in 2 ml DCM and 2 ml water. The layers were separated and the aqueous phase was extracted with 2×2 ml DCM. The combined organic layer was dried over MgSO₄ evaporated under reduced pressure to give Preparation R10d. HRMS calculated for C₂₂H₃₀N₂O₃: 370.2256; found 371.2340 ((M+H)⁺ form).

¹H-NMR (50(0 MHz, dmso-d6) δ ppm 7.30-7.12 (m, 5H), 4.44-4.04 (d+d, 4H), 3.81-2.97 (m, 6H), 2.64-2.50 (m, 2H), 2.62-2.18 (m, 4H), 1.80-1.62 (m, 2H), 1.44-0.89 (m, 4H), 1.27 (s, 3H)

### Preparation R10e: (3R,4R)-1-(3-isopropyloxetan-3-yl)-3-phenyl-4-piperidyl]-(1-oxa-6-azaspiro[2.5]octan-6-yl)methanone

Preparation R9e (30 mg, 0.078 mmol) and trimethylsulfoxonium-iodide (0.195 mmol, 2.5 eq.) was stirred in MeCN (2 ml) and MTBE (2 mi) at r.t. and solution of NaOH (0.195 mmol, 2.5 eq.) in water (0.2 ml) was added to the mixture. Then the reaction mixture was stirred at 60° C. for 18 hours. The reaction mixture was evaporated under reduced pressure. It was dissolved in 3 ml DCM and 3 ml water. The layers were separated and the aqueous phase was extracted with 2×3 ml DCM. The combined organic layer was dried over MgSO₄ evaporated under reduced pressure to give Preparation R10e. HRMS calculated for C₂₄H₃₄N₂O₃: 398.257; found 399.2646 ((M+H)⁺ form).

¹H-NMR (400 MHz, dmso-d6) δ ppm 7.40-7.05 (m, 5H), 4.55/4.18 (m, 4H), 3.90-2.50 (m, 12H), 2.1 (m, 1H), 1.80-0.90 (m, 6H), 0.99 (d, 6H)

### Preparation R11a: 5-amino-3-[[(3S,4S)-3-(fluoro-4-hydroxy-4-piperidyl]methyl-6-(4-fluorophenoxy)pyrimidin-4-one and 5-amino-3-[[(3R,4R)-3-fluoro-4-hydroxy-4-piperidyl]methyl]-6-(4-fluorophenoxy)pyrimidin-4-one

### and

### Preparation R11b: 5-amino-3-[[(3R,4S)-3-fluoro-4-hydroxy-4-piperidyl]methyl-6(4-fluorophenoxy)pyrimidin-4-one and 5-amino-3-[[(3S,4R)-3-fluoro-4-hydroxy-4-piperidyl]methyl]-6-(4-fluorophenoxy)pyrimidin-4-one

The sodium hydroxide (216 ng, 2.5 eq., 54088 mmol) was dissolved in water (0.4 mL) and added to the stirred mixture of tert-butyl 3-fluoro-4-oxo-piperidine-1-carboxylate (470 mg, 2.1635 mmol), trimethyl sulfoxonium, iodide (1:1) (2.5 eq., 5.4088 mmol) dissolved in acetonitrile (3 mL) and 2-methoxy-2-methyl-propane (3 mL). Then the mixture was heated at 60° C. for 2 hours. Then potassium carbonate (598 mg, 2 eq., 4.327 mmol) and Preparation R4h (649 mg, 1 eq., 2.163 mmol) was added to this mixture and stirred at 70° C. for 17 hours. Then the reaction mixture was evaporated. The residue was purified by preparative LC (on C-18 Gemini-NX 5 μm column, 5 mM aqueous NH₄HCO₃-MeCN, gradient) and evaporated. Then the product was purified again by preparative LC (on C-18 Gemini-NX 5 μm column, 5 mM aqueous NH₄HCO₃-MeCN, 48% (ACN) isocratic method) and evaporated to give:
the mixture of tert-butyl (3S,4S)-4-[[5-amino-4-(4-fluorophenoxy)-6-oxo-pyrimidin-1-yl]methyl]-3-fluoro-4-hydroxy-piperidine-1-carboxylate and tert-butyl (3R,4R)-4-115-amino-4-(4-fluorophenoxy)-6-oxo-pyrimidin-1-yl]methyl]-3-fluoro-4-hydroxy-piperidine-1-carboxylate), and
the mixture of tert-butyl (3S,4R)-4-[[5-amino-4-(4-fluorophenoxy)-6-oxo-pyrimidin-1-yl]methyl]-3-fluoro-4-hydroxy-piperidine-1-carboxylate and tert-butyl (3R,4S)-4-[[5-amino-4-(4-fluorophenoxy)-6-oxo-pyrimidin-1-yl]methyl]-3-fluoro-4-hydroxy-piperidine-1-carboxylate).

The mixture of tert-butyl (3S,4S)-4-[[5-amino-4-(4-fluorophenoxy)-6-oxo-pyrimidin-1-yl]methyl]-3-fluoro-4-hydroxy-piperidine-1-carboxylate and tert-butyl (3R,4R)-4-[[5-amino-4-(4-fluorophenoxy)-6-oxo-pyrimidin-1-yl]methyl]-3-fluoro-4-hydroxy-piperidine-1-carboxylate) were reacted using General procedure 5 to give Preparation R11a. HRMS calculated for C₁₆H₁₈F₂N₄O₃: 352.1347: found 353.1413 ((M+H)⁺ form).

¹H-NMR (500 MHz, dmso-d6) δ ppm 7.69 (s, 1H), 7.2 (t, 2H), 7.09 (dd, 2H), 5.12 (s, 1H), 4.67 (s, 2H), 4.41 (ddd, 1H), 4.2/3.98 (d+d, 2H), 2.87/2.78 (m+m, 2H), 2.55 (m, 2H), 1.42/1.33 (m+m, 2H).

¹³C-NMR (125 MH, dmso-d6) δ ppm 159.2, 158.8, 151, 146.8, 139.2, 121.8, 120, 116.4, 92, 71.7, 51.6, 45.5, 40.8, 35.

The mixture of tert-butyl (3S,4R)-4-[[5-amino-4-(4-fluorophenoxy)-6-oxo-pyrimidin-1-yl]methyl]-3-fluoro-4-hydroxy-piperidine-1-carboxylate and tert-butyl (3R,4S)-4-[(5-amino-4-(4-fluorophenoxy)-6-oxo-pyrimidin-1-yl]methyl]-3-fluoro-4-hydroxy-piperidine-1-carboxylate) were reacted using General procedure 5 to give the mixture of Preparation R11b. HRMS calculated for C₁₆H₁₈F₂N₄O₃: 352.1347; found 353.1418 ((M+H)⁺ form).

¹H-NMR (500 MHz, dmso-d6) δ ppm 7.65 (s, 1H), 7.2 (t, 2H), 7.09 (dd, 2H), 5.33 (s, 1H), 4.69 (s, 2H), 4.38/3.75 (dd+d, 2H), 4.19 (dd, 1H), 2.94/2.85 (td+dd, 2H), 2.64 (m, 2H), 1.92 (br., 2H), 1.59/1.14 (id+dt, 2H).

¹³C-NMR (125 MHz, dmso-d6) δ ppm 159.3, 158.8, 151, 146.8, 139.3, 121.7, 120.2, 116.4, 90.6, 70.5, 51.4, 46, 40.8, 32.2.

### EXAMPLES

The following Examples illustrate the invention but do not limit it in any way.

### tert-butyl (3R,4R)-4-[4-[[5-amino-4-(4-chlorophenoxy)-6-oxo-pyrimidin-1-yl]methyl]-4-hydroxy-piperidine-1-carbonyl]-3-phenyl-piperidine-1-carboxylate (Example 1)

Using General Procedure 4 starting from Preparation R4a and Preparation R5a as reagents, EXAMPLE 1 was obtained. HRMS calculated for C₃₃H₄₀ClN₅O₆: 637.2667: found 638.2738 ((M+H)⁺ form).

### tert-butyl (3R,4R)-4-[4-[[5-amino-4-(3-chloro-5-methoxy-phenoxy)-6-oxo-pyrimidin-1-yl]methyl]-4-hydroxy-piperidine-1-carbonyl]-3-phenyl-piperidine-1-carboxylate (Example 2)

Using General Procedure 4 starting from Preparation R4b and Preparation R8a as reagents, EXAMPLE 2 was obtained. HRMS calculated for C₃₄H₄₂ClN₅O₇: 667.2773; found 668.286 (M+H)⁺ form).

### tert-butyl (3R,4R)-4-[4-[[5-amino-4-(4-chloro-3-fluoro-phenoxy)-6-oxo-pyrimidin-1-yl]methyl]-4-hydroxy-piperidine-1-carbonyl]-3-phenyl-piperidine-1-carboxylate (Example 3)

Using General Procedure 4 starting from Preparation R4c and Preparation R5a as reagents, EXAMPLE 3 was obtained. HRMS calculated for C₃₃H₃₉ClFN₅O₆: 655.2573; found 678.2461 (M+Na)⁺ form).

### tert-butyl (3R,4R)-4-[4-[[5-amino-4(4-fluoro-3-methoxy-phenoxy)-6-oxo-pyrimidin-1-yl]methyl]-4-hydroxy-piperidine-1-carbonyl]-3-phenyl-piperidine-1-carboxylate (Example 4)

Using General Procedure 4 starting from Preparation R4d and Preparation R5a as reagents, EXAMPLE 4 was obtained. HRMS calculated for C₃₄H₄₂FN₅O₇: 651.3068: found 652.3146 ((M+H)⁺ form).

### tert-butyl (3R,4R)-4-[4-[[5-amino-6-oxo-4-[3-(trifluoromethyl)phenoxy]pyrimidin-1-yl]methyl]-4-hydroxy-piperidine-1-carbonyl]-3-phenyl-piperidine-1-carboxylate (Example 5)

Using General Procedure 4 starting from Preparation R4e and Preparation R5a as reagents, EXAMPLE 5 was obtained. HRMS calculated for C₃₄H₄₀F₃N₅O₆: 671.2931; found 672.2993 ((M+H)⁺ form).

### tert-butyl (3R,4R)-4-[4-[[5-amino-6-oxo-4-[3-(trifluoromethoxy)phenoxypyrimidin-1-yl]methyl]-4-hydroxy-piperidine-1-carbonyl]-3-phenyl-piperidine-1-carboxylate (Example 6)

Using General Procedure 4 starting from Preparation R4f and Preparation R5a as reagents, EXAMPLE 6 was obtained. HRMS calculated for C₃₄H₄₀FN₅O₇: 687.288; found 688.2958 ((M+H)⁺ form).

### tert-butyl (3R,4R)-4-[4-[(5-amino-6-oxo-4-phenoxy-pyrimidin-1-yl)methyl]-4-hydroxy-piperidine-1-carbonyl]-3-phenyl-piperidine-1-carboxylate (Example 7)

Using General Procedure 4 starting from Preparation R4g and Preparation R8a as reagents, EXAMPLE 7 was obtained. HRMS calculated for C₃₃H₄₀N₅O₆: 603.3057; found 604.314 ((M+H)⁺ form).

### tert-butyl (3R,4R)-4-[4-[[5-amino-4-(4-fluorophenoxy)-6-oxo-pyrimidin-1-yl]methyl]-4-hydroxy-piperidine-1-carbonyl]-3-phenyl-piperidine-1-carboxylate (Example 8)

Using General Procedure 4 starting from Preparation R4h and Preparation R5a as reagents, EXAMPLE 8 was obtained. HRMS calculated for C₃₃H₄₀FN₅O₆: 621.2963; found 622.3033 ((M+H)⁺ form).

### tert-butyl (3R,4R)-4-[(4S)-4-[(5-amino-6-oxo-4-phenoxy-pyrimidin-1-yl)methyl]-3,3-difluoro-4-hydroxy-piperidine-1-carbonyl]-3-phenyl-piperidine-1-carboxylate (Example 9)

Using General Procedure 4 starting from Preparation R4g and Preparation R5b as reagents, the enantiomers were separated by chiral chromatography to give EXAMPLE 9. HRMS calculated for C₃₃H₃₉F₂N₅O₆: 639.2869: found 662.2762 ((M+Na)⁺ form).

### tert-butyl (3R,4R)-4-(4S)-4-[[5-amino-4-(4-fluorophenoxy)-6-oxo-pyrimidin-1-yl]methyl]-3,3-difluoro-4-hydroxy-piperidine-1-carbonyl]-3-phenyl-piperidine-1-carboxylate (Example 10)

Using General Procedure 4 starting from Preparation R4h and Preparation R5b as reagents, the enantiomers were separated by chiral chromatography to give EXAMPLE 10. HRMS calculated for C₃₃H₃₈F₃N₅O₆: 657,2774; found 680,2659 ((M+Na)⁺ form),

### 5-amino-3-[[(4S)-3,3-difluoro-4-hydroxy-1-[(3R,4R)-3-phenylpiperidine-4-carbonyl]-4-piperidyl]methyl]-6-(4-fluorophenoxy)pyrimidin-4-one (Example 11)

Using General Procedure 5 starting from EXAMPLE 10 as reagent, the crude product was purified by preparative LC (on C-18 Luna 10 μm column, 5 mM aqueous NH₄HCO₃-MeCN, gradient). Solvent was evaporated under reduced pressure to give EXAMPLE 11. HRMS calculated for C₂₈H₃₀F₃N₅O₄: 557.225; found 558.2314 ((M+H)⁺ form).

### 5-amino-3-[[(4S)-3,3-difluoro-4-hydroxy-1-[(3R,4R)-3-phenylpiperidine-4-carbonyl]-4-piperidyl]methyl]-6-phenoxy-pyrimidin-4-one (Example 12)

Using General Procedure 5 starting from EXAMPLE 9 as reagent, the crude product was purified by preparative LC (on C-18 Gemini-NX 5 μm column, 5 mM aqueous NH₄HCO₃-MeCN, gradient). Solvent was evaporated under reduced pressure to give EXAMPLE 12. HRMS calculated for C₂₈H₃₁F₂N₅O₄: 539.2344; found 540.2406 (M+H)⁺ form).

### 5-amino-3-{[(4S)-3,3-difluoro-4-hydroxy-1-{(3R,4R)-1-[(2-methylpyrimidin-4-yl)methyl]-3-phenylpiperidine-4-carbonyl]piperidin-4-yl]methyl)-6-(4-fluorophenoxy)pyrimidin-4(31)-one (Example 13)

Using General Procedure 9 starting from EXAMPLE 11 and 4-(chloromethyl)-2-methyl-pyrimidine as reagents, EXAMPLE 13 was obtained. HRMS calculated for C₃₄H₃₆F₃N₇O₄: 663.2781; found 664.2849 ((M+H)⁺ form),

### 5-amino-6-(4-chlorophenoxy)-3-[[4-hydroxy-1-[(3R,4R)-3-phenylpiperidine-4-carbonyl]-4-piperidyl]methyl]pyrimidin-4-one hydrochloride (Example 14)

Using General Procedure 5 starting from EXAMPLE 1 as reagent, the crude product was purified by preparative LC (on C-18 Gemini-NX 5 μm column, 5 mM aqueous NH₄HCO₃-MeCN, gradient). Solvent was evaporated under reduced pressure to give EXAMPLE 14. HRMS calculated for C₂₈H₃₂ClN₅O₄: 537.2143; found 538.2222 ((M+H)⁺ form).

### 5-amino-6-(3-chloro-5-methoxy-phenoxy)-3-[[4-hydroxy-1-[(3R,4R)-3-phenylpiperidine-4-carbonyl]-4-piperidyl]methylpyrimidin-4-one (Example 15)

Using General Procedure 5 starting from EXAMPLE 2 as reagent, the crude product was purified by preparative LC (on C-18 Gemini-NX 5 μm column, 5 mM aqueous NH₄HCO₃-MeCN, gradient), Solvent was evaporated under reduced pressure to give EXAMPLE 15, HRMS calculated for C₂₉H₃₄ClN₅O₅: 567.2249; found 568.232 ((M+H)⁺ form).

### 5-amino-6-(4-chloro-3-fluoro-phenoxy)-3-[[4-hydroxy-1-{(3R,4R)-3-phenylpiperidine-4-carbonyl]-4-piperidyl]methylpyrimidin-4-one (Example 16)

Using General Procedure 5 starting from EXAMPLE 3 as reagent, the crude product was purified by preparative LC (on C-18 Gemini-NX 5 μm column, 5 mM aqueous NH₄HCO₃-MeCN, gradient). Solvent was evaporated under reduced pressure to give EXAMPLE 16. HRMS calculated for C₁₆H₃₁ClFN₅O₄: 555.2048; found 556.2118 ((M+H)⁺ form).

### 5-amino-6-(4-fluoro-3-methoxy-phenoxy)-3-[[4-hydroxy-1-(3R,4R)-3-phenylpiperidine-4-carbonyl]-4-piperidyl]methyl]pyrimidin-4-one hydrochloride (Example 17)

Using General Procedure 5 starting from EXAMPLE 4 as reagent, the crude product was purified by preparative LC (on C-18 Gemini-NX 5 μm column, 5 mM aqueous NH₄HCO₃-MeCN, gradient), Solvent was evaporated under reduced pressure to give EXAMPLE 17. HRMS calculated for C₂₉H₃₄FN₅O₅: 551.2544; found 552.2614 ((M+H)⁺ form).

### 5-amino-3-[[4-hydroxy-1-[(3R,4R)-3-phenylpiperidine-4-carbonyl]-4-piperidyl]methyl]-6-[3-(trifluoromethyl)phenoxy]pyrimidin-4-one hydrochloride (Example 18)

Using General Procedure 5 starting from EXAMPLE 5 as reagent, EXAMPLE 18 was obtained. HRMS calculated for C₂₉H₃₂F₃N₅O₄: 571.2407; found 572.2466 ((M+H)⁺ form).

### 5-amino-3-[[4-hydroxy-1-[(3R,4R)-3-phenylpiperidine-4-carbonyl]-4-piperidyl]methyl]-6-[3-(trifluoromethoxy)phenoxy]pyrimidin-4-one hydrochloride (Example 19)

Using General Procedure 5 starting from EXAMPLE 6 as reagent, EXAMPLE 19 was obtained. HRMS calculated for C₂₉H₃₂F₃N₅O₅: 587.2355: found 588.2414 ((M+H)⁺ form).

### 5-amino-3-[4-hydroxy-1-[(3R,4R)N-phenylpiperidine-4-carbonyl-4-piperidyl]methyl]-6-phenoxy-pyrimidin-4-one (Example 20)

Using General Procedure 5 starting from EXAMPLE 7 as reagent, the crude product was purified by preparative LC (on C-18 Gemini-NX 5 μm column, 5 mM aqueous NH₄HCO₃-MeCN, gradient). Solvent was evaporated under reduced pressure to give EXAMPLE 20. HRMS calculated for C₂₈H₃₃N₅O₄: 503.2533: found 504.2602 ((M+H)⁺ form).

### 5-amino-6-(4-fluorophenoxy)-3-[[4-hydroxy-1-[(3R,4R)-3-phenylpiperidine-4-carbonyl]-4-piperidyl]methyl]pyrimidin-4-one (Example 21)

Using General Procedure 5 starting from EXAMPLE 8 as reagent, the crude product was purified by preparative LC (on C-18 Luna 10 μm column, 5 mM aqueous NH₄HCO₃-MeCN, gradient). Solvent was evaporated under reduced pressure to give EXAMPLE 21 was obtained. HRMS calculated for C₂₈H₃₂FN₅O₄: 521.2438; found 522,2499 ((M+H)⁺ form).

### 5-amino-6-(4-chlorophenoxy)-3-[(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one (Example 22)

Using General Procedure 6 starting from EXAMPLE 14 and 4-methyl-2-(6-methyl-3-pyridyl)thiazole-5-carboxylic acid as reagents, EXAMPLE 22 was obtained. HRMS calculated for C₃₉H₄₀ClN₇O₅S: 753.25: found 754.2583 ((M+H)⁺ form).

### 5-amino-6-(3-chloro-5-methoxyphenoxy)-3-((4-hydroxy-1-((3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3/I)-one (Example 23)

Using General Procedure 6 starting from EXAMPLE 15 and 4-methyl-2-(6-methyl-3-pyridyl)thiazole-5-carboxylic acid as reagents, EXAMPLE 23 was obtained. HRMS calculated for C₄₀H₄₂ClN₇O₆S: 783.2606; found 784.2664 ((M+H)⁺ form).

### 5-amino-6-(4-chloro-3-fluorophenoxy)-3-[(4-hydroxy-1-{(3R,4R)-1-[4-methy-2-(6-methylpyridin-3-yl)-1,3-thiazole-5-carbonyl]-3-phenylpiperidine-4-carbonyl}piperidin-4-yl)methyl]pyrimidin-4(3H)-one (Example 24)

Using General Procedure 6 starting from EXAMPLE 16 and 4-methyl-2-(6-methyl-3-pyridyl)thiazole-5-carboxylic acid as reagents, EXAMPLE 24 was obtained. HRMS calculated for C₃₉H₃₉ClFN₇O₅S: 771.2406; found 772.2473 ((M+H)⁺ form).

### 5-amino-6-(4-fluoro-3-methoxyphenoxy)-3-[(4-hydroxy-1-{(3R,4R)-1-[4-methyl-2-(6-methylpyridin-3-yl)-1,3-thiazole-5-carbonyl]3-phenylpiperidine-4-carbonyl)piperidin-4-yl)methylpyrimidin-4(3H)-one (Example 25)

Using General Procedure 6 starting from EXAMPLE 17 and 4-methyl-2-(6-methyl-3-pyridyl)thiazole-5-carboxylic acid as reagents, EXAMPLE 25 was obtained. HRMS calculated for C₄₀H₄₂FN₇O₆S: 767.2902; found 768.2968 ((M+H)⁺ form).

### 5-amino-3-[(4-hydroxy-1-{(3R,4R)-1-[4-methyl-2-(6-methylpyridin-3-yl)-1,3-thiazole-5carbonyl]-3-phenylpiperidine-4-carbonyl}piperidin-4-yl)methyl-6-[3-(trifluoromethyl)phenoxy]pyrimidin-4(3H)-one (Example 26)

Using General Procedure 6 starting from EXAMPLE 18 and 4-methyl-2-(6-methyl-3-pyridyl)thiazole-5-carboxylic acid as reagents, EXAMPLE 26 was obtained. HRMS calculated for C₄₀H₄₀F₃N₇O₅S: 787.2764; found 788.284 ((M+H)⁺ form).

### 5-amino-3-[(4-hydroxy-1-((3R,4R)-1-[4-methyl-2-(6-methylpyridin-3-yl)-1,3-thiazole-5-carbonyl]-3-phenylpiperidine-4carbonyl}piperidin-4-yl)methyl]-6-[3-(trifluoromethoxy)phenoxy]pyrimidin-4(3H)-one (Example 27)

Using General Procedure 6 starting from EXAMPLE 19 and 4-methyl-2-(6-methyl-3-pyridyl)thiazole-5-carboxylic acid as reagents, EXAMPLE 27 was obtained. HRMS calculated for C₄₀H₄₀F₃N₇O₆S: 803.2713; found 804.27833 ((M+H)⁺ form).

### 5-amino-6-(4-fluorophenoxy)-3-[[4-hydroxy-1-[(3R,4R)-3-phenyl-1-thiazole-4-carbonyl)piperidine-4-carbonyl-4-piperidyl]methyl]pyrimidin-4-one (Example 28)

Using General Procedure 6 starting from Example 21 and thiazole-4-carboxylic acid as reagents, Example 28 was obtained. HRMS calculated for C₃₂H₃₃FN₆O₅S: 632.2217; found 633.2285 ((M+H)⁺ form).

### 5-amino-3-({1-[(3R,4R)-1-(2-bromo-4-methyl-1,3-thiazole-5-carbonyl)-3-phenylpiperidine-4-carbonyl]-4-hydroxypiperidin-4-yl}methyl)-6-(4-fluorophenoxy)pyrimidin-4(3H)-one (Example 29)

Using General Procedure 6 starting from EXAMPLE 21 and 2-bromo-4-methyl-thiazole-5-carboxylic acid as reagents, EXAMPLE 29 was obtained. HRMS calculated for C₃₃H₃₄BrFN₆O₅S: 724,1479; found 725-1548 ((M+H)⁺ form).

### 5-amino-3-({1-[(3R,4R)-1-(benzenesulfonyl)-3-phenyl-piperidine-4-carbonyl]A-hydroxy-4-piperidyl]methyl]-6-(4-fluorophenoxy)pyrimidin-4-one (Example 30)

Using General Procedure 9 starting from EXAMPLE 21 and benzenesulfonyl chloride as reagents, EXAMPLE 30 was obtained. HRMS calculated for C₃₄H₃₆FN₅O₆S: 661.2371; round 662.2443 ((M+H)⁺ form).

### 5-amino-3-({1-((3R,4R)-1-(3-bromobenzoyl)-3-phenylpiperidine-4carbonyl]-4-hydroxypiperidin-4-yl)methyl)-6-(4-fluorophenoxy)pyrimidin-4(3H)-one (Example 31)

Using General Procedure 6 starting from EXAMPLE 21 and 3-bromobenzoic acid as reagents, EXAMPLE 31 was obtained. HRMS calculated for C₃₅H₃₅BrFN₅O₅: 703.1805; found 704.1875 ((M+H)⁺ form).

### 5-amino-6-(4-fluorophenoxy)-3-[[4-hydroxy-1-{(3R,4R)-1-{3-(6-methyl-3-pyridyl) benzoyl]-3-phenyl-piperidine-4-carbonyl-4-piperidyl]methyl]pyrimidin-4-one (Example 32)

Using General Procedure 11 starting from EXAMPLE 31 and (6-methyl-3-pyridyl)boronic acid as reagents. EXAMPLE 32 was obtained. HRMS calculated for C₄₁H₄₁FN₆O₅: 716.3123; found 717.3189 ((M+H)⁺ form).

### 5-amino-3-({1-[(3R,4R)-1-(5-bromopyridine-3-carbonyl)-3-phenylpiperidine-4-carbonyl]-4-hydroxypiperidin-4-yl}methyl)-6-(4-fluorophenoxy)pyrimidin-4(3)-one (Example 33)

Using General Procedure 6 starting from EXAMPLE 21 and 5-bromopyridine-3-carboxylic acid as reagents. EXAMPLE 33 was obtained. HRMS calculated for C₃₄H₃₄BrFN₆O₅: 704.1758: found 705.1831 ((M+H)⁺ form).

### 5-amino-6-(4-fluorophenoxy)-3-({4-hydroxy-1-[(3R,4R)-1-(6′-methyl[3,3′-bipyridine]-5-carbonyl)-3-phenylpiperidine-4-carbonyl]piperidin-4-yl}methyl)pyrimidin-4(3H)-one (Example 34)

Using General Procedure 11 starting from EXAMPLE 33 and (6-methyl-3-pyridyl)boronic acid as reagents. EXAMPLE 34 was obtained. HRMS calculated for C₄₀H₄₀FN₇O₅: 717.3075; found 718.3149 ((M+H)⁺ form).

### 5-amino-6-(4-fluorophenoxy)-3-({4-hydroxy-1-[(3R,4R)-3-phenyl-1-(5-phenylpyridine-3-carbonyl)piperidine-4-carbonyl]piperidin-4-yl}methyl)pyrimidin-4(3H)-one (Example 35)

Using General Procedure 11 starting from EXAMPLE 33 and phenylboronic acid as reagents. EXAMPLE 35 was obtained. HRMS calculated for C₄₀H₃₉FN₆O₅: 702.2966: found 703.3035 ((M+H)⁺ form).

### 5-amino-3-({1-(3R,4R)-1-benzoyl-3-phenylpiperidine-4-carbonyl]-4-hydroxypiperidin-4-yl}methyl)-6-(4-fluorophenoxy)pyrimidin-4(3H)-one (Example 36)

Using General Procedure 6 starting from EXAMPLE 21 and benzoic acid as reagents, EXAMPLE 36 was obtained. HRMS calculated for C₃₅H₃₆FN₅O₅: 625.27: found 626.2775 ((M+H)⁺ form).

### 5-amino-6-(4-fluorophenoxy)-3-({4-hydroxy-1-[(3R,4R)-1-(1-methyl-1H-indole-2-carbonyl)-3-phenylpiperidine-4-carbonyl]piperidin-4-yl}methyl)pyrimidin-4(3H)-one (Example 37)

Using General Procedure 6 starting from EXAMPLE 21 and 1-methylindole-2-carboxylic acid as reagents, EXAMPLE 37 was obtained. HRMS calculated for C₃₈H₃₉FN₆O₅: 678.2966; found 679.3032 ((M+H)⁺ form).

### 5-amino-6-(4-fluorophenoxy)-3-[4-hydroxy-1-[(3R,4R)-1-(1-methyl-S-phenyl-pyrrole-2-carbonyl)-3-phenyl-piperidine-4-carbonyl]-4-piperidyl]methyl]pyrimidin-4-one (Example 38)

Using General Procedure 6 starting from EXAMPLE 21 and 1-methyl-5-phenyl-pyrrole-2-carboxylic acid as reagents, EXAMPLE 38 was obtained. HRMS calculated for C₄₀H₄₁FN₆O₅: 704.3123; found 705.3197 ((M+H)⁺ form).

### 5-amino-3-({1-[(3R,4R)-1-(3-fluoro-5-iodothiophene-2-carbonyl)-3-phenylpiperidine-4-carbonyl]-4-hydroxypiperidin-4-yl}methyl)-6-(4-fluorophenoxy)pyrimidin-4(3H)-one (Example 39)

Using General Procedure 6 starting from EXAMPLE 21 and Preparation R6a as reagents, EXAMPLE 39 was obtained. HRMS calculated for C₃₃H₃₂F₂IN₅O₅S: 775.1137; found 776.1198 ((M+H)⁺ form).

### 5-amino-3-[(1-{(3R,4R)-1-[3-fluoro-5-(6-methylpyridin-3-yl)thiophene-2-carbonyl]-3-phenylpiperidine-4-carbonyl)-4-hydroxypiperidin-4-yl)methyl-6-(4-fluorophenoxy)pyrimidin-4(3H)-one (Example 40)

Using General Procedure 11 starting from EXAMPLE 39 and (6-methyl-3-pyridyl)boronic acid as reagents. EXAMPLE 40 was obtained. HRMS calculated for C₃₉H₃₈F₂N₆O₅S: 740.2592; found 741.2675 ((M+H)⁺ form).

### 5-amino-3-(((4S)-1-[(3R,4R)-1-(2-bromo-4-methyl-1,3-thiazole-5-carbonyl)-3-phenylpiperidine-4-carbonyl]-3,3-difluoro-4-hydroxypiperidin-4-yl}methyl)-6-(4-fluorophenoxy)pyrimidin-4(3H)-one (Example 41)

Using General Procedure 6 starting from EXAMPLE 11 and 2-bromo-4-methyl-thiazole-5-carboxylic acid as reagents, EXAMPLE 41 was obtained. HRMS calculated for C₃₃H₃₂BrF₃N₆O₅S: 760.129; found 761,139 ((M+H)⁺ form).

### 5-amino-6-(4-fluorophenoxy)-3-({4-hydroxy-1-{(3R,4R)-1-{4-methyl-2-[6-(trifluromethyl)pyridin-3-yl]-1,3-thiazole-5carbonyl)-3-phenylpiperidine-4-carbonyl]piperidin-4-yl}methyl)pyrimidin-4(3H)-one (Example 42)

Using General Procedure 11 starting from EXAMPLE 29 and 16-(trifluoromethyl)-3-pyridyl]boronic acid as reagents. EXAMPLE 42 was obtained. HRMS calculated for C₃₉H₃₇F₄N₇O₅S: 791.2513: found 792,258 ((M+H)⁺ form).

### 5-amino-3-({1-[(3R,4R)-1-{2-[16-(dimethylamino)pyridin-3-yl-4-methyl-1,3-thiazole-5-carbonyl}-3-phenylpiperidine-4-carbonyl]-4-hydroxypiperidin-4-yl]}methyl)-6-(4-fluorophenoxy)pyrimidin-4(3H)-one (Example 43)

Using General Procedure 11 starting from EXAMPLE 29 and [6-(dimethylamino)-3-pyridyl]boronic acid as reagents. EXAMPLE 43 was obtained. HRMS calculated for C₄₀H₄₃FN₈O₅S: 766.3061: found 767.3131 ((M+H)⁺ form).

### 5-amino-3-{(4S)-3,3-difluoro-1-((3R,4R)-1-[2-(4-fluorophenyl)-4-methyl-1,3-thiazole-5-carbonyl]-3-phenylpiperidine-4-carbonyl}-4-hydroxypiperidin-4-yl]methyl}-6-(4-fluorophenoxy)pyrimidin-4(3H)-one (Example 44)

Using General Procedure 11 starting from EXAMPLE 41 and (4-fluorophenyl)boronic acid as reagents, EXAMPLE 44 was obtained. HRMS calculated for C₃₉H₃₆F₄N₆O₅S: 776.2404; found 777.2481 ((M+H)⁺ form).

### 5-amino-3-{1(4S)-3,3-difluoro-4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl]methyl}-6-(4-fluorophenoxy)pyrimidin-4(3H)-one (Example 45)

Using General Procedure 6 starting from EXAMPLE 11 and 4-methyl-2-(6-methyl-3-pyridyl)thiazole-5-carboxylic acid as reagents, EXAMPLE 45 was obtained. HRMS calculated for C₃₉H₃₈F₃N₇O₅S: 773.2607: found 774.2668 ((M+H)⁺ form).

### 5-amino-3-[(4-hydroxy-1-((3R,4R)-1-[4-methyl-2-(6-methylpyridin-3-yl)-1,3-thiazole-5-carbonyl]-3-phenylpiperidine-4-carbonyl)piperidin-4-yl)methyl)-6-phenoxypyrimidin-4(3H)-one (Example 46)

Using General Procedure 6 starting from EXAMPLE 20 and 4-methyl-2-(6-methyl-3-pyridyl)thiazole-5-carboxylic acid as reagents, EXAMPLE 46 was obtained. HRMS calculated for C₃₉H₄₁N₇O₅S: 719.289: found 720.2948 ((M+H)⁺ form).

### 5-amino-6-(4-fluorophenoxy)-3-[(4-hydroxy-1-{(3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one (Example 47)

Using General Procedure 6 starting from EXAMPLE 21 and 4-methyl-2-(6-methyl-3-pyridyl)thiazole-5-carboxylic acid as reagents. EXAMPLE 47 was obtained. HRMS calculated for C₃₉H₄₀FN₇O₅S: 737,2795; found 738.2863 ((M+H)⁺ form).

### 5-amino-6-(4-fluorophenoxy)-3-[(1-((3R,4R)-1-[2-(4-fluorophenyl)-4-methyl-1,3-thiazole-5-carbonyl]-3-phenylpiperidine-4-carbonyl}-4-hydroxypiperidin-4-yl)methyl]pyrimidin-4(3H)-one (Example 48)

Using General Procedure 11 starting from EXAMPLE 29 and (4-fluorophenyl)boronic acid as reagents, EXAMPLE 48 was obtained. HRMS calculated for C₃₉H₃₈F₂N₆O₅S: 740.2592; found 741.2661 ((M+H)⁺ form).

### 5-amino-6-(4-fluorophenoxy)-3-j 14-hydroxy-1-[(3R,4R)-1-(2-methylthiazole-5-carbonyl)-3-phenyl-piperidine-4-carbonyl]-4-piperidyl]methyl]pyrimidin-4-one (Example 49)

Using General Procedure 6 starting from EXAMPLE 21 and 2-methylthiazole-5-carboxylic acid as reagents. EXAMPLE 49 was obtained. HRMS calculated for C₃₃H₃₅FN₆O₅S: 646.2374: found 647.2452 ((M+H)⁺ form),

### 5-amino-3-[[1-[(3R,4R)-1-[(2-bromothiazol-5-yl)methyl]-3-phenyl-piperidine-4-carbonyl]-4-hydroxy-4-piperidyl]methyl]-6-(4-fluorophenoxy)pyrimidin-4-one (Example 50)

Using General Procedure 9 starting from EXAMPLE 21 and 2-bromo-5-(bromomethyl)thiazole as reagents, EXAMPLE 50 was obtained. HRMS calculated for C₃₂H₃₄BrFN₆O₄S: 696.153: found 697.1602 ((M+H)⁺ form).

### 5-amino-6-(4-fluorophenoxy)-3-({4-hydroxy-1-[(3R,4R)-1-{{2-(6-methylpyridin-3 yl)-1,3-thiazol-5-yl]methyl}-3-phenylpiperidine-4-carbonyl]piperidin-4-yl}methyl)pyrimidin-4(3H)-one (Example 51)

Using General Procedure 10 starting from EXAMPLE 21 and 2-(6-methyl-3-pyridyl)thiazole-5-carbaldehyde as reagents, EXAMPLE 51 was obtained. HRMS calculated for C₃₈H₄₀FN₇O₄S: 709.2947; found 710.2918 ((M+H)⁺ form).

### 5-amino-6-(4-fluorophenoxy)-3-[(1-((3R,4R)-1-[(2-fluorophenyl)methyl]-3-phenylpiperidine-4-carbonyl}-4-hydroxypiperidin-4-yl)methyl]pyrimidin-4(3H)-one (Example 52)

Using General Procedure 10 starting from EXAMPLE 21 and 2-fluorobenzaldehyde as reagents, EXAMPLE 52 was obtained. HRMS calculated for C₃₅H₃₇F₂N₅O₄: 629,2814; found 630.2878 ((M+H)⁺ form).

### 5-amino-3-({(4S)-3,3-difluoro-4-hydroxy-1-[(3R,4R)-1-{12-(6-methylpyridin-3-yl)-1,3-thiazol-5-yl]methyl)-3-phenylpiperidine-4-carbonyl[piperidin-4-yl]methyl)-6-(4-fluorophenoxy)pyrimidin-4(3H)-one (Example 53)

Using General Procedure 10 starting from EXAMPLE 11 and 2-(6-methyl-3-pyridyl)thiazole-5-carbaldehyde as reagents, EXAMPLE 53 was obtained, H₁RMS calculated for C₃₈H₃₈F₃N₇O₄S: 745.2658; found 746.2726 ((M+H)⁺ form).

### 5-amino-3-{[(4S)-3,3-difluoro-1-((3R,4R)-1-[(2-fluorophenyl)methyl]-3-phenylpiperidine-4-carbonyl}-4-hydroxypiperidin-4-yl}methyl)-6-(4-fluorophenoxy)pyrimidin-4(3H)-one (Example 54)

Using General Procedure 10 starting from EXAMPLE 11 and 2-fluorobenzaldehyde as reagents. EXAMPLE 54 was obtained. HRMS calculated for C₃₅H₃₅F₄N₅O₄: 665.2625; found 666.2686 ((M+H)⁺ form).

### 5-amino-3-({1-[(3R,4R)-1-(3-ethoxybenzoyl)-3-phenylpiperidine-4-carbonyl]-4-hydroxypiperidin-4-yl)methyl)-6-(4-fluorophenoxy)pyrimidin-4(3H)-one (Example 55)

Using General Procedure 6 starting from EXAMPLE 21 and 3-ethoxybenzoic acid as reagents, EXAMPLE 55 was obtained. HRMS calculated for C₃₇H₄₀FN₅O₆: 669.2963; found 670.3033 ((M+H)⁺ form).

### 5-amino-6-(4-fluorophenoxy)-3-[[4-hydroxy-1-(3R,4R)-1-[3-(4-methylpiperazin-1-yl)benzoyl]-3-phenyl-piperidine-4-carbonyl]-4-piperidyl]methyl]pyrimidin-4-one (Example 56)

Using General Procedure 6 starting from EXAMPLE 21 and 3-(4-methylpiperazin-1-yl)benzoic acid as reagents, EXAMPLE 56 was obtained. HRMS calculated for C₄₀H₄₆FN₇O₅: 723.3544: found 724.3609 ((M+H)⁺ form).

### 6-[(3R,4R)-4-(4-t[5-amino-4-(4-fluorophenoxy)-6-oxopyrimidin-[(6N)-yl]methyl}-4-hydroxypiperidine-1-carbonyl)-3-phenylpiperidine-1-carbonyl]quinazolin-4(3n)-one (Example 57)

Using General Procedure 6 starting from EXAMPLE 21 and 4-oxo-311-quinazoline-6-carboxylic acid as reagents, EXAMPLE 57 was obtained. HRMS calculated for C₃₇H₃₆FN₇O₆: 693.2711; found 694.2768 ((M+H)⁺ form).
5-[(3R,4R)-4-[4-[[5-amino-4-(4-fluorophenoxy)-6-oxo-pyrimidin-1-yl]methyl]-4-hydroxy-piperidine-1-carbonyl]-3-phenyl-piperidine-1-carbonyl]indoline-2,3-dione (Example 58)

Using General Procedure 6 starting from EXAMPLE 21 and 2,3-dioxoindoline-5-carboxylic acid as reagents, EXAMPLE 58 was obtained. HRMS calculated for C₃₇H₃₅FN₆O₇: 694.2551; found 695.2622 ((M+H)⁺ form).

### 5-amino-3-[[1-[(3R,4R)-1-[(5-bromo-2-thienyl)sulfonyl]-3-phenyl-piperidine-4-carbonyl-4-hydroxy-4-piperidyl]methyl]-6-(4-fluorophenoxy)pyrimidin-4-one (Example 59)

Using General Procedure 9 starting from EXAMPLE 21 and 5-bromothiophene-2-sulfonyl chloride as reagents, EXAMPLE 59 was obtained. HRMS calculated for C₃₂H₃₃BrFN₅O₆S₂: 745,1039; found 746.1123 ((M+H)⁺ form)

### (3R,4R)-4-[4-[[5-amino-4-(4-fluorophenoxy)-6-oxo-pyrimidin-1-yl[methyl]-4-hydroxy-piperidine-1-carbonyl]-N,3-diphenyl-piperidine-1-carbothioamide (Example 60)

Using General Procedure 7 starting from EXAMPLE 21 and isothiocyanatobenzene as reagents, Example 60 was obtained. HRMS calculated for C₃₅H₃₇FN₆O₄S: 656.2581; found 657.2656 ((M+H)⁺ form)

### 5-amine-3-({1-[(3R,4R)-1-(2-benzyl-4-methyl-1,3-thiazole-5-carbonyl)-3-phenylpiperidine-4-carbonyl]-4-hydroxypiperidin-4-yl)methyl)-6-(4-fluorophenoxy)pyrimidin-4(3H)-one (Example 61)

Using General Procedure 6 starting from EXAMPLE 21 and 2-benzyl-4-methyl-thiazole-5-carboxylic acid as reagents, EXAMPLE 61 was obtained. HRMS calculated for C₄₀H₄₁FN₆O₅S: 736.2843; found 737.2903 ((M+H)⁺ form).

### 5-amino-6-(4-fluorophenoxy)-3-[(4-hydroxy-1-{(3R,4R)-3-phenyl-1-[(pyridin-3-yl)methyl]piperidine-4-carbonyl)piperidin-4-yl)methyl]pyrimidin-4(3H)-one (Example 62)

Using General Procedure 9 starting from EXAMPLE 21 and 3-(chloromethyl)pyridine hydrochloride as reagents, EXAMPLE 62 was obtained. HRMS calculated for C₃₄H₃FN₆O₄: 612.286; found 613.2922 ((M+H)⁺ form).

### 5-amino-6-(4-fluorophenoxy)-3-[[4-hydroxy-1-[(3R,4R)-3-phenyl-1-(pyrazin-2-yl)methyl)piperidine-4-carbonyl]-4-piperidyl]methyl]pyrimidin-4-one (Example 63)

Using General Procedure 9 starting from EXAMPLE 21 and 2-(chloromethyl)pyridine hydrochloride as reagents, EXAMPLE 63 was obtained. HRMS calculated for C₃₃H₃₇FN₆O₄: 613.2813; found 614.2890 ((M+H)⁺ form)

### 5-amino-6-(4-fluorophenoxy)-3-[(4-hydroxy-1-{(3R,4R)-1-[4-methyl-2-(morpholin-4-yl)-1,3-thiazole-5-carbonyl]-3-phenylpiperidine-4-carbonyl)piperidin-4-yl)methyl]pyrimidin-4(3H)-one (Example 64)

Using General Procedure 6 starting from EXAMPLE 21 and 4-methyl-2-morpholino-thiazole-5-carboxylic acid as reagents, EXAMPLE 64 was obtained. HRMS calculated for C₃₇H₄₂FN₇O₆S: 731.2902; found 732.2964 ((M+H)⁺ form).

### 5-amino-6-(4-fluorophenoxy)-3-[(4-hydroxy-1-{(3R,4R)-1-[4-methyl-2-(pyrrolidin-1-yl)-1,3-thiazole-5-carbonyl]-3-phenylpiperidine-4-carbonyl]piperidin-4-yl)methyl]pyrimidin-4(3H)-one (Example 65)

Using General Procedure 6 starting from EXAMPLE 21 and 4-methyl-2-pyrrolidin-1-yl-thiazole-5-carboxylic acid as reagents, EXAMPLE 65 was obtained. HRMS calculated for C₃₇H₄₂FN₇O₅S: 715.2952: found 716.3019 ((M+H)⁺ form).

### 5-amino-6-(4-fluorophenoxy)-3-({4-hydroxy-1-[(3R,4R)-1-(4-methyl-2-phenyl-1,3-thiazole-5-carbonyl)-3-phenylpiperidine-4-carbonyl]piperidin-4-yl}methyl)pyrimidin-4(3H)-one (Example 66)

Using General Procedure 6 starting from EXAMPLE 21 and 4-methyl-2-phenyl-thiazole-5-carboxylic acid as reagents, EXAMPLE 66 was obtained. HRMS calculated for C₃₉H₃₉FN₆O₅S: 722.2687; found 723.2770 ((M+H)⁻ form).

### 5-amino-3-[[1-[(3R,4R)-1-(5-bromo-2,3-dihydrothieno[3,4-b][1,4]dioxine-7-carbonyl)-3-phenyl-piperidine-4-carbonyl]-4-hydroxy-4-piperidyl]methyl-6-(4-fluorophenoxy)pyrimidin-4-one (Example 67)

Using General Procedure 6 starting from EXAMPLE 21 and 5-bromo-2,3-dihydrothieno[3,4-b][1,4]dioxine-7-carboxylic acid as reagents, EXAMPLE 67 was obtained. HRMS calculated for C₃₅H₃₅BrFN₅O₇S: 767.1425: found 768,1486 ((M+H)⁺ form).

### 5-amino-6-(4-fluorophenoxy)-3-[(4-hydroxy-1-{(3R,4R)-1-[7-(6-methylpyridin-3-yl)-2,3-dihydrothieno[3,4-b][1,4]dioxine-5-carbonyl]-3-phenylpiperidine-4-carbonyl]piperidin-4-yl)methyl]pyrimidin-4(3H)-one (Example 68)

Using General Procedure 11 starting from EXAMPLE 67 and (6-methyl-3-pyridyl)boronic acid as reagents, EXAMPLE 68 was obtained. HRMS calculated for C₄₁H₄₁FN₆O₇S: 780.2742; found 781.2803 ((M+H)⁺ form).

### 5-amino-3-[[1-[(3R,4R)-1-(3-bromophenyl)sulfonyl-3-phenyl-piperidine-4-carbonyl]-4-hydroxy-4-piperidyl]methyl]-6-(4-fluorophenoxy)pyrimidin-4-one (Example 69)

Using General Procedure 9 starting from EXAMPLE 21 and 3-bromobenzenesulfonyl chloride as reagents, EXAMPLE 69 was obtained. HRMS calculated for C₃₄H₃₅BrFN₅O₆S: 739.1475: found 740.1540 ((M+H)⁺ form)

### ethyl 2-[(3R,4R)-4-[4-[[5-amino-4-(4-fluorophenoxy)-6-oxo-pyrimidin-1-yl]methyl-4-hydroxy-piperidine-1-carbonyl]-3-phenyl-1-piperidyl]-2-phenyl-acetate (Example 70)

Using General Procedure 9 starting from EXAMPLE 21 and ethyl 2-bromo-2-phenyl-acetate as reagents. EXAMPLE 70 was obtained. HRMS calculated for C₃₈H₄₂FN₅O₆: 683.3119: found 684.3224 and 684.3192 ((M+H)⁻ form).

### 5-amino-3-[[(4S)-3,3-difluoro-4-hydroxy-1-[(3R,4R)-3-phenyl-1-(3-phenylcyclobutanecarbonyl)piperidine-4-carbonyl]-4-piperidyl]methyl]-6-(4-fluorophenoxy)pyrimidin-4-one, diastereoisomer 1 (Example 71)

### and

### 5-amino-3-[[(4S)-3,3-difluoro-4-hydroxy-1-[(3R,4R)-3-phenyl-1-(3-phenylcyclobutanecarbonyl)piperidine-4-carbonyl]-4-piperidyl]methyl]-6-(4-fluorophenoxy)pyrimidin-4-one, diastereoisomer 2 (Example 72)

Using General Procedure 6 starting from EXAMPLE 11 and 3-phenylcyclobutanecarboxylic acid as reagents, the enantiomers were separated by chiral chromatography to give EXAMPLE 71 and EXAMPLE 72.

EXAMPLE 71: HRMS calculated for C₃₉H₄₀F₃N₅O₅: 715.2982: found 716.3053 ((M+H)⁺ form).

EXAMPLE 72: HRMS calculated for C₃₉H₄₀F₃N₅O₅: 715.2982: found 716,3044 ((M+H)⁺ form).

### 5-amino-6-(4-fluorophenoxy)-3-[[4-hydroxy-1-[(3R,4R)-1-(2-hydroxy-2-methyl-propyl)-3-phenyl-piperidine-4-carbonyl]-4-piperidyl]methyl]pyrimidin-4-one (Example 73)

Using General Procedure 9 starting from EXAMPLE 21 and 2,2-dimethyloxirane as reagents, EXAMPLE 73 was obtained. HRMS calculated for C₃₂H₄₀FN₅O₅: 593,3013; found 594.3078 (M+H)⁺ form).

### 2-[(3R,4R)-4-[4-[[5-amino-4-(4-fluorophenoxy)-6-oxo-pyrimidin-1-yl]methyl]-4-hydroxy-piperidine-1-carbonyl-3-phenyl-1-piperidyl]-N-methyl-N-phenyl-acetamide (Example 74)

Using General Procedure 9 starting from EXAMPLE 21 and 2-bromo-N-methyl-N-phenyl-acetamide as reagents, EXAMPLE 74 was obtained. HRMS calculated for C₃₇H₄₁FN₆O₅: 668.3123; found 669.3199 ((M+H)⁺ form).

### 5-amino-3-[[1-[(3R,4R)-1-(2,2-difluoro-2-phenyl-acetyl)-3-phenyl-piperidine-4-carbonyl]-4-hydroxy-4-piperidyl]methyl]-6-(4-fluorophenoxy)pyrimidin-4-one (Example 75)

Using General Procedure 6 starting from EXAMPLE 21 and 2,2-difluoro-2-phenyl-acetic acid as reagents, EXAMPLE 75 was obtained. HRMS calculated for C₃₆H₃₆F₃N₅O₅: 675.2668; found 676.274 ((M+H)⁺ form).

### (3R,4R).4-[4-[[5-amino-4-(4-fluorophenoxy)-6-oxo-pyrimidin-1-yl]methyl]-4-hydroxy-piperidine-1-carbonyl]-N,3-diphenyl-piperidine-1-carboxamide (Example 76)

Using General Procedure 7 starting from EXAMPLE 21 and isocyanatobenzene as reagents. EXAMPLE 76 was obtained. HRMS calculated for C₃₅H₃₇FN₆O₅: 640.2809; found 641.2878 ((M+H)⁺ form).

### (3R,4R)-4-[(4S)-4-[[5-amino-4-(4-fluorophenoxy)-6-oxo-pyrimidin-1-yl]methyl]-3,3-difluoro-4-hydroxy-piperidine-1-carbonyl-N,3-diphenyl-piperidine-1-carboxamide (Example 77)

Using General Procedure 7 starting from EXAMPLE 11 and isocyanatobenzene as reagents, EXAMPLE 77 was obtained. HRMS calculated for C₃₅H₃₅F₃N₆O₅: 676.2621: found 677.269 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-N-methyl-N,3-diphenyl-piperidine-1-carboxamide (Example 78)

EXAMPLE 21 (150 mg, 0.2786 mmol), N-methyl-N-phenyl-carbamoyl chloride (1.3 eq., 0.373 mmol). N,N-diethylethanamine (2.0 eq., 0.5752 mmol) were dissolved in DCM (5 mL). The mixture was stirred at r.t. for 1 hour, then 5 ml isopropylalcohol was added, then it was purified by preparative LC (on C-18 Gemini-NX 5 μm column, 5 mM aqueous NH₄HCO₃-MeCN, gradient). Solvent was evaporated under reduced pressure to give EXAMPLE 78. HRMS calculated for C₃₆H₃₉FN₆O₅: 654.2966; found 655.3051 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-N-benzyl-3-phenyl-piperidine-1-carboxamide (Example 79)

Using General Procedure 7 starting from EXAMPLE 21 and isocyanatomethylbenzene as reagents, EXAMPLE 79 was obtained. HRMS calculated for C₃₆H₃₉FN₆O₅: 654.2966; found 655.3025 ((M+H)⁺ form).

### (3R,4R)-4-(4-[[5-amino-4-(4-fluorophenoxy)-6-oxopyrimidin-[(6H)-yl]methyl}-4-hydroxypiperidine-1-carbonyl)-N-(4-methoxyphenyl)-3-phenylpiperidine-1-carboxamide (Example 80)

Using General Procedure 7 starting from EXAMPLE 21 and 1-isocyanato-4-methoxy-benzene as reagents, Example 80 was obtained. HRMS calculated for C₃₆H₃₉FN₆O₆: 670.2915; found 671.299 ((M+H)⁺ form).

### (3R,4R)-4-(4-{15-amino-4-(4-fluorophenoxy)-6-oxopyrimidin-[(6H)-yl]methyl}-4-hydroxypiperidine-1-carbonyl)-3-phenyl-N-[3-(trifluoromethyl)phenyl]piperidine-1-carboxamide (Example 81)

Using General Procedure 7 starting from EXAMPLE 21 and 1-isocyanato-3-(trifluoromethyl)benzene as reagents, EXAMPLE 81 was obtained. HRMS calculated for C₃₆H₃₆F₄N₆O₅: 708.2683: found 709.276 ((M+H)⁺ form).

### (3R,4R)-4-[4-[(5-amino-4-(4-fluorophenoxy)-6-oxo-pyrimidin-1-yl]methyl]-4-hydroxy-piperidine-1-carbonyl-N-(2,4-dimethoxyphenyl)-3-phenyl-piperidine-1-carboxamide (Example 82)

Using General Procedure 7 starting from EXAMPLE 21 and 1-isocyanato-2,4-dimethoxy-benzene as reagents, EXAMPLE 82 was obtained. HRMS calculated for C₃₇H₄₁FN₆O₇: 700.3021: found 701.3091 ((M+H)⁺ form).

### (3R,4R)-4-(4-{[5-amino-4-(4-fluorophenoxy)-6-oxopyrimidin-[(6H)-yl}methyl}-4-hydroxypiperidine-1-carbonyl)-N-(3-bromophenyl)-3-phenylpiperidine-1-carboxamide (Example 83)

Using General Procedure 7 starting from EXAMPLE 21 and 1-bromo-3-isocyanato-benzene as reagents, EXAMPLE 83 was obtained. HRMS calculated for C₃₅H₃₆BrFN₆O₅: 718.1915: found 719.1973 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-N-(4-fluorophenyl)-3-phenyl-piperidine-1-carboxamide (Example 84)

Using General Procedure 7 starting from EXAMPLE 21 and 1-fluoro-4-isocyanato-ben/ene as reagents, EXAMPLE 84 was obtained. HRMS calculated for C₃₅H₃₆F₂N₆O₅: 658.2715: found 659.2782 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-N-[3-(6-methyl-3-pyridyl)phenyl]-3-phenyl-piperidine-1-carboxamide (Example 85)

Using General Procedure 11 starting from EXAMPLE 83 and (6-methyl-3-pyridyl)boronic acid as reagents, EXAMPLE 85 was obtained. HRMS calculated for C₄₁H₄₂FN₂O₅: 731.3231; found 732.3295 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-3-phenyl-N-thiazol-2-yl-piperidine-1-carboxamide (Example 86)

Using General Procedure 8 starting from EXAMPLE 21 and thiazol-2-amine as reagents, EXAMPLE 86 was obtained. HRMS calculated for C₃₂H₃₄FN₇O₅S: 647.2326; found 648.2396 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-N-(1,3-benzothiazol-2-yl)-3-phenyl-piperidine-1-carboxamide (Example 87)

Using General Procedure 8 starting from EXAMPLE 21 and 1,3-benzothiazol-2-amine as reagents, EXAMPLE 87 was obtained. HRMS calculated for C₃₆H₃₆FN₇O₅S: 697.2483; found 698.2558 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-3-phenyl-N-[4-(trifluoromethyl)thiazol-2-yl]piperidine-1-carboxamide (Example 88)

Using General Procedure 8 starting from EXAMPLE 21 and 4-(trifluoromethyl)thiazol-2-amine as reagents, EXAMPLE 88 was obtained. HRMS calculated for C₃₃H₃₃F₄N₇O₅S: 715.22; found 716.2273 ((M+H)⁺ form).

### (3R,4R)-4-[4-[[5-amino-4-(4-fluorophenoxy)-6-oxo-pyrimidin-1-yl]methyl-4-hydroxy-piperidine-1-carbonyl-N-(2-fluorophenyl)-3-phenyl-piperidine-1-carboxamide (Example 89)

Using General Procedure 7 starting from EXAMPLE 21 and 1-fluoro-2-isocyanato-benzene as reagents. EXAMPLE 89 was obtained. HRMS calculated for C₃₅H₃₆F₂N₆O₅: 658.2715: found 659.2779 ((M+H)⁺ form).

### (3R,4R)-4-[4-1l5-amino-4-(4-fluorophenoxy)-6-oxo-pyrimidin-1-yl]methyl-4-hydroxy-piperidine-1-carbonyl]-3-phenyl-N-(2,2,2-trifluoroethyl)piperidine-1-carboxamide (Example 90)

Using General Procedure 8 starting from EXAMPLE 21 and 2,2,2-trifluoroethanamine as reagents. EXAMPLE 90 was obtained. HRMS calculated for C₃₁H₃₄F₄N₅O₅: 646.2527; found 647.2617 ((M+H)⁺ form).

### 5-amino-6-(4-fluorophenoxy)-3-[[4-hydroxy-1-[(3R,4R)-1-(2-methylpyridine-4-carbonyl)-3-phenyl-piperidine-4-carbonyl]4-piperidyl]methyl]pyrimidin-4-one (Example 91)

Using General Procedure 6 starting from EXAMPLE 21 and 2-methylpyridine-4-carboxylic acid as reagents, EXAMPLE 91 was obtained. HRMS calculated for C₃₅H₃₇FN₆O₅: 6401809; found 641.28822 (M+H)⁺ form).

### 5-amino-6-(4-fluorophenoxy)-3-({4-hydroxy-1-[(3R,4R)-1-(5-methylpyridine-3-carbonyl)-3-phenylpiperidine-4-carbonyl]piperidin-4-yl}methyl)pyrimidin-4(3H)-one (Example 92)

Using General Procedure 6 starting from EXAMPLE 21 and 5-methylpyridine-3-carboxylic acid as reagents, EXAMPLE 92 was obtained. HRMS calculated for C₃₅H₃₇FN₆O₅: 640.2809; found 641.28802 ((M+H)⁺ form).

### 5-amino-6-(4-fluorophenoxy)-3-[[4-hydroxy-1-[(3R,4R)-1-(6-methylpyridine-2-carbonyl)-3-phenyl-piperidine-4-carbonyl-4-piperidyl]methyl]pyrimidin-4-one (Example 93)

Using General Procedure 6 starting from EXAMPLE 21 and 6-methylpyridine-2-carboxylic acid as reagents, EXAMPLE 93 was obtained. HRMS calculated for C₃₅H₃₇FN₆O₅: 640.2809; found 641.2876 ((M+H)⁺ form),

### 5-amino-6-(4-fluorophenoxy)-3-[[4-hydroxy-1-[(3R,4R)-1-(4-methylpyridine-2-carbonyl)-3-phenyl-piperidine-4-carbonyl]-4-piperidyl]methyl]pyrimidin-4-one (Example 94)

Using General Procedure 6 starting from EXAMPLE 21 and 4-methylpyridine-2-carboxylic acid as reagents, EXAMPLE 94 was obtained. HRMS calculated for C₃₅H₃₇FN₆O₅: 640,2809; found 641.2868 ((M+H)⁺ form).

### 5-amino-3-({1-[(3R,4R)-1-(2-bromo-1-methyl-11H-imidazole-5-carbonyl)-3-phenylpiperidine-4-carbonyl]-4-hydroxypiperidin-4-yl}methyl)-6-(4-fluorophenoxy)pyrimidin-4(3H)-one (Example 95)

Using General Procedure 6 starting from EXAMPLE 21 and 2-bromo-1-methyl-1H-imidazole-5-carboxylic acid as reagents, EXAMPLE 95 was obtained, HRMS calculated for C₃₃H₃₅BrFN₇O₅: 707.1867; found 708.1924 ((M+H)⁺ form).

### 5-amino-6-(4-fluorophenoxy)-3-[(4-hydroxy-1-{(3R,4R)-1-[1-methyl-2-(6-methylpyridin-3-yl)-1H-imidazole-5-carbonyl]-3-phenylpiperidine-4-carbonyl}piperidin-4-yl)methyl]pyrimidin-4(3H)-one (Example 96)

Using General Procedure 11 starting from EXAMPLE 95 and (6-methyl-3-pyridyl)boronic acid as reagents, EXAMPLE 96 was obtained. HRMS calculated for C₃₉H_{4I}FN₈O₅: 720.3184; found 721.3253 ((M+H)⁺ form)

### 5-amino-3-({1-[(3R,4R)-1-(2,6-dimethylpyridine-4-carbonyl)-3-phenylpiperidine-4-carbonyl]-4-hydroxypiperidin-4-yl}methyl)-6-(4-fluorophenoxy)pyrimidin-4(3H)-one (Example 97)

Using General Procedure 6 starting from EXAMPLE 21 and 2,6-dimethylpyridine-4-carboxylic acid as reagents, EXAMPLE 97 was obtained. HRMS calculated for C₃₆H₃₉FN₆O₅: 654,29%6; found 654.2966 ((M+H)⁺ form).

### 5-amino-3-(l1-[(3R,4R)-1-(3-bromo-5-fluorobenzoyl)-3-phenylpiperidine-4-carbonyl]-4-hydroxypiperidin-4-yl}methyl)-6-(4-fluorophenoxy)pyrimidin-4(3H)-one (Example 98)

Using General Procedure 6 starting from EXAMPLE 21 and 3-bromo-5-fluoro-benzoic acid as reagents, EXAMPLE 98 was obtained. HRMS calculated for C₃H₃₄BrF₂N₅O₅: 721.1711; found 722.1787 ((M+H)⁺ form).

### 5-amino-3-[[1-((3R,4R)-1-(3-fluoro-5-iodo-thiophene-2-carbonyl)-3-phenyl-piperidine-4-carbonyl]-4-hydroxy-4-piperidyl]methyl]-6-[3-(trifluoromethyl)phenoxy]pyrimidin-4-one (Example 99)

Using General Procedure 6 starting from EXAMPLE 18 and Preparation R6a as reagents, EXAMPLE 99 was obtained. HRMS calculated for C₃₄H₃₂F₄IN₅O₅S: 825.1105; found 826.12 ((M+H)⁺ form).

### 5-amino-3-[[1-[(3R,4R)-1-(2-bromo-4-methyl-thiazole-5-carbonyl)-3-phenyl-piperidine-4-carbonyl]-4-hydroxy-4-piperidyl]methyl]-6-[3-(trifluoromethyl)phenoxy]pyrimidin-4-one (Example 100)

Using General Procedure 6 starting from EXAMPLE 18 and 2-bromo-4-methyl-thiazole-5-carboxylic acid as reagents, EXAMPLE 100 was obtained. HRMS calculated for C₃₄H₃₄BrF₃N₆O₅S: 774.1447; found 775.1515 ((M+H)⁺ form),

### 5-amino-3-[[1-[(3R,4R)-1-[3-fluoro-5-(46-methyl-3-pyridyl)thiophene-2-carbonyl]-3-phenyl-piperidine-4-carbonyl]-4-hydroxy-4-piperidyl]methyl]-6-[3-(trifluoromethyl)phenoxy]pyrimidin-4-one (Example 101)

Using General Procedure 11 starting from EXAMPLE 99 and (6-methyl-3-pyridyl)boronic acid as reagents. EXAMPLE 101 was obtained. HRMS calculated for C₄₀H₃₈F₄N₆O₅S: 790.256: found 791.2615 ((M+H)⁺ form)

### 5-amino-6-(4-fluorophenoxy)-3-({4-hydroxy-1-[(3R,4R)-1-(isoquinoline-5-carbonyl)-3-phenylpiperidine-4-carbonyl]piperidin-4-yl}methyl)pyrimidin-4(3H)-one (Example 102)

Using General Procedure 6 starting from EXAMPLE 21 and isoquinoline-5-carboxylic acid as reagents, EXAMPLE 102 was obtained. HRMS calculated for C₃₈H₃₇FN₆O₅: 676.2809; found 677.28794 ((M+H)⁺ form).

### 5-amino-3-({(4S)-3,3-difluoro-4-hydroxy-1-[(3R,4R)-1-{4-methyl-2-[6-(trifluoromethyl)pyridin-3-yl]-1,3-thiazole-5-carbonyl])-3-phenylpiperidine-4-carbonyl]piperidin-4-yl]methyl)-6-(4-fluorophenoxy)pyrimidin-4(3)-one (Example 103)

Using General Procedure 11 starting from EXAMPLE 41 and 16-(trifluoromethyl)-3-pyridyl}boronic acid as reagents, EXAMPLE 103 was obtained. HRMS calculated for C₃₉H₃₅F₆N₇O₅S: 827,2325; found 828.2406 ((M+H)⁺ form).

### 5-amino-3-[[(4S)-3,3-difluoro-1-[(3R,4R)-1-(3-fluoro-5-iodo-thiophene-2-carbonyl)-3-phenyl-piperidine-4-carbonyl]-4-hydroxy-4-piperidyl]methyl]-6-(4-fluorophenoxy)pyrimidin-4-one (Example 104)

Using General Procedure 6 starting from EXAMPLE 11 and Preparation R6a as reagents, EXAMPLE 104 was obtained. HRMS calculated for C₃₅H₃₀F₄IN₅O₅S: 811,0948; found 812.1033 ((M+H)⁺ form).

### 5-amino-3-([(4S)-3,3-difluoro-1-((3R,4R)-1-[3-fluoro-5-(6-methylpyridin-3-yl)thiophene-2-carbonyl]-3-phenylpiperidine-4-carbonyl}-4-hydroxypiperidin-4-yl}methyl}-6-(4-fluorophenoxy)pyrimidin-4(3H)-one (Example 105)

Using General Procedure 11 starting from EXAMPLE 104 and (6-methyl-3-pyridyl)boronic acid as reagents, EXAMPLE 105 was obtained. HRMS calculated for C₃₉H₃₆F₄N₆O₅S: 776.24 (4; found 777.2477 ((M+H)⁺ form).

### 5-amino-3-[[I-[(3R,4R)-1-(4-bromo-5-chloro-3-fluoro-thiophene-2-carbonyl)-3-phenyl-piperidine-4-carbonyl]-4-hydroxy-4-piperidyl]methyl]-6-(4-fluorophenoxy)pyrimidin-4-one (Example 106)

Using General Procedure 6 starting from EXAMPLE 21 and Preparation R6b as reagents, EXAMPLE 106 was obtained. HRMS calculated for C₃₃H₃₁BrClF₂N₅O₅S: 761.0886; found 762.09571 ((M+H)⁺ form).

### 5-amino-3-[(1-{(3R,4R)-1-[5-chloro-3-fluoro-4-(6-methylpyridin-3-yl)thiophene-2-carbonyl-3-phenylpiperidine-4-carbonyl)-4-hydroxypiperidin-4-yl)methyl]-6-(4-fluorophenoxy)pyrimidin-4(3H)-one (Example 107)

Using General Procedure 11 starting from EXAMPLE 106 and (6-methyl-3-pyridyl)boronic acid as reagents, EXAMPLE 107 was obtained. HRMS calculated for C₃₉H₃₇ClF₂N₆O₅S: 774.2203; found 775.2268 ((M+H)⁺ form)

### 5-amino-6-(4-fluorophenoxy)-3-[[4-hydroxy-1-((3R,4R)-3-phenyl-1-(3-phenyloxetan-3-yl)piperidine-4-carbonyl]-4-piperidyl]methyl]pyrimidin-4-one (Example 108)

### Step 1: tert-butyl 4-[[5-amino-4-(4-fluorophenoxy)-6-oxo-pyrimidin-yl]methyl]-4-hydroxy-piperidine-1-carboxylate

Using General Procedure 4 starting from Preparation R4h and tert-butyl 1-oxa-6-azaspiro{2.5]octane-6-carboxylate as reagents, tert-butyl 4-[[5-amino-4-(4-fluorophenoxy)-6-oxo-pyrimidin-1-yl]methyl]-4-hydroxy-piperidine-1-carboxylate was obtained. HRMS calculated for C₂₁H₂₇FN₄O₅: 434.1965; found 435.2033 ((M+H)⁺ form).

¹H-NMR (500 MHz, dmso-d6) δ ppm 7.66 (s, 1H), 7.2 (m, 2H), 7.09 (m, 2H), 4.91 (s, 1H), 4.67 (brs., 2H), 3.93 (s, 2H), 3.66/3.05 (brd+br., 4H), 1.4411.33 (td+brd., 4H), 1.39 (s, 9H). ¹³C-NMR (125 MHz, dmso-d6) δ ppm 159.1, 158.8, 154.3, 151, 1468, 139.2, 121.7, 120.2, 116.4, 79, 69.4, 54.4, 39.8, 34.8, 28.6

### Step 2: 5-amino-6-(4-fluorophenoxy)-3-[(4-hydroxy-4-piperidyl)methyl]pyrimidin-4-one hydrochloride

Using General Procedure 5 starting from tert-butyl 4-[[5-amino-4-(4-fluorophenoxy)-6-oxo-pyrimidin-1-yl}methyl]-4-hydroxy-piperidine-1-carboxylate, 5-amino-6-(4-fluorophenoxy)-3-[(4-hydroxy-4-piperidyl)methyl]pyrimidin-4-one hydrochloride was obtained. HRMS calculated for C₁₆H₁₉FN₄O₃: 334.1441: found 335.1508 ((M+H)⁺ form).

¹H-NMR (500 MHz, dmso-d6) δ ppm 9.1/8.88 (m+m, 2H), 8.04 (s, 1H), 7.24 (m, 2H), 7.18 (m, 2H), 4.02 (s, 2H), 3.1212.98 (m+m, 4H), 1.79/1.56 (m+m, 4H)

¹³C-NMR (125 MHz, dmso-d6) δ ppm 159.2, 153, 145.3, 122.8, 116.6, 53.9, 39.5, 31.4

### Step 3: Example 108

Using General Procedure 6 starting from 5-amino-6-(4-fluorophenoxy)-3-[(4-hydroxy-4-piperidyl)methyl]pyrimidin-4-one hydrochloride and Preparation R8a as reagents, EXAMPLE 108 was obtained. HRMS calculated for C₃₇H₄₀FN₅O₅: 653.3013; found 654.308 ((M+H)⁺ form).

### 5-amino-6-(4-fluorophenoxy)-3-[[1-[(3R,4R)-1-13-(2-fluorophenyl)oxetan-3-yl]-3-phenyl-piperidine-4-carbonyl]-4-hydroxy-4-piperidyl]methyl]pyrimidin-4-one (Example 109)

Using General Procedure 4 starting from Preparation R4h and Preparation R10b as reagents, EXAMPLE 109 was obtained. HRMS calculated for C₃₇H₃₉F₂N₅O₅: 671.2919; found 672.2981 ((M+H)⁺ form).

### 5-amino-6-(4-fluorophenoxy)-3-[[1-[(3R,4R)-1-[3-(4-fluorophenyl)oxetan-3-yl]-3-phenyl-piperidine-4carbonyl]-4-hydroxy-4-piperidyl]methyl]pyrimidin-4-one (Example 110)

Using General Procedure 4 starting from Preparation R4h and Preparation R10c as reagents, EXAMPLE 110 was obtained. HRMS calculated for C₃₇H₃₉F₂N₅O₅: 671.2919: found 672.2996 ((M+H)⁺ form).

### 5-amino-6-(4-fluorophenoxy)-3-[[4-hydroxy-1-[(3R,4R)-1-(3-methyloxetan-3-yl)-3-phenyl-piperidine-4-carbonyl]-4-piperidyl]methyl]pyrimidin-4-one (Example 111)

Using General Procedure 4 starting from Preparation R4h and Preparation R10d as reagents, EXAMPLE 111 was obtained. HRMS calculated for C₃₂H₃₈FN₅O₅: 591.2857; found 592.2925 ((M+H)⁺ form).

### 5-amino-6-(4-fluorophenoxy)-3-[[4-hydroxy-1-[(3R,4R)-1-(3-isopropyloxetan-3-yl)-3-phenyl-piperidine-4-carbonyl]-4-piperidyl]methyl]pyrimidin-4-one (Example 112)

Using General Procedure 4 starting from Preparation R4h and Preparation R10e as reagents, EXAMPLE 112 was obtained. HRMS calculated for C₃₄H₄₂FN₅O₅: 619.317; found 620,324 ((M+H)⁺ form).

### 5-amino-3-[[4-hydroxy-1-[(3R,4R)-1-(3-methyloxetan-3-yl)-3-phenyl-piperidine-4-carbonyl]-4-piperidyl]methyl]-6-phenoxy-pyrimidin-4-one (Example 113)

Using General Procedure 4 starting from Preparation R4g and Preparation R10d as reagents, EXAMPLE 113 was obtained. HRMS calculated for C₃₂H₃₉N₅O₅: 573.2951; found 574.302 ((M+H)⁺ form).

### 5-amino-3-[[1-[(3R4R)-[3-(2-fluorophenyl)oxetan-3-yl-3-phenyl-piperidine-4-carbonyl]-4-hydroxy-4-piperidyl]methyl]-6-phenoxy-pyrimidin-4-one (Example 114)

Using General Procedure 4 starting from Preparation R4g and Preparation R10e as reagents, EXAMPLE 114 was obtained. HRMS calculated for C₃₇H₄₀FN₅O₅: 653.3013: found 654.3089 ((M+H)⁺ form).

### 5-amino-3-[[1-[(3R,4R)-1-[3-(4-fluorophenyl)oxetan-3-yl]-3-phenyl-piperidine-4-carbonyl]-4-hydroxy-4-piperidyl]methyl-6-phenoxy-pyrimidin-4-one (Example 115)

Using General Procedure 4 starting from Preparation R4g and Preparation R10c as reagents. EXAMPLE 115 was obtained. HRMS calculated for C₃₇H₄₀FN₅O₅: 653.3013; found 654.3086 ((M+H)⁺ form).

### 5-amino-6-(4-fluorophenoxy)-3-[(4-hydroxy-1-((3R,4R)-3-phenyl-1-[3-(piperidin-3-yl)benzoyl]piperidine-4-carbonyl}piperidin-4-yl)methyl]pyrimidin-4(3H)-one (Example 116)

Using General Procedure 6 starting from EXAMPLE 21 and 3-(I-tert-butoxycarbonyl-3-piperidyl)benzoic acid as reagents, tert-butyl 3-[3-[(3R,4R)-4-[4-[[5-amino-4-(4-fluorophenoxy)-6-oxo-pyrimidin-1-yl]methyl]-4-hydroxy-piperidine-1-carbonyl]-3-phenyl-piperidine-1-carbonyl]phenyl]piperidine-1-carboxylate was obtained as a crude product and was directly reacted using General Procedure 5 to give EXAMPLE 116. HRMS calculated for C₄₀H₄₅FN₆O₅: 708.3436: found 709.35076 ((M+H)⁺ form).

### 5-amino-6-(4-fluorophenoxy)-3-[[4-hydroxy-1-(3R,4R)-1-[[5-(6-methyl-3-pyridyl)-2-thienyl]sulfonyl]-3-phenyl-piperidine-4-carbonyl]-4-piperidyl]methyl]pyrimidin-4-one (Example 117)

Using General Procedure 11 starting from EXAMPLE 59 and (6-methyl-3-pyridyl)boronic acid as reagents, EXAMPLE 117 was obtained. HRMS calculated for C₃₈H₃₉FN₆O₆S₂: 758.2357; found 759.2423 ((M+H)⁺ form).

### 5-amino-6-(4-fluorophenoxy)-3-[[4-hydroxy-1-[(3R,4R)-3-phenyl-[[5-(2thienyl)-2-thienyl]sulfonyl]piperidine-4-carbonyl]-4-piperidyl]methyl]pyrimidin-4-one (Example 118)

Using General Procedure 11 starting from EXAMPLE 59 and 2-thienylboronic acid as reagents, EXAMPLE 118 was obtained. FIRMS calculated for C₃₆H₃₆FN₅O₆S₃: 749.1812; found 750.1883 ((M+H)⁺ form).

### 5-amino-3-{1(4S)-3,3-difluoro-4-hydroxy-1-{(3R,4R)-1-[4-methyl-2-(6-methylpyridin-3-yl)-1,3-thiazole-5-carbonyl]-3-phenylpiperidine-4-carbonyl]piperidin-4-yl]methyl)-6-phenoxypyrimidin-4(3H)-one (Example 119)

Using General Procedure 6 starting from EXAMPLE 12 and 4-methyl-2-(6-methyl-3-pyridyl)thiazole-5-carboxylic acid as reagents, EXAMPLE 119 was obtained. HRMS calculated for C₃₉H₃₉F₂N₇O₅S: 755.2701; found 756.27677 ((M+H)⁺ form).

### 5-amino-3-({(4S)-3,3-difluoro-4-hydroxy-1-[(3R,4R)-1-(5-methyl-1-phenyl-1H-pyrazole-3-carbonyl)-3phenylpiperidine-4-carbonyl]piperidin-4-yl)methyl)-6(4-fluorophenoxy)pyrimidin-4(3H)-one (Example 120)

Using General Procedure 6 starting from EXAMPLE 11 and 5-methyl-1-phenyl-pyrazole-3-carboxylic acid as reagents, EXAMPLE 120 was obtained. HRMS calculated for C₃₉H₃₈F₃N₇O₅: 741.2886; found 742.2948 ((M+H)⁺ form).

### 5-amino-3-((1-{(3R,4R)-1-[3-fluoro-5-(6-methylpyridin-3-yl)benzoyl]-3-phenylpiperidine-4-carbonyl-4-hydroxypiperidin-4-yl)methyl]-6-(4-fluorophenoxy)pyrimidin-4(3H)-one (Example 121)

Using General Procedure 11 starting from EXAMPLE 98 and (6-methyl-3-pyridyl)boronic acid as reagents, EXAMPLE 121 was obtained. HRMS calculated for C₄₁H₄₀F₂N₆O₅: 734.3028; found 735.3104 ((M+H)⁺ form).

### 5-amino-6-(4-fluorophenoxy)-3-[(4-hydroxy-1-{(3R,4R)-3-phenyl-1-[5-(trifluoromethyl)pyridine-3-carbonyl]piperidine-4-carbonyl}piperidin-4-yl)methyl]pyrimidin-4(3H)-one (Example 122)

Using General Procedure 6 starting from EXAMPLE 21 and 5-(trifluoromethyl)pyridine-3-carboxylic acid as reagents, EXAMPLE 122 was obtained. HRMS calculated for C₃₅H₃₄FN₆O₅: 694.2527; found 695.2593 ((M+H)⁺ form).

### 6-[(3R,4R)-4-(4-({5-amino-4-(4-fluorophenoxy)-6-oxopyrimidin-1(6H)-yl]methyl)-4-hydroxypiperidine-1-carbonyl)-3-phenylpiperidine-1-carbonyl]-3-methylquinazolin-4(3H)-one (Example 123)

Using General Procedure 6 starting from EXAMPLE 21 and 3-methyl-4-oxo-quinazoline-6-carboxylic acid as reagents, EXAMPLE 123 was obtained. HRMS calculated for C₃₈H₃₈FN₇O₆: 707.2867; found 708.2929 ((M+H)⁺ form).

### 5-amino-3-[[1-[(3R,4R)-1-[3-fluoro-5-[16-(trifluoromethyl)-3-pyridyl]thiophene-2-carbonyl]-3-phenyl-piperidine-4-carbonyl]-4-hydroxy-4-piperidyl]methyl]-6-[3-(trifluoromethyl)phenoxy]pyrimidin-4-one (Example 124)

Using General Procedure 11 starting from EXAMPLE 99 and [6-(trifluoromethyl)-3-pyridyl]boronic acid as reagents, EXAMPLE 124 was obtained. HRMS calculated for C₄₀H₃₅F₇N₆O₅S: 844.2278; found 845.2348 ((M+H)⁺ form).

### 5-amino-3-[[4-hydroxy-1-[(3R,4R)-1-[4-methyl-2-[6-(trifluoromethyl)-3-pyridyl]thiazole-5-carbonyl]-3-phenyl-piperidine-4-carbonyl]-4-piperidyl]methyl]-6-[3-(trifluoromethyl)phenoxy]pyrimidin-4-one (Example 125)

Using General Procedure 1i starting from EXAMPLE 100 and 16-(trifluoromethyl)-3-pyridyl]boronic acid as reagents, EXAMPLE 125 was obtained. HRMS calculated for C₄₀H₃₇F₆N₇O₅S: 841,2481; found 842.255 ((M+H)⁺ form).

### 5-amino-3-[[1-[(3R,4R)-1-[2-[6-(dimethylamino)-3-pyridyl]-4-methyl-thiazole-5-carbonyl]-3-phenyl-piperidine-4-carbonyl]-4-hydroxy-4-piperidyl]methyl-6-(3-(trifluoromethyl)phenoxy]pyrimidin-4-one (Example 126)

Using General Procedure 11 starting from EXAMPLE 100 and [6-(dimethylamino)-3-pyridyl]boronic acid as reagents, EXAMPLE 126 was obtained. HRMS calculated for C₄₁H₄₃F₃N₈O₅S: 816.3029: found 817.3114 ((M+H)⁺ form).

### 5-amino-3-[[1-[(3R,4R)-1-(3-chloro-1-methyl-indole-2-carbonyl)-3-phenyl-piperidine-4-carbonyl]-4-hydroxy-4-piperidyl]methyl]-6-(4-fluorophenoxy)pyrimidin-4-one (Example 127)

Using General Procedure 6 starting from EXAMPLE 21 and 3-chloro-1-methyl-indole-2-carboxylic acid as reagents. EXAMPLE 127 was obtained, HRMS calculated for C₃₈H₃₈ClFN₆O₅: 712.2576: found 713.2653 ((M+H)⁺ form).

### (3R,4R)-4-[4-[[5-amino-4-(4-fluorophenoxy)-6-oxo-pyrimidin-1-yl]methyl]-4-hydroxy-piperidine-1-carbonyl]-3-phenyl-N-[2-(trifluoromethyl)phenyl]piperidine-1-carboxamide (Example 128)

Using General Procedure 8 starting from EXAMPLE 21 and 2-(trifluoromethyl)aniline as reagents, EXAMPLE 128 was obtained. HRMS calculated for C₃₆H₃₆F₄N₆O₅: 708.2683: found 709.2756 ((M+H)⁺ form),

### 5-amino-3-[[1-[(3R,4R)-1-(3,5-difluorobenzoyl)-3-phenyl-piperidine-4-carbonyl]-4-hydroxy-4-piperidyl]methyl]-6-(4-fluorophenoxy)pyrimidin-4-one (Example 129)

Using General Procedure 6 starting from EXAMPLE 21 and 3,5-difluorobenzoic acid as reagents. EXAMPLE 129 was obtained. HRMS calculated for C₃₅H₃₄F₃N₅O₅: 661.2512; found 662.2579 ((M+H)⁺ form).

### 5-amino-3-[[1-[(3R,4R)-1-(3,5-dichlorobenzoyl)-3-phenyl-piperidine-4-carbonyl-4-hydroxy-4-piperidyl]methyl]-6-(4-fluorophenoxy)pyrimidin-4-one (Example 130)

Using General Procedure 6 starting from EXAMPLE 21 and 3,5-dichlorobenzoic acid as reagents, EXAMPLE 130 was obtained. HRMS calculated for C₃₅H₃₄Cl₂FN₅O₅: 693.1921; found 694.1991 ((M+H)⁺ form).

### 5-amino-6-4-(aminomethyl)phenoxy]-3-[[4-hydroxy-1-[(3R,4R)-1-(4-methyl-2-(6-methyl-3-pyridyl)thiazole-5-carbonyl]-3-phenyl-piperidine-4-carbonyl]-4-piperidyl]methyl]pyrimidin-4-one (Example 131)

Using General Procedure 4 starting from Preparation R4j and Preparation R5a as reagents, tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate was obtained.

Using General Procedure 5 starting from tert-butyl (3R,4R)-4-[4-[[5-amino-4-(4-cyanophenoxy)-6-oxo-pyrimidin-1-yl}methyl)-4-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile.

Using General Procedure 6 starting from 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile and 4-methyl-2-(6-methyl-3-pyridyl)thiazole-5-carboxylic acid as reagents, 4-[5-amino-1-[[4-hydroxy-1-[(3R,4R)-1-[4-methyl-2-(6-methyl-3-pyridyl)thiazole-5-carbonyl]-3-phenyl-piperidine-4-carbonyl]-4-piperidyl]methyl]-6-oxo-pyrimidin-4-yl]oxybenzonitrile was obtained.

Using General Procedure 2 starting from 4-{5-amino-1-[[4-hydroxy-1-[(3R,4R)-1-[4-methyl-2-(6-methyl-3-pyridyl)thiazole-5-carbonyl-3-phenyl-piperidine-4-carbonyl]-4-piperidyl]methyl]-6-oxo-pyrimidin-4-yl]oxybenzonitrile as reagent. EXAMPLE 131 was obtained. HRMS calculated for C₄₀H₄₄N₈O₅S: 748.3156: found 749.32279 ((M+H)⁺ form).

### 5-amino-6-(4-fluorophenoxy)-3-[[4-hydroxy-1-[(3R,4R)-3-phenyl-1-(I-phenylcyclopropanecarbonyl)piperidine-4-carbonyl]-4-piperidyl]methylpyrimidin-4-one (Example 132)

Using General Procedure 6 starting from EXAMPLE 21 and 1-phenylcyclopropanecarboxylic acid as reagents, EXAMPLE 132 was obtained. HRMS calculated for C₃₈H₄₀FN₅O₅: 665.3013; found 666.3087 ((M+H)⁺ form).

### 5-amino-3-[[1-[(3R,4R)-1-(5-chloropyridine-3-carbonyl)-3-phenyl-piperidine-4-carbonyl]-4-hydroxy-4-piperidyl]methyl]-6-(4-fluorophenoxy)pyrimidin-4-one (Example 133)

Using General Procedure 6 starting from EXAMPLE 21 and 5-chloropyridine-3-carboxylic acid as reagents. EXAMPLE 133 was obtained. HRMS calculated for C₃₄H₃₄ClFN₆O₅: 660.2263; found 661.23313 ((M+H)⁺ form).

### 5-amino-6-(4-fluorophenoxy)-3-[[1-[(3R,4R)-1-(5-fluoropyridine-3-carbonyl)-3-phenyl-piperidine-4-carbonyl]-4-hydroxy-4-piperidyl]methyl]pyrimidin-4-one (Example 134)

Using General Procedure 6 starting from EXAMPLE 21 and 5-fluoropyridine-3-carboxylic acid as reagents. EXAMPLE 134 was obtained, HRMS calculated for C₃₄H₃₄F₂N₆O₅: 644.2559; found 645.26271 ([M+H]⁺ form).

### tert-butyl (3R,4R)-4-[(4S)-4-{15-amino-6-(4-fluoro-3-methoxy-phenoxy)pyrimidin-4-yl]oxymethyl]-3,3-difluoro-4-hydroxy-piperidine-1-carbonyl]-3-phenyl-piperidine-1-carboxylate (Example 135)

Using General Procedure 4 starting from Preparation R4d and Preparation R5b as reagents, the enantiomers were separated by chiral chromatography to give EXAMPLE 135. HRMS calculated for C₃₄H₄₀F₃N₅O₇: 687.288; found 710.2764 ([M+Na[⁺ form).

### [(4S)-4-[[5-amino-6-4-fluoro-3-methoxy-phenoxy)pyrimidin-4-yl]oxymethyl]-3,3-difluoro-4-hydroxy-1-piperidyl]-[(3R,4R)-3-phenyl-4-piperidyl]methanone hydrochloride (Example 136)

Using General Procedure 5 starting from EXAMPLE 135 as reagent, EXAMPLE 136 was obtained. HRMS calculated for C₃₉H₃₂F₃N₅O₅: 587.2355; found 588.2426 ([M+H]⁺ form).

### tert-butyl (3R,4R)-4-[4-[[5-amino-6-oxo-4-(3-pyridyloxy)pyrimidin-1-yl]methyl]-4-hydroxy-piperidine-1-carbonyl]-3-phenyl-piperidine-1-carboxylate (Example 137)

Using General Procedure 4 starting from Preparation R4k and Preparation R5a as reagents. EXAMPLE 137 was obtained. HRMS calculated for C₃₂H₄₀N₆O₆: 604.3009; found 605.3079 ([M+H]⁺ form).

### 5-amino-6-(4-fluorophenoxy)-3-[4-hydroxy-1-(3R,4R)-1-[3-(o-tolyl)oxetan-3-yl]-3-phenyl-piperidine-4-carbonyl]-4-piperidyl]methyl]pyrimidin-4-one (Example 138)

Using General Procedure 6 starting from 5-amino-6-(4-fluorophenoxy)-3-[(4-hydroxy-4-piperidyl)methyl]pyrimidin-4-one hydrochloride and Preparation R8f as reagents, EXAMPLE 138 was obtained. HRMS calculated for C₃₈H₄₂FN₅O₅: 667.317; found 668.3237 ([M+H]⁺ form).

### 5-amino-6-(4-fluorophenoxy)-3-[[4-hydroxy-1-[(3R,4R)-1-[3-(2-methoxyphenyl)oxetan-3-yl]-3-phenyl-piperidine-4-carbonyl]-4-piperidyl]methyl]pyrimidin-4-one (Example 139)

Using General Procedure 6 starting from 5-amino-6-(4-fluorophenoxy)-3-[(4-hydroxy-4-piperidyl)methyl]pyrimidin-4-one hydrochloride and Preparation R8g as reagents. EXAMPLE 139 was obtained. HRMS calculated for C₃₈H₄₂FN₅O₆: 683.3119; found 684.3175 ([M+H]⁺ form).

### 5-amino-3-[[1-((3R,4R)-1-[3-(2-chlorophenyl)oxetan-3-yl]-3-phenyl-piperidine-4-carbonyl]-4-hydroxy-4-piperidyl]methyl]-6-(4-fluorophenoxy)pyrimidin-4-one (Example 140)

Using General Procedure 6 starting from 5-amino-6-(4-fluorophenoxy)-3-[(4-hydroxy-4-piperidyl)methyl]pyrimidin-4-one hydrochloride and Preparation R8n as reagents. EXAMPLE 140 was obtained. HRMS calculated for C₃₇H₃₉ClFN₅O₅: 687,2624; found 688.2687 ([M+H]⁺ form).

### tert-butyl (3R,4R)-4-[(4S)-4-[[5-amino-6-oxo-4-[3-(trifluoromethyl)phenoxy]pyrimidin-1-yl]methyl]-3,3-difluoro-4-hydroxy-piperidine-1-carbonyl]-3-phenyl-piperidine-1-carboxylate (Example 141)

Using General Procedure 4 starting from Preparation R4e and Preparation R5b as reagents, the enantiomers were separated by chiral chromatography to give EXAMPLE 141. HRMS calculated for C₃₄H₃₈F₅N₅O₆: 707.2742: found 730.2632 ([M+Na[⁺ form).

### 5-amino-3-[[(4S)-3,3-difluoro-4-hydroxy-1-[(3R,4R)-3-phenylpiperidine-4-carbonyl]-4-piperidyl]methyl]-6-[3-(trifluoromethyl)phenoxy]pyrimidin-4-one, hydrochloride (Example 142)

Using General Procedure 5 starting from EXAMPLE 141 as reagent, EXAMPLE 142 was obtained. HRMS calculated for C₂₉H₃₀F₅N₅O₄: 607.221R: found 608.2283 ([M+H]⁺ form).

### 5-amino-3-[[(4S)-3,3-difluoro-4-hydroxy-1-[(3R,4R)-1-[(2-methylpyrimidin-4-yl)methyl]-3-phenyl-piperidine-4-carbonyl]-4-piperidyl]methyl]-6-(3-(trifluoromethyl)phenoxy]pyrimidin-4-one (Example 143)

Using General Procedure 9 starting from EXAMPLE 142 and 4-(chloromethyl)-2-methyl-pyrimidine as reagents, EXAMPLE 143 was obtained. HRMS calculated for C₃₅H₃₆F₅N₇O₄: 713,2749; found 714.2814 ([M+H]⁺ form).

### 5-amino-3-{[(4S)-3,3-difluoro-4-hydroxy-1-[(3R,4R)-1-((5-methylpyrazin-2-yl)methyl]-3-phenyl-piperidine-4-carbonyl]-4-piperidyl]methyl]-6-[3-(trifluoromethyl)phenoxy]pyrimidin-4-one (Example 144)

Using General Procedure 9 starting from EXAMPLE 142 and 2-(chloromethyl)-5-methyl-pyrazine hydrochloride as reagents, EXAMPLE 144 was obtained. HRMS calculated for C₃₅H₃₆F₅N₇O₄: 713.2749: found 714.2813 ([M+H]⁺ form).

### tert-butyl (3R,4R)-4-[(4S)-4-[[5-amino-4-(4-chloro-3-fluoro-phenoxy)-6-oxo-pyrimidin-1-yl]methyl]-3,3-difluoro-4-hydroxy-piperidine-1-carbonyl)-3-phenyl-piperidine-1-carboxylate (Example 145)

Using General Procedure 4 starting from Preparation R4c and Preparation R5b as reagents, the enantiomers were separated by chiral chromatography to give EXAMPLE 145. HRMS calculated for C₃₅H₃₇ClF₃N₅O₆: 691.2385; found 714.2275 ([M+Na]⁺ form).

### 5-amino-6-(4-chloro-3-fluoro-phenoxy)-3-[[(4S)-3,3-difluoro-4-hydroxy-1-(3R,4R)-3-phenylpiperidine-4-carbonyl]-4-piperidyl]methyl]pyrimidin-4-one, hydrochloride (Example 146)

Using General Procedure 5 starting from EXAMPLE 145 as reagent, EXAMPLE 146 was obtained. HRMS calculated for C₂₈H₂₉ClF₃N₅O₄: 591.186; found 592.1923 ([M+H]⁺ form).

### 5-amino-6-(4-chloro-3-fluoro-phenoxy)-3-[[(4S)-3,3-difluoro-4-hydroxy-1-(3R,4R)-1-[(2-methylpyrimidin-4-yl)methyl]-3-phenyl-piperidine-4-carbonyl]-4-piperidyl]methyl]pyrimidin-4-one (Example 147)

Using General Procedure 9 starting from EXAMPLE 146 and 4-(chloromethyl)-2-methyl-pyrimidine as reagents, EXAMPLE 147 was obtained. HRMS calculated for C₃₄H₃₅ClF₃N₇O₄: 697.2391; found 698.2453 ([M+H]⁺ form).

### 5-amino-6-(4-chloro-3-fluoro-phenoxy)-3-[[(4S)-3,3-difluoro-4-hydroxy-1-[(3R,4R)-1-[(5-methylpyrazin-2-yl)methyl]-3-phenyl-piperidine-4-carbonyl]-4-piperidyl]methyl]pyrimidin-4-one (Example 148)

Using General Procedure 9 starting from EXAMPLE 146 and 2-(chloromethyl)-5-methyl-pyrazine hydrochloride as reagents, EXAMPLE 148 was obtained. HRMS calculated for C₃₄H₃₅ClF₃N₇O₄: 697.2391: found 698.2447 ([M+H]⁺ form).

### 5-amino-3-[[(4S)-3,3-difluoro-4-hydroxy-1-[(3R,4R)-1-[(2-methylpyrimidin-4-yl)methyl]-3-phenyl-piperidine-4-carbonyl]-4-piperidylmethyl-6-(4-fluoro-3-methoxy-phenoxy)pyrimidin-4-one (Example 149)

Using General Procedure 9 starting from EXAMPLE 136 and 4-(chloromethyl)-2-methyl-pyrimidine as reagents. EXAMPLE 149 was obtained, HRMS calculated for C₃₅H₃₈F₃N₇O₅: 693.2886; found 694.2959 ([M+H]⁺ form).

### 5-amino-6-(4-fluorophenoxy)-3-[[4-hydroxy-1-[(3R,4R)-1-[4-methyl-2-(2-thienyl)thiazole-5-carbonyl]-3-phenyl-piperidine-4-carbonyl]-4-piperidyl]methyl]pyrimidin-4-one (Example 150)

Using General Procedure 11 starting from EXAMPLE 29 and 2-thienylboronic acid as reagents, EXAMPLE 150 was obtained. HRMS calculated for C₃₇H₃₇FN₆O₅S₂: 728.2251; found 729.2313 ([M+H]⁺ form).

### 5-amino-3-[[4-hydroxy-1-[(3R,4R)-3-phenylpiperidine-4-carbonyl]-4-piperidyl]methyl]-6-(3-pyridyloxy)pyrimidin-4-one, hydrochloride (Example 151)

Using General Procedure 5 starting from EXAMPLE 137 as reagent, EXAMPLE 151 was obtained. HRMS calculated for C₂₇H₃₂N₆O₄: 504.2485; found 505.2555 ([M+H[⁺ form).

### 5-amino-6-(4-fluorophenoxy)-3-[[4-hydroxy-1-[(3R,4R)-3-phenyl-1-[3-[3-(trifluoromethyl)phenyl]oxetan-3-yl]piperidine-4-carbonyl]-4-piperidyl]methyl]pyrimidin-4-one (Example 152)

Using General Procedure 6 starting from 5-amino-6-(4-fluorophenoxy)-3-[(4-hydroxy-4-piperidyl)methyl]pyrimidin-4-one hydrochloride and Preparation R8h as reagents, EXAMPLE 152 was obtained. HRMS calculated for C₃₈H₃₉F₄N₅O₅: 721.2888; found 722,2945 ([M+H]⁺ form).

### 5-amino-6-(4-fluorophenoxy)-3-[[1-[(3R,4R)-1-[3-(3-fluorophenyl)oxetan-3-yl]-3-phenyl-piperidine-4-carbonyl]-4-hydroxy-4-piperidyl]methyl]pyrimidin-4-one (Example 153)

Using General Procedure 6 starting from 5-amino-6-(4-fluorophenoxy)-3-[(4-hydroxy-4-piperidyl)methyl]pyrimidin-4-one hydrochloride and Preparation R8i as reagents, EXAMPLE 153 was obtained. HRMS calculated for C₃₇H₃₉F₂N₅O₅: 671.2919; found 672.2976 ([M+H]⁺ form).

### 5-amino-3-1(4S)-3,3-difluoro-4-hydroxy-1-[(3R,4R)-1-[(5-methylpyrazin-2-yl)methyl]-3-phenyl-piperidine-4-carbonyl}-4-piperidyl]methyl]-6-(4-fluoro-3-methoxy-phenoxy)pyrimidin-4-one (Example 154)

Using General Procedure 9 starting from EXAMPLE 136 and 2-(chloromethyl)-5-methyl-pyrazine hydrochloride as reagents, EXAMPLE 154 was obtained. HRMS calculated for C₃₅H₃₈F₃N₇O₅: 693,2886; found 694.2949 ([M+H]⁺ form).

### 5-amino-6-(4-fluorophenoxy)-3-[[4-hydroxy-1-[(3R,4R)-3-phenyl-1-[3-(p-tolyl)oxetan-3-yl{piperidine-4-carbonyl]-4-piperidyl]methyl]pyrimidin-4-one (Example 155)

Using General Procedure 6 starting from 5-amino-6-(4-fluorophenoxy)-3-[(4-hydroxy-4-piperidyl)methyl]pyrimidin-4-one hydrochloride and Preparation R8l as reagents, EXAMPLE 155 was obtained. HRMS calculated for C₃₈H₄₂FN₅O₅: 667.317; found 668,3237 ([M+H]⁺ form).

### 5-amino-6-(4-fluorophenoxy)-3-[[4-hydroxy-1-[(3R,4R)-1-[3-(4-methoxyphenyl)oxetan-3-yl]-3-phenyl-piperidine-4-carbonyl]-4-piperidyl]methyl]pyrimidin-4-one (Example 156)

Using General Procedure 6 starting from 5-amino-6-(4-fluorophenoxy)-3-[(4-hydroxy-4-piperidyl)methyl]pyrimidin-4-one hydrochloride and Preparation R8m as reagents. EXAMPLE 156 was obtained. HRMS calculated for C₃₈H₄₂FN₅O₆: 683.3119: found 684.3186 ([M+H]⁺ form).

### 5-amino-3-[[(4S)-3,3-difluoro-4-hydroxy-1-[(3R,4R)-1-(5-methylpyridine-3-carbonyl)-3-phenyl-piperidine-4-carbonyl]-4-piperidyl]methyl]-6-(4-fluorophenoxy)pyrimidin-4-one (Example 157)

Using General Procedure 6 starting from EXAMPLE 11 and 5-methylpyridine-3-carboxylic acid as reagents, EXAMPLE 157 was obtained. HRMS calculated for C₃₅H₃₅F₃N₆O₅: 676.2621; found 677.2682 ([M+H]⁻ form),

### 5-amino-3-[[1-[(3R,4R)-1-(2-cyclohexyl-4-methyl-thiazole-5-carbonyl)-3-phenyl-piperidine-4-carbonyl-4-hydroxy-4-piperidyl]methyl]-6-(4-fluorophenoxy)pyrimidin-4-one (Example 158)

Using General Procedure 6 starting from EXAMPLE 21 and 2-cyclohexyl-4-methyl-thiazole-5-carboxylic acid as reagents, EXAMPLE 158 was obtained. HRMS calculated for C₃₉H₄₅FN₆O₅S: 728.3156; found 729.3221 ([M+H]⁺ form).

### 5-amino-6-(4-fluorophenoxy)-3-[[4-hydroxy-1-[(3R,4R)-1-[3-(6-methyl-3-pyridyl)phenyl]sulfonyl-3-phenyl-piperidine-4-carbonyl]-4-piperidyl]methyl]pyrimidin-4-one (Example 159)

Using General Procedure 11 starting from EXAMPLE 69 and (6-methyl-3-pyridyl)boronic acid as reagents, EXAMPLE 159 was obtained. HRMS calculated for C₄₀H₄₁FN₆O₆S: 752.2792; found 753.2871 ([M+H]⁺ form).

### 5-amino-3-[[(4S)-1-[(3R,4R)-1-[(2-chloropyrimidin-4-yl)methyl]-3-phenyl-piperidine-4-carbonyl-3,3-difluoro-4-hydroxy-4-piperidyl]methyl]-6-(4-fluorophenoxy)pyrimidin-4-one (Example 160)

Using General Procedure 9 starting from EXAMPLE 11 and 2-chloro-4-(chloromethyl)pyrimidine as reagents. EXAMPLE 160 was obtained. HRMS calculated for C₃₃H₃₃ClF₃N₇O₄: 683.2234; found 684.2304 ([M+H]⁺ form).

### 5-amino-3-[[1-[(3R,4R)-1-(2-cyclopentyl-4-methyl-thiazole-5-carbonyl)-3-phenyl-piperidine-4-carbonyl]-4-hydroxy-4-piperidyl]methyl]-6-(4-fluorophenoxy)pyrimidin-4-one (Example 161)

Using General Procedure 6 starting from EXAMPLE 21 and 2-cyclopentyl-4-methyl-thiazole-5-carboxylic acid as reagents, EXAMPLE 161 was obtained. HRMS calculated for C₃₈H₄₃FN₆O₅S: 714.3; found 715.3061 ([M+H]⁺ form).

### 5-amino-3-[[1-[(3R,4R)-1-(3-chloro-5-methyl-benzoyl)-3-phenyl-piperidine-4-carbonyl]-4-hydroxy-4-piperidyl]methyl-6-(4-fluorophenoxy)pyrimidin-4-one (Example 162)

Using General Procedure 6 starting from EXAMPLE 21 and 3-chloro-5-methyl-benzoic acid as reagents, EXAMPLE 162 was obtained. HRMS calculated for C₃₆H₃₇ClFN₅O₅: 673.2467; found 674.2543 ([M+H]⁺ form).

### 5-amino-6-(4-fluorophenoxy)-3-[[4-hydroxy-1-[(3R,4R)-3-phenyl-1-(5-pyrrol-1-ylpyridine-3-carbonyl)piperidine-4-carbonyl]-4-piperidyl]methyl]pyrimidin-4-one (Example 163)

Using General Procedure 6 starting from EXAMPLE 21 and 5-pyrrol-1-ylpyridine-3-carboxylic acid as reagents. EXAMPLE 163 was obtained. HRMS calculated for C₃₈H₃₈FN₇O₅: 691.2919: found 692.3003 ([M+H]⁺ form).

### 5-amino-3-[[(4S)-3,3-difluoro-4-hydroxy-1-[(3R,4R)-1-[[6-(6-methyl-3-pyridyl)pyrimidin-4-yl}methyl]-3-phenyl-piperidine-4-carbonyl]-4-piperidyl]methyl]-6-(4-fluorophenoxy)pyrimidin-4-one (Example 164)

Using General Procedure 9 starting from EXAMPLE 11 and 4-chloro-6-(chloromethyl)pyrimidine, 5-amino-3-[[(4S)-1-[(3R,4R)-1-[(6-chloropyrimidin-4-yl)methyl]-3-phenyl-piperidine-4-carbonyl]-3,3-difluoro-4-hydroxy-4-piperidyl]methyl]-6-(4-fluorophenoxy)pyrimidin-4-one was obtained as a crude product and was directly reacted with (6-methyl-3-pyridyl)boronic acid using General Procedure 11 to give EXAMPLE 164. HRMS calculated for C₃₉H_{39F}3N₈O₄: 740.3046; found 741.3119 ([M+H]⁺ form).

### 5-amino-3-[[1-[(3R,4R)-1-(2-cyclobutyl-4-methyl-thiazole-5-carbonyl)-3-phenyl-piperidine-4-carbonyl]-4-hydroxy-4-piperidyl]methyl]-6-(4-fluorophenoxy)pyrimidin-4-one (Example 165)

Using General Procedure 6 starting from EXAMPLE 21 and 2-cyclobutyl-4-methyl-thiazole-5-carboxylic acid as reagents, EXAMPLE 165 was obtained. HRMS calculated for C₃₇H₄₁FN₆O₅S: 700.2843; found 701.2913 ([M+H]⁺ form).

### tert-butyl (3R,4R)-4-(4S)-4-[[5-amino-4-(4-chlorophenoxy)-6-oxo-pyrimidin-1-yl]methyl]-3,3-difluoro-4-hydroxy-piperidine-1-carbonyl]-3-phenyl-piperidine-1-carboxylate (Example 166)

Using General Procedure 4 starting from Preparation R4a and Preparation R5b as reagents, the enantiomers were separated by chiral chromatography to give EXAMPLE 166. HRMS calculated for C₃₃H₃₈ClF₂N₅O₆: 673.2479; found 696,2384 ([M+Na[⁺ form).

### 5-amino-6-(4-chlorophenoxy)-3-[(4S)-3,3-difluoro-4-hydroxy-1-[(3R,4R)-3-phenylpiperidine-4-carbonyl]-4-piperidyl]methyl]pyrimidin-4-one (Example 167)

Using General Procedure 5 starting from EXAMPLE 166 as reagent, the crude product was purified by preparative LC (on C-18 Gemini-NX 5 μm column, 5 mM aqueous NH₄HCO₃-MeCN, gradient) and solvent was evaporated to give EXAMPLE 167. HRMS calculated for C₂₈H₃₀ClF₂N₅O₄: 573.1954: found 574.2028 ([M+H]⁺ form).

### 5-amino-6-(4-chlorophenoxy)-3-[[(4S)-3,3-difluoro-4-hydroxy-1-(3R,4R)-1-[(2-methylpyrimidin-4-yl)methyl]-3-phenyl-piperidine-4-carbonyl]-4-piperidyl]methyl]pyrimidin-4-one (Example 168)

Using General Procedure 9 starting from EXAMPLE 167 and 4-(chloromethyl)-2-methyl-pyrimidine as reagents, EXAMPLE 168 was obtained. HRMS calculated for C₃₄H₃₆ClF₂N₇O₄: 679.2485: found 680.2556 ([M+H]⁺ form).

### 5-amino-3-(14-hydroxy-41-(3R,4R)-1-[[4-methyl-2-(6-methyl-3-pyridyl)thiazole-5-carbonyl]-3-phenyl-piperidine-4-carbonyl]4-piperidyl]methyl]-6-(3-pyridyloxy)pyrimidin-4-one (Example 169)

Using General Procedure 6 starting from EXAMPLE 151 and 4-methyl-2-(6-methyl-3-pyridyl)thiazole-5-carboxylic acid as reagents, EXAMPLE 169 was obtained. HRMS calculated for C₁₈H₄₀N₈O₅S: 720.2842. found 721.2914 ([M+H]⁺ form).

### 5-amino-3-[[1-[(3R,4R)-1-[3-(3-bromophenyl)oxetan-3-yl]-3-phenyl-piperidine-4-carbonyl]-4-hydroxy-4-piperidyl]methyl]-6-(4-fluorophenoxy)pyrimidin-4-one (Example 170)

Using General Procedure 6 starting from 5-amino-6-(4-fluorophenoxy)-3-[(4-hydroxy-4-piperidyl)methyl]pyrimidin-4-one hydrochloride and Preparation R8j as reagents, EXAMPLE 170 was obtained. HRMS calculated for C₃₇H₃₉BrFN₅O₅: 731.2119; found 732.2182 ([M+H]⁺ form).

### 5-amino-3-[[1- (3R,4R)-1-[3-(3-chlorophenyl)oxetan-3-yl]-3-phenyl-piperidine-4-carbonyl]-4-hydroxy-4-piperidyl]methyl]-6-(4-fluorophenoxy)pyrimidin-4-one (Example 171)

Using General Procedure 6 starting from 5-amino-6-(4-fluorophenoxy)-3-[(4-hydroxy-4-piperidyl)methyl]pyrimidin-4-one hydrochloride and Preparation R8k as reagents, EXAMPLE 171 was obtained. HRMS calculated for C₃₇H₃₉ClFN₅O₅: 687.2624; found 688.2697 ([M+H]⁺ form).

### 5-amino-6-(4-fluorophenoxy)-3-[[4-hydroxy-1-[(3R,4R)-1-[4-methyl-2-(3-piperidyl)thiazole-5-carbonyl]-3-phenyl-piperidine-4-carbonyl]-4-piperidyl]methyl]pyrimidin-4-one (Example 172)

Using General Procedure 6 starting from EXAMPLE 21 and 2-(l-tert-butoxycarbonyl-3-piperidyl)-4-methyl-thiazole-5-carboxylic acid, tert-butyl 3-[5-((3R,4R)-4-[4-[[5-amino-4-(4-fluorophenoxy)-6-oxo-pyrimidin-1-yl]methyl]-4-hydroxy-piperidine-1-carbonyl]-3-phenyl-piperidine-1-carbonyl]-4-methyl-thiazol-2-yl]piperidine-1-carboxylate was obtained as a crude product and was directly reacted using General procedure 5 to give EXAMPLE 172. HRMS calculated for C₃₈H₄₄FN₇O₅S: 729.3109; found 730.3178 ([M+H]⁺ form).

### tert-butyl (3R,4R)-4-[(4S)-4-[[5-amino-4-(3-chloro-5-methoxy-phenoxy)-6-oxo-pyrimidin-1-yl]methyl]-3,3-difluoro-4-hydroxy-piperidine-1-carbonyl]-3-phenyl-piperidine-1-carboxylate (Example 173)

Using General Procedure 4 starting from Preparation 4b and Preparation 5b as reagents, the enantiomers were separated by chiral chromatography to give EXAMPLE 173. HRMS calculated for C₃₄H₄₀ClF₂N₅O₇: 703.2584; found 604.2122 ([M+H-C₄H₈-CO₂]⁺ form)

### 5-amino-6-(3-chloro-5-methoxy-phenoxy)-3-[[(4S)-3,3-difluoro-4-hydroxy-1-1(3R,4R)-3-phenylpiperidine-4-carbonyl]-4-piperidyl]methyl]pyrimidin-4-one hydrochloride (Example 174)

Using General Procedure 5 starting from EXAMPLE 173 as reagent, EXAMPLE 174 was obtained. HRMS calculated for C₃₉H₃₂ClF₂N₅O₅: 603.206; found 604.213 ([M+H]⁺ form).

### 5-amino-6-(3-chloro-5-methoxy-phenoxy)-3-[[(4S)-3,3-difluoro-4-hydroxy-1-[(3R,4R)-1-[(2-methylpyrimidin-4-yl)methyl]-3-phenyl-piperidine-4-carbonyl]-4-piperidyl]methyl]pyrimidin-4-one (Example 175)

Using General Procedure 9 starting from EXAMPLE 174 and 4-(chloromethyl)-2-methyl-pyrimidine as reagents, EXAMPLE 175 was obtained. HRMS calculated for C₃₅H₃₈ClF₂N₇O₅: 709.2591; found 710.2668 ([M+H]⁺ form).

### 5-amino-3-[[(4S)-3,3-difluoro-4-hydroxy-1-((3R,4R)-1-{[2-(6-methyl-3-pyridyl)pyrimidin-4-yl]methyl]-3-phenyl-piperidine-4-carbonyl]-4-piperidyl]methyl]-6-(4-fluorophenoxy)pyrimidin-4-one (Example 176)

Using General Procedure 11 starting from EXAMPLE 160 and (6-methyl-3-pyridyl)boronic acid as reagents, EXAMPLE 176 was obtained. HRMS calculated for C₃₉H₃₉F₃N₅O₄: 740.3046; found 741.3102 ([M+H]⁺ form).

### 5-amino-3-[[(4S)-3,3-difluoro-4-hydroxy-1-[(3R,4R)-3-phenyl-1-[(2-phenylpyrimidin-4-yl)methyl]piperidine-4-carbonyl]-4-piperidyl]methyl]-6-(4-fluorophenoxy)pyrimidin-4-one (Example 177)

Using General Procedure 11 starting from EXAMPLE 160 and phenylboronic acid as reagents, EXAMPLE 177 was obtained, HRMS calculated for C₃₉H₃₈F₃N₇O₄: 725.2938: found 726.3025 ([M+H]⁺ form).

### 5-amino-6-(3-chloro-5-methoxy-phenoxy)-3-[[(4S)-3,3-difluoro-4-hydroxy-1-[(3R,4R)-1-[(5-methylpyrazin-2-yl)methyl]-3-phenyl-piperidine-4-carbonyl-4-piperidyl]methyl]pyrimidin-4-one (Example 178)

Using General Procedure 9 starting from EXAMPLE 174 and 2-(chloromethyl)-5-methyl-pyrazine hydrochloride as reagents, EXAMPLE 178 was obtained. HRMS calculated for C₃₅H₃₈ClF₂N₇O₅: 709.2591: found 710.2653 ([M+H]⁺ form).

### 5-amino-3-[[(4S)-3,3-difluoro-4-hydroxy-1-(3R,4R)-1-[4-methyl-2-(6-methyl-3-pyridyl)thiazole-5-carbonyl]-3-phenyl-piperidine-4-carbonyl]-4-piperidyl]methyl]-6-(4-fluoro-3-methoxy-phenoxy)pyrimidin-4-one (Example 179)

Using General Procedure 6 starting from EXAMPLE 136 and 4-methyl-2-(6-methyl-3-pyridyl)thiazole-5-carboxylic acid as reagents, EXAMPLE 179 was obtained. HRMS calculated for C₄₀H₄₀F₃N₇O₆S: 803.2713: found 804.2772 ([M+H]⁺ form).

### tert-butyl (3R,4R)-4-[(4S)-4-[[5-amino-4-(4-chloro-3-methoxy-phenoxy)-6-oxo-pyrimidin-1-yl]methyl]-3,3-difluoro-4-hydroxy-piperidine-1-carbonyl]-3-phenyl-piperidine-1-carboxylate (Example 180)

Using General Procedure 4 starting from Preparation R4m and Preparation 5b as reagents, the enantiomers were separated by chiral chromatography to give EXAMPLE 180. HRMS calculated for C₃₄H₄₀ClF₂N₅O₇: 703.2584: found 726.2469 ([M+Na]⁺ form).

### 5-amino-6-(4-chloro-3-methoxy-phenoxy)-3-[[(4S)-3,3-difluoro-4-hydroxy-1-[(3R,4R)-3-phenylpiperidine-4-carbonyl-4-piperidyl]methyl]pyrimidin-4-one hydrochloride (Example 181)

Using General Procedure 5 starting from EXAMPLE 180 as reagent, EXAMPLE 181 was obtained. HRMS calculated for C₂₉H₃₂ClF₂N₅O₅: 603.206; found 604.2127 ([M+H]⁺ form).

### 5-amino-6-(4-chloro-3-methoxy-phenoxy)-3-[[(4S)-3,3-difluoro-4-hydroxy-1-[(3R,4R)-1-[(2-methylpyrimidin-4-yl)methyl]-3-phenyl-piperidine-4-carbonyl-4-piperidyl]methyl]pyrimidin-4-one (Example 182)

Using General Procedure 9 starting from EXAMPLE 181 and 4-(chloromethyl)-2-methyl-pyrimidine as reagents. EXAMPLE 182 was obtained. HRMS calculated for C₃₅H₃₈ClF₂N₇O₅: 709.2591; found 710.2659 ([M+H]⁺ form).

### 5-amino-6-(4-chloro-3-methoxy-phenoxy)-3-[[(4S)-3,3-difluoro-4-hydroxy-1-[(3R,4R)-1-[(5-methylpyrazin-2-yl)methyl]-3-phenyl-piperidine-4-carbonyl]-4-piperidyl]methyl]pyrimidin-4-one (Example 183)

Using General Procedure 9 starting from EXAMPLE 181 and 2-(chloromethyl)-5-methyl-pyrazine hydrochloride as reagents, EXAMPLE 183 was obtained. HRMS calculated for C₃₅H₃₈ClF₂N₇O₅: 709.2591: found 710.2656 ([M+H]⁺ form).

### 5-amino-6-(4-chloro-3-methoxy-phenoxy)-3-[[(4S)-3,3-difluoro-4-hydroxy-1-[(3R,4R)-1-[4-methyl-2-(6-methyl-3-pyridyl)thiazole-5-carbonyl]-3-phenyl-piperidine-4-carbonyl]-4-piperidyl]methyl]pyrimidin-4-one (Example 184)

Using General Procedure 6 starting from EXAMPLE 181 and 4-methyl-2-(6-methyl-3-pyridyl)thiazole-5-carboxylic acid as reagents, EXAMPLE 184 was obtained. HRMS calculated for C₄₀H₄₀ClF₂N₇O₆S: 819.2418; found 820.2483 ([M+H]⁺ form).

### 5-amino-3-[[(4S)-3,3-difluoro-4-hydroxy-1-[(3R,4R)-1-[4-methyl-2-[6-methyl-5-(trifluoromethyl)-3-pyridyl]thiazole-5-carbonyl]-3-phenyl-piperidine-4-carbonyl]-4-piperidyl]methyl]-6-(4-fluorophenoxy)pyrimidin-4-one (Example 185)

Using General Procedure 11 starting from EXAMPLE 41 and 2-methyl-5-(4,4,5,5-tetramethyl-1,3,2-dioxaborolan-2-yl)-3-(trifluoromethyl)pyridine as reagents, EXAMPLE 185 was obtained. HRMS calculated for C₄₀H₃₇F₆N₇O₅S: 841.2481; found 842.2549 ([M+H]⁺ form).

### tert-butyl (3R,4R)-4-[(4S)-4-[[5-amino-4-(4-cyclopropyl-3-methoxy-phenoxy)-6-oxo-pyrimidin-1-yl]methyl]-3,3-difluoro-4-hydroxy-piperidine-1-carbonyl]-3-phenyl-piperidine-1-carboxylate (Example 186)

Using General Procedure 4 starting from Preparation R4n and Preparation R5b as reagents, the enantiomers were separated by chiral chromatography to give EXAMPLE 186. HRMS calculated for C₃₇H₄₅F₂N₅O₇: 709.3287; found 732.3172 ([M+Na]⁺ form).

### 5-amino-6-(4-cyclopropyl-3-methoxy-phenoxy)-3-[[(4S)-3,3-difluoro-4-hydroxy-1-[(3R,4R)-3-phenylpiperidine-4-carbonyl]-4-piperidyl]methyl]pyrimidin-4-one hydrochloride (Example 187)

Using General Procedure 5 starting from EXAMPLE 186 as reagent, EXAMPLE 187 was obtained. HRMS calculated for C₃₂H₃₇F₂N₅O₅: 609.2763; found 610.2834 ([M+H]⁺ form).

### 5-amino-6-(4-cyclopropyl-3-methoxy-phenoxy)-3-[[(4S)-3,3-difluoro-4-hydroxy-1-[(3R,4R)-1-[(2-methylpyrimidin-4-yl)methyl]-3-phenyl-piperidine-4-carbonyl]-4-piperidyl]methyl]pyrimidin-4-one (Example 188)

Using General Procedure 9 starting from EXAMPLE 187 and 4-(chloromethyl)-2-methyl-pyrimidine as reagents, EXAMPLE 188 was obtained. HRMS calculated for C₃₈H₄₃F₂N₇O₅: 715.3293; found 716.3353 (M+H]⁺ form).

### 5-amino-3-[[1-[(3R,4R)-1-[2-(3,3-difluorocyclobutyl)-4-methyl-thiazole-5-carbonyl 3-phenyl-piperidine-4-carbonyl]-4-hydroxy-4-piperidyl]methyl]-6-(4-fluorophenoxy)pyrimidin-4-one (Example 189)

Using General Procedure 6 starting from EXAMPLE 21 and 2-(3,3-difluorocyclobutyl)-4-methyl-thiazole-5-carboxylic acid as reagents, EXAMPLE 189 was obtained. FIRMS calculated for C₃₇H₃₉F₃N₆O₅S: 736.2655; found 737.2724 ([M+H]⁺ form).

### 5-amino-3-[[(4S)-1-[(3R,4R)-1-[2-(3,3-difluorocyclobutyl)-4-methyl-thiazole-5-carbonyl]-3-phenyl-piperidine-4-carbonyl]-3,3-difluoro-4-hydroxy-4-piperidyl]methyl-6-(4-fluorophenoxy)pyrimidin-4-one (Example 190)

Using General Procedure 6 starting from EXAMPLE 11 and 2-(3,3-difluorocyclobutyl)-4-methyl-thiazole-5-carboxylic acid as reagents, EXAMPLE 190 was obtained, HRMS calculated for C37H37F5N6O5S: 772.2466; found 773.2533 ([M+H]+ form).

### 5-amino-6-(4-fluorophenoxy)-3-[[4-hydroxy-1-[(3R,4R)-1-(4-methyl-2-tetrahydropyran-4-yl-thiazole-5-carbonyl)-3-phenyl-piperidine-4-carbonyl]-4-piperidyl]methyl]pyrimidin-4-one (Example 191)

Using General Procedure 6 starting from EXAMPLE 21 and 4-methyl-2-tetrahydropyran-4-yl-thiazole-5-carboxylic acid as reagents, EXAMPLE 191 was obtained. HRMS calculated for C₃₈H₄₃FN₆O₆S: 730.2949: found 731.3013 ([M+H]⁺ form).

### 5-amino-3-[[(4S)-3,3-difluoro-4-hydroxy-1-[(3R,4R)-1-(4-methyl-2-tetrahydropyran-4-yl-thiazole-5-carbonyl)-3-phenyl-piperidine-4-carbonyl]-4-piperidyl]methyl]-6-(4-fluorophenoxy)pyrimidin-4-one (Example 192)

Using General Procedure 6 starting from EXAMPLE 11 and 4-methyl-2-tetrahydropyran-4-yl-thiazole-5-carboxylic acid as reagents, EXAMPLE 192 was obtained. HRMS calculated for C₃₈H₄₁F₃N₆O₆S: 766.2761; found 767.2827 ([M+H]⁺ form).

### tert-butyl (3R,4R)-4-[(3SR,4RS)-4-[[5-amino-4-(4-fluorophenoxy)-6-oxo-pyrimidin-1-yl}methyl]-3-fluoro-4-hydroxy-piperidine-1-carbonyl]-3-phenyl-piperidine-1-carboxylate

### and

### tert-butyl (3R,4R)-4-[(3RS,4SR)-4-[[5-amino-4-(4-fluorophenoxy)-6-oxo-pyrimidin-1-yl]methyl]-3-fluoro-4-hydroxy-piperidine-1-carbonyl]-3-phenyl-piperidine-1-carboxylate (Example 193)

Using General Procedure 6 starting from Preparation R11b and (3R,4R)-1-(tert-butoxycarbonyl)-3-phenylpiperidine-4-carboxylic acid as reagents, EXAMPLE 193 was obtained. HRMS calculated for C₃₃H₃₉F₂N₅O₆: 639.2869; found 5402411 and 540.2424 ([M+H-C₄H₈-CO₂]⁺ form).

### tert-butyl (3R,4R)-4-[(3SR,4SR)-4-[[5-amino-4-(4-fluorophenoxy)-6-oxo-pyrimidin-1-yl]methyl]-3-fluoro-4-hydroxy-piperidine-1-carbonyl]-3-phenyl-piperidine-1-carboxylate

### and

### tert-butyl (3R,4R)-4-[(3RS,4RS)-4-[[5-amino-4-(4-fluorophenoxy)-6-oxo-pyrimidin-1-yl]methyl]-3-fluoro-4-hydroxy-piperidine-1-carbonyl]-3-phenyl-piperidine-1-carboxylate (Example 194)

Using General Procedure 6 starting from Preparation R11a and (3R,4R)-1-(tert-butoxycarbonyl)-3-phenylpiperidine-4-carboxylic acid as reagents, EXAMPLE 194 was obtained. HRMS calculated for C₃₃H₃₉F₂N₅O₆: 639,2869; found 540,2403 and 540.2401 ([M+H-C₄H₈-CO₂]⁺ form).

### 5-amino-3-[[(3SR,4RS)-3-fluoro-4-hydroxy-1-[(3R,4R)-3-phenylpiperidine-4-carbonyl]-4-piperidyl]methyl]-6-(4-fluorophenoxy)pyrimidin-4-one hydrochloride

### and

### 5-amino-3-[[(3RS,4SR)-3-fluoro-4-hydroxy-1-[(3R,4R)-3-phenylpiperidine-4-carbonyl]-4-piperidyl]methyl]-6-(4-fluorophenoxy)pyrimidin-4-one hydrochloride (Example 195)

Using General Procedure 5 starting from EXAMPLE 193 as reagent, EXAMPLE 195 was obtained. H-RMS calculated for C₂₈H₃₁F₂N₅O₄: 539.2344; found 540.2407 and 540.2410 ([M+H]⁺ form),

### 5-amino-3-[[(3SR,4SR)-3-fluoro-4-hydroxy-1-[(3R,4R)-3-phenylpiperidine-4-carbonyl]-4-piperidyl]methyl]-6-(4-fluorophenoxy)pyrimidin-4-one hydrochloride

### and

### 5-amino-3-[[(3RS,4RS)-3-fluoro-4-hydroxy-1-[(3R,4R)-3-phenylpiperidine-4-carbonyl]-4-piperidyl]methyl]-6-(4-fluorophenoxy)pyrimidin-4-one hydrochloride (Example 196)

Using General Procedure 5 starting from EXAMPLE 194 as reagent. EXAMPLE 196 was obtained. HRMS calculated for C₂₈H₃₁F₂N₅O₄: 539.2344: found 540.2399 and 540.2398 ([M+H]⁺ form).

### 5-amino-3-[[(3SR,4RS)-3-fluoro-4-hydroxy-1- (3R,4R)-1-[4-methyl-2-(6-methyl-3-pyridyl)thiazole-5-carbonyl]-3-phenyl-piperidine-4carbonyl]-4-piperidyl]methyl]-6-(4-fluorophenoxy)pyrimidin-4-one

### and

### 5-amino-3-[[(3RS,4SR)-3-fluoro-4-hydroxy-1-(3R,4R)-1-[4-methyl-2-(6-methyl-3-pyridyl)thiazole-5-carbonyl]-3-phenyl-piperidine-4-carbonyl]-4-piperidyl]methyl]-6-(4-fluorophenoxy)pyrimidin-4-one (Example 197)

Using General Procedure 6 starting from EXAMPLE 195 and 4-methyl-2-(6-methyl-3-pyridyl)thiazole-5-carboxylic acid as reagents, Example 197 was obtained. HRMS calculated for C₃₉H₃₉F₂N₇O₅S: 755.2701: found 756.2764 and 756.2767 (M+H]⁺ form).

### 5-amino-3-[(3SR,4SR)-3-fluoro-4-hydroxy-1-(3R,4R)-1-[4-methyl-2-(6-methyl-3-pyridyl)thiazole-5-carbonyl]-3-phenyl-piperidine-4-carbonyl]-4-piperidyl]methyl]-6-(4-fluorophenoxy)pyrimidin-4-one

### and

### 5-amino-3-[[(3RS,4RS)-3-fluoro-4-hydroxy-1-[(3R,4R)-1-[4-methyl-2-(6-methyl-3-pyridyl)thiazole-5-carbonyl]-3-phenyl-piperidine-4-carbonyl]-4-piperidyl]methyl]-6-(4-fluorophenoxy)pyrimidin-4-one (Example 198)

Using General Procedure 6 starting from EXAMPLE 196 and 4-methyl-2-(6-methyl-3-pyridyl)thiazole-5-carboxylic acid as reagents, EXAMPLE 198 was obtained. HRMS calculated for C₃₉H₃₉F₂N₇O₅S: 755.2701; found 756.2769 and 756.2759 ([M+H]⁺ form).

### 5-amino-6-(4-fluorophenoxy)-3-[[4-hydroxy-1-[(3R,4R)-1-[4-methyl-2-(1-methyl-3-piperidyl)thiazole-5′-carbonyl]-3-phenyl-piperidine-4-carbonyl]-4-piperidyl]methyl]pyrimidin-4-one (Example 199)

Using General Procedure 6 starting from EXAMPLE 21 and 4-methyl-2-(I-methyl-3-piperidyl)thiazole-5-carboxylic acid hydrochloride as reagents, EXAMPLE 199 was obtained. HRMS calculated for C₃₉H₄₆FN₇O₅S: 743.3265: found 744.3326 ([M+H]⁺ form).

### 5-amino-6-[4-(hydroxymethyl)phenoxyl-3-[[4-hydroxy-1-[(3R,4R)-1-[(2-methylpyrimidin-4-yl)methyl]-3-phenyl-piperidine-4-carbonyl]4-piperidyl]methyl]pyrimidin-4-one (Example 200)

Using General Procedure 4 starting from Preparation R4l and Preparation R5a as reagents, tert-butyl (3R,4R)-4-[4-[[5-amino-4-(4-formylphenoxy)-6-oxo-pyrimidin-1-yl]methyl]-4-hydroxy-piperidine-1-carbonyl]-3-phenyl-piperidine-1-carboxylate was obtained as a crude product.

Tert-butyl (3R,4R)-4-[4-[[5-amino-4-(4-formylphenoxy)-6-oxo-pyrimidin-1-yl]methyl-4-hydroxy-piperidine-1-carbonyl]-3-phenyl-piperidine-1-carboxylate was reacted using General procedure 5 to give 4-{5-amino-1-{14-hydroxy-1-[(3R,4R)-3-phenylpiperidine-4-carbonyl]-4-piperidyl]methyl]-6-oxo-pyrimidin-4-yl]oxybenzaldehyde hydrochloride, which was reacted with 2-(chloromethyl)-4-methyl-pyrimidine using General procedure 9 to give 4-[5-amino-1-[[4-hydroxy-1-[(3R,4R-1-[(2-methylpyrimidin-4-yl)methyl]-3-phenyl-piperidine-4-carbonyl]-4-piperidyl]methyl]-6-oxo-pyrimidin-4-yl]oxybenzaldehyde.

4-[5-amino-1-[[4-hydroxy-1-[(3R,4R)-1-[(2-methylpyrimidin-4-yl)methyl]-3-phenyl-piperidine-4-carbonyl]-4-piperidyl]methyl]-6-oxo-pyrimidin-4-yl]oxybenzaldehyde (24 mg, 0.037 mmol) and sodium borohydride (1.4 mg, 1 eq.) were dissolved in methanol (3 ml) at r.t. for 20 hours. The residue was directly injected to preparative LC (on C-18 Gemini-NX 5 μm column, 5 mM aqueous NH₄HCO₃-MeCN, gradient) and evaporated to give EXAMPLE 200. HRMS calculated for C₃₅H₄₁N₇O₅: 639,3169: found 640.3221 ([M+H]⁺ form).

### 5-amino-3-[(4S)-3,3-difluoro-4-hydroxy-1-[(3R,4R)-1-[(5-methylpyrazin-2-yl)methyl]-3-phenyl-piperidine-4-carbonyl]-4-piperidyl]methyl]-6-(4-fluorophenoxy)pyrimidin-4-one (Example 201)

Using General Procedure 9 starting from EXAMPLE 11 and 2-(chloromethyl)-5-methyl-pyrazine hydrochloride as reagents, EXAMPLE 201 was obtained. HRMS calculated for C₃₄H₃₆F₃N₇O₄: 663.2781: found 664.2846 ([M+H]⁺ form).

### 5-amino-3-[[(4S)-3,3-difluoro-4-hydroxy-1-[(3R,4R)-3-phenyl-1-(pyrazin-2-ylmethyl)piperidine-4-carbonyl]-4-piperidyl]methyl]-6-(4-fluorophenoxy)pyrimidin-4-one (Example 202)

Using General Procedure 9 starting from EXAMPLE 11 and 2-(chloromethyl)pyrazine hydrochloride as reagents, EXAMPLE 202 was obtained. HRMS calculated for C₃₃H₃₄F₃N₇O₄: 649.2625; found 650.2687 ([M+H]⁺ form).

### 5-amino-3-[[(4S)-3,3-difluoro-4-hydroxy-1-[(3R,4R)-3-phenyl-1-(pyridazin-3-ylmethyl)piperidine-4-carbonyl-4-piperidyl]methyl]-6-(4-fluorophenoxy)pyrimidin-4-one (Example 203)

Using General Procedure 9 starting from EXAMPLE 11 and 3-(bromomethyl)pyridazine hydrobromide as reagents, EXAMPLE 203 was obtained. HRMS calculated for C₃₃H₃₄F₃N₇O₄: 649.2625: found 650.2687 ([M+H]⁺ form).

5-amino-3-[[(4S)-3,3-difluoro-4-hydroxy-1-(3R,4R)-3-phenyl-1-(pyrimidin-2-ylmethyl)piperidine-4-carbonyl]-4-piperidyl]methyl]-6-(4-fluorophenoxy)pyrimidin-4-one (Example 204)

Using General Procedure 9 starting from EXAMPLE 11 and 2-(chloromethyl)pyrimidine hydrochloride as reagents. EXAMPLE 204 was obtained. HRMS calculated for C₃₃H₃₄F₃N₇O₄: 649,2625; found 650.2706 ([M+H]⁺ form).

### 5-amino-3-[[(4S)-3,3-difluoro-4-hydroxy-1-[(3R,4R)-1-[(4-methylpyrimidin-2-yl)methyl]-3-phenyl-piperidine-4-carbonyl-4-piperidyl]methyl]-6-(4-fluorophenoxy)pyrimidin-4-one (Example 205)

Using General Procedure 9 starting from EXAMPLE 11 and 2-(chloromethyl)-4-methyl-pyrimidine as reagents, EXAMPLE 205 was obtained. HRMS calculated for C₃₄H₃₆F₃N₇O₄: 663.2781; found 664.2844 ([M+H]⁺ form).

### 5-amino-3-[[(4S)-1-[(3R,4R)-1-[(4,6-dimethylpyrimidin-2-yl)methyl]-3-phenyl-piperidine-4-carbonyl]-3,3-difluoro-4-hydroxy-4-piperidyl]methyl]-6(4-fluorophenoxy)pyrimidin-4-one (Example 206)

Using General Procedure 9 starting from EXAMPLE 11 and 2-(chloromethyl)-4,6-dimethyl-pyrimidine as reagents, EXAMPLE 206 was obtained. HRMS calculated for C₃₅H₃₈F₃N₇O₄: 677.2938; found 678.302 ([M+H]⁺ form).

### 5-amino-3-[[(4S)-3,3-difluoro-4-hydroxy-1-[(3R,4R)-1-[(6-methylpyrazin-2-yl)methyl]-3-phenyl-piperidine-4-carbonyl]-4-piperidyl]methyl]-6-(4-fluorophenoxy)pyrimidin-4-one (Example 207)

Using General Procedure 9 starting from EXAMPLE 11 and 2-(chloromethyl)-6-methyl-pyrazine hydrochloride as reagents, EXAMPLE 207 was obtained. HRMS calculated for C₃₄H₃₆F₃N₇O₄: 663.2781; found 664.2874 ([M+H]⁺ form).

### tert-butyl (3R,4R)-4-[(4S)-4-[[5-amino-4-(4-cyanophenoxy)-6-oxo-pyrimidin-1-yl]methyl]-3,3-difluoro-4-hydroxy-piperidine-1-carbonyl]-3-phenyl-piperidine-1-carboxylate (Example 208)

Using General Procedure 4 starting from Preparation R4j and Preparation R5b as reagents, the enantiomers were separated by chiral chromatography to give EXAMPLE 208. HRMS calculated for C₃₄H₃₈F₂N₆O₆: 664.2821; found 687.2704 ([M+Na]⁺ form).

### 4-[5-amino-1-[[(4S)-3,3-difluoro-4-hydroxy-1-(3R,4R)-3-phenylpiperidine-4-carbonyl]-4-piperidyl]methyl]-6-oxo-pyrimidin-4-yl]oxybenzonitrile hydrochloride (Example 209)

Using General Procedure 5 starting from EXAMPLE 208 as reagent, EXAMPLE 209 was obtained. HRMS calculated for C₂₉H₃₀F₂N₆O₄: 564.2297; found 565.2355 ([M+H]⁺ form).

### 5-amino-6-[4-(aminomethyl)phenoxyl-3-[(4S)-3,3-difluoro-4-hydroxy-1-[(3R,4R)-1-14-methyl-2-(6-methyl-3-pyridyl)thiazole-5-carbonyl]-3-phenyl-piperidine-4-carbonyl]-4-piperidyl]methyl]pyrimidin-4-one (Example 210)

Using General Procedure 6 starting from EXAMPLE 209 and 4-methyl-2-(6-methyl-3-pyridyl)thiazole-5-carboxylic acid as reagents, 4-[5-amino-1-[[(4S)-3,3-difluoro-4-hydroxy-1-[(3R,4R)-1-[4-methyl-2-(6-methyl-3-pyridyl)thiazole-5-carbonyl]-3-phenyl-piperidine-4-carbonyl]-4-piperidyl]methyl]-6-oxo-pyrimidin-4-yl]oxybenzonitrile was obtained as a crude product and was directly reacted using General Procedure 2 to give EXAMPLE 210. HRMS calculated for C₄₀H₄₂F₂N₅O₅S: 784.2967; found 785,3033 ([M+H]⁺ form).

### 5-amino-3-[[(4S)-3,3-difluoro-4-hydroxy-1-[(3R,4R)-1-[(3-methylpyrazin-2-yl)methyl]-3-phenyl-piperidine-4-carbonyl]-4-piperidyl]methyl]-4(4-fluorophenoxy)pyrimidin-4-one (Example 211)

Using General Procedure 9 starting from EXAMPLE 11 and 2-(chloromethyl)-3-methyl-pyrazine hydrochloride as reagents, EXAMPLE 211 was obtained. HRMS calculated for C₃₄H₃₆F₃N₇O₄: 663.2781; found 664.2847 ([M+H]⁺ form).

### 5-amino-3-[[(4S)-3,3-difluoro-4-hydroxy-1-[(3R,4R)-3-phenyl-1-(pyrimidin-4-ylmethyl)piperidine-4-carbonyl]-4-piperidyl]methyl]-6-(4-fluorophenoxy)pyrimidin-4-one (Example 212)

Using General Procedure 9 starting from EXAMPLE 11 and 4-(chloromethyl)pyrimidine as reagents, EXAMPLE 212 was obtained. HRMS calculated for C₃₃H₃₄F₃N₇O₄: 649.2625; found 650.2672 ([M+H]⁺ form).

### 5-amino-3-[[(4S)-3,3-difluoro-4-hydroxy-1-[(3R,4R)-3-phenyl-1-(pyrazin-2-ylmethyl)piperidine-4-carbonyl]-4-piperidyl]methyl]-6-(4-fluoro-3-methoxy-phenoxy)pyrimidin-4-one (Example 213)

Using General Procedure 9 starting from EXAMPLE 136 and 2-(chloromethyl)pyrazine hydrochloride as reagents, EXAMPLE 213 was obtained. HRMS calculated for C₃₄H₃₆N₇O₅: 679.273; found 680.2784 ([M+H]⁺ form).

### 5-amino-3-[[(4S)-3,3-difluoro-4-hydroxy-1-[(3R,4R)-3-phenyl-1-(pyridazin-3-ylmethyl)piperidine-4-carbonyl-4-piperidyl]methyl]6-(4-fluoro-3-methoxy-phenoxy)pyrimidin-4-one (Example 214)

Using General Procedure 9 starting from EXAMPLE 136 and 3-(bromomethyl)pyridazine hydrobromide as reagents, EXAMPLE 214 was obtained. HRMS calculated for C₃₄H₃₆F₃N₇O₅: 679.273; found 680.2781 ([M+H]⁺ form)

### 5-amino-3-[[(4S)-3,3-difluoro-4-hydroxy-1-[(3R,4R)-3-phenyl-1-(pyrimidin-2-ylmethyl)piperidine-4-carbonyl]-4-piperidyl]methyl]-6-(4-fluoro-3-methoxy-phenoxy)pyrimidin-4-one (Example 215)

Using General Procedure 9 starting from EXAMPLE 136 and 2-(chloromethyl)pyrimidine hydrochloride as reagents, EXAMPLE 215 was obtained. FIRMS calculated for C₃₄H₃₆F₃N₇O₅: 679.273; found 680.2786 ([M+H]⁺ form).

### 5-amino-3-[[(4S)-3,3-difluoro-4-hydroxy-1-[(3R,4R)-1-[(4-methylpyrimidin-2-yl)methyl]-3-phenyl-piperidine-4-carbonyl]-4-piperidyl]methyl-6-(4-fluoro-3-methoxy-phenoxy)pyrimidin-4-one (Example 216)

Using General Procedure 9 starting from EXAMPLE 136 and 2-(chloromethyl)-4-methyl-pyrimidine as reagents, EXAMPLE 216 was obtained. HRMS calculated for C₃₅H₃₈F₃N₇O₅: 693.2886; found 694.2953 ([M+H]⁺ form).

### 5-amino-3-[[(4S)-1-[(3R,4R)-1-[(4,6-dimethylpyrimidin-2-yl)methyl]-3-phenyl-piperidine-4-carbonyl-3,3-difluoro-4-hydroxy-4-piperidyl]methyl]-6-(4-fluoro-3-methoxy-phenoxy)pyrimidin-4-one (Example 217)

Using General Procedure 9 starting from EXAMPLE 136 and 2-(chloromethyl)-4,6-dimethyl-pyrimidine as reagents. EXAMPLE 217 was obtained. HRMS calculated for C₃₆H₄₀F₃N₇O₅: 707,3043; found 708.3094 ([M+H]⁺ form)

### 5-amino-3-[[(4S)-3,3-difluoro-4-hydroxy-1-[(3R,4R)-1-[(6-methylpyrazin-2-yl)methyl]-3-phenyl-piperidine-4-carbonyl]-4-piperidyl]methyl]-6-(4-fluoro-3-methoxy-phenoxy)pyrimidin-4-one (Example 218)

Using General Procedure 9 starting from EXAMPLE 136 and 2-(chloromethyl)-O-methyl-pyrazine hydrochloride as reagents, EXAMPLE 218 was obtained. HRMS calculated for C₃₅H₃₈F₃N₇O₅:693.2886; found 694.2942 ([M+H]⁺ form).

### 5-amino-3-[[(4S)-3,3-difluoro-4-hydroxy-1-[(3R,4R)-1-[(3-methylpyrazin-2-yl)methyl]-3-phenyl-piperidine-4-carbonyl]-4-piperidyl]methyl]-6-(4-fluoro-3-methoxy-phenoxy)pyrimidin-4-one (Example 219)

Using General Procedure 9 starting from EXAMPLE 136 and 2-(chloromethyl)-3-methyl-pyrazine; hydrochloride as reagents, EXAMPLE 219 was obtained. HRMS calculated for C₃₅H₃₈F₃N₇O₅: 693.2886: found 694.2938 ([M+H]⁺ form),

### 5-amino-3-[[(4S)-3,3-difluoro-4-hydroxy-1-[(3R,4R)-3-phenyl-1-(pyrimidin-4-ylmethyl)piperidine-4-carbonyl]-4-piperidyl]methyl]-6-(4-fluoro-3-methoxy-phenoxy)pyrimidin-4-one (Example 220)

Using General Procedure 9 starting from EXAMPLE 136 and 4-(chloromethyl)pyrimidine as reagents, EXAMPLE 220 was obtained. HRMS calculated for C₃₄H₃₆F₃N₇O₅: 679.273; found 680.2795 ([M+H]⁺ form)

### 5-amino-6-(4-chlorophenoxy)-3-[[(4S)-3,3-difluoro-4-hydroxy-1-[(3R,4R)-1-[(5-methylpyrazin-2-yl)methyl]-3-phenyl-piperidine-4-carbonyl]-4-piperidyl]methyl]pyrimidin-4-one (Example 221)

Using General Procedure 9 starting from EXAMPLE 167 and 2-(chloromethyl)-5-methyl-pyrazine hydrochloride as reagents, EXAMPLE 221 was obtained. HRMS calculated for C₃₄H₃₆ClF₂N₇O₄: 679.2485; found 680.2539 ([M+H]⁺ form).

### 5-amino-6-[4-(aminomethyl)phenoxy]-3-[[(4S)-1-[(3R,4R)-1-(3,5-dichlorobenzoyl)-3-phenyl-piperidine-4-carbonyl]-3,3-difluoro-4-hydroxy-4-piperidyl]methyl]pyrimidin-4-one (Example 222)

Using General Procedure 6 starting from EXAMPLE 209 and 3,5-dichlorobenzoic acid as reagents, 4-[5-amino-1-[[(4S)-1-[(3R,4R)-1-(3,5-dichlorobenzoyl)-3-phenyl-piperidine-4-carbonyl]-3,3-difluoro-4-hydroxy-4-piperidyl]methyl]-6-oxo-pyrimidin-4-yl]oxybenzonitrile was obtained as a crude product and was directly reacted using General Procedure 2 to give EXAMPLE 222. HRMS calculated for C₃₆H₃₆Cl₂F₂N₆O₅: 740.2092; found 741.2176 ([M+H]⁺ form).

### tert-butyl N-[[4-[5-amino-1-[[4-hydroxy-1-[(3R,4R)-1-[4-methyl-2-(6-methyl-3-pyridyl)thiazole-5-carbonyl]-3-phenyl-piperidine-4-carbonyl]-4-piperidyl]methyl]-6-oxo-pyrimidin-4-yl]oxyphenyl]methyl]carbamate (Example 223)

EXAMPLE 210 (1.0 eq.), tert-butoxycarbonyl tert-butyl carbonate (3.8 eq.), and sodium hydrogen carbonate (5.0 eq.) were dissolved in THF and water (1:1). The reaction mixture was stirred at r.t. till completion. The reaction mixture was extracted with EtOAc. The combined organic phase dried on MgSO₄ and the solvent was evaporated. The crude product was purified by preparative LC (on C-18 Gemini-NX 5 μm column, 5 mM aqueous NH₄HCO₃-MeCN, gradient). Solvent was evaporated under reduced pressure to give EXAMPLE 223. HRMS calculated for C₄₅H₄₂N₈O₇S: 848.368; found 849.3732 ([M+H]+ form).

### 5-amino-6-[4-(aminomethyl)phenoxy]-3-[[4-hydroxy-1-(3R,4R)-1-[2-(6-methyl-3-pyridyl)thiazol-5-yl]methyl]-3-phenyl-piperidine-4-carbonyl]-4-piperidyl]methyl]pyrimidin-4-one (Example 224)

Using General Procedure 4 starting from Preparation R4j and Preparation R8a as reagents, tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate was obtained.

Using General Procedure 5 starting from tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[[4-hydroxy-1-[(3R,4R)-3-phenylpiperidine-4-carbonyl]-4-piperidyl]methyl-6-oxo-pyrimidin-4-yl]oxybenzonitrile.

Using General Procedure 9 starting from 4-[5-amino-1-[[4-hydroxy-1-(3R,4R)-3-phenylpiperidine-4-carbonyl]-4-piperidyl]methyl]-6-oxo-pyrimidin-4-yl]oxybenzonitrile and 5-[15-(chloromethyl)-1,3-thiazol-2-yl]-2-methylpyridine as reagents, 4-[5-amino-1-[[4-hydroxy-1-[(3R,4R)-1-[[2-(6-methyl-3-pyridyl)thiazol-5-yl]methyl]-3-phenyl-piperidine-4-carbonyl]-4-piperidyl]methyl]-6-oxo-pyrimidin-4-yl]oxybenzonitrile was obtained.

Using General Procedure 2 starting from 4-[5-amino-1-[[4-hydroxy-1-[(3R,4R)-1-[[2-(6-methyl-3-pyridyl)thiazol-5-yl]methyl]-3-phenyl-piperidine-4-carbonyl]-4-piperidyl]methyl]-6-oxo-pyrimidin-4-yl]oxybenzonitrile as reagent. EXAMPLE 224 was obtained. HRMS calculated for C₃₉H₄₄N₈O₄S: 720.3206: found 721.3275 ((M+H)⁺ form).

### 5-amino-6-[4-(aminomethyl)phenoxyl-3-[[4-hydroxy-1-[(3R,4R)-3-phenyl-1-(pyridine-3-carbonyl)piperidine-4-carbonyl]-4-piperidyl]methyl]pyrimidin-4-one (Example 225)

### and

### 5-amino-6-[4-(aminomethyl)phenoxy]-3-[[1-[(3R,4R)-1-(5-chloropyridine-3-carbonyl)-3-phenyl-piperidine-4-carbonyl]-4-hydroxy-4-piperidyl[methyl]pyrimidin-4-one (Example 226)

Using General Procedure 4 starting from Preparation R4j and Preparation R5a as reagents, tert-butyl (3R,4R)-4-[4-{5-amino-4-(4-cyanophenoxy)-6-oxo-pyrimidin-1-yl]methyl]-4-hydroxy-piperidine-1-carbonyl]-3-phenyl-piperidine-1-carboxylate was obtained.

Using General Procedure 5 starting from tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile.

Using General Procedure 6 starting from 4-[5-amino-1-[[4-hydroxy-1-[(3R,4R)-3-phenylpiperidine-4-carbonyl]-4-piperidyl]methyl]-6-oxo-pyrimidin-4-yl]oxybenronitrile and 5-chloropyridine-3-carboxylic acid as reagents, 4-[5-amino-1-[[1-[(3R,4R)-1-(5-chloropyridine-3-carbonyl)-3-phenyl-piperidine-4-carbonyl]-4-hydroxy-4-piperidyl]methyl]-6-oxo-pyrimidin-4-yl]oxybenzonitrile was obtained.

Using General Procedure 2 starting from 4-[5-amino-1-[[1-[(3R,4R)-1-(5-chloropyridine-3-carbonyl)-3-phenyl-piperidine-4-carbonyl]-4-hydroxy-4-piperidyl]methyl]-6-oxo-pyrimidin-4-yl]oxybenzonitrile as reagent, EXAMPLE 225 and EXAMPLE 226 was obtained.

EXAMPLE 225: FIRMS calculated for C₃₅H₃₉N₇O₅: 637.3013; found 638.3085 ((M+H)⁺ form).

EXAMPLE 226: HRMS calculated for C₃₅H₃₈ClN₇O₅: 671.2623; found 672.2701 ((M+H)⁺ form).

### 5-amino-6-[4-(aminomethyl)phenoxy]-3-[[4-hydroxy-1-[(3R,4R)-1-[4-methyl-2-[6-(trifluoromethyl)-3-pyridyl]thiazole-5-carbonyl]-3-phenyl-piperidine-4-carbonyl]-4-piperidyl]methyl]pyrimidin-4-one (Example 227)

Using General Procedure 4 starting from Preparation R4j and Preparation R8a as reagents, tert-butyl (3R,4R)-4-[4-[[5-amino-4-(4-cyanophenoxy)-6-oxo-pyrimidin-1-yl}methyl]-4-hydroxy-piperidine-1-carbonyl]-3-phenyl-piperidine-1-carboxylate was obtained.

Using General Procedure 5 starting from tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[{4-hydroxy-1-((3R,4R)-3-phenylpiperidine-4-carbonyl]-4-piperidyl]methyl]-6-oxo-pyrimidin-4-yl]oxybenzonitrile.

Using General Procedure 6 starting from 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile and 2-bromo-4-methyl-thiazole-5-carboxylic acid as reagents, 4-[5-amino-1-[[1-[(3R,4R)-1-(2-bromo-4-methyl-thiazole-5-carbonyl)-3-phenyl-piperidine-4-carbonyl-4-hydroxy-4-piperidyl]methyl]-6-oxo-pyrimidin-4-yl]oxybenzonitrile was obtained.

Using General Procedure 11 starting from 4-[5-amino-1-[[1-[(3R,4R)-1-(2-bromo-4-methyl-thiazole-5-carbonyl)-3-phenyl-piperidine-4-carbonyl-4-hydroxy-4-piperidyl]methyl]-6-oxo-pyrimidin-4-yl]oxybenronitrile and [6-(trifluoromethyl)-3-pyridyl]boronic acid as reagent, 4-[5-amino-1-[[4-hydroxy-1-[(3R,4R)-1-[4-methyl-2-[6-(trifluoromethyl)-3-pyridyl]thiazole-5-carbonyl]-3-phenyl-piperidine-4-carbonyl]-4-piperidyl]methyl]-6-oxo-pyrimidin-4-yl]oxybenzonitrile was obtained.

Using General Procedure 2 starting from 4-[5-amino-1-[[4-hydroxy-1-[(3R,4R)-1-[4-methyl-2-[6-(trifluoromethyl)-3-pyridyl]thiazole-5-carbonyl]-3-phenyl-piperidine-4-carbonyl]-4-piperidyl]methyl]-6-oxo-pyrimidin-4-yl]oxybenzonitrile as reagent, EXAMPLE 227 was obtained. HRMS calculated for C₄₀H₄₁F₃N₅O₅S: 802.2873; found 803.2939 ((M+H)⁺ form).

### 5-amino-6-[4-(aminomethyl)phenoxyl-3-[[1-[(3R,4R)-1-[2-[6-(dimethylamino)-3-pyridyl]4-methyl-thiazole-5-carbonyl]-3-phenyl-piperidine-4-carbonyl}-4-hydroxy-4-piperidyl]methyl]pyrimidin-4-one (Example 228)

Using General Procedure 4 starting from Preparation R4j and Preparation R5a as reagents, tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate was obtained.

Using General Procedure 5 starting from tert-butyl (3R,4R)-4-[4-[[5-amino-4-(4-cyanophenoxy)-6-oxo-pyrimidin-1-yl]methyl 14-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[[4-hydroxy-1-[(3R,4R)-3-phentylpiperidine-4-carbonyl]-4-piperidyl]methyl]-6-oxo-pyrimidin-4-yl]oxybenzonitrile,

Using General Procedure 6 starting from 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile and 2-bromo-4-methyl-thiazole-5-carboxylic acid as reagents, 4-[5-amino-1-[[1-[(3R,4R)-1-(2-bromo-4-methyl-thiazole-5-carbonyl)-3-phenyl-piperidine-4-carbonyl]-4-hydroxy-4-piperidyl]methyl]-6-oxo-pyrimidin-4-yl]oxybenzonitrile was obtained.

Using General Procedure 11 starting from 4-[5-amino-1-[[1-[(3R,4R)-1-(2-bromo-4-methyl-thiazole-5-carbonyl)-3-phenyl-piperidine-4-carbonyl]-4-hydroxy-4-piperidyl]methyl]-6-oxo-pyrimidin-4-yl]oxybenzonitrile and 16-(dimethylamino)-3-pyridyl]boronic acid as reagent, 4-[5-amino-1-[11-[(3R,4R)-1-[2-[6-(dimethylamino)-3-pyridyl]-4-methyl-thiazole-5-carbonyl-3-phenyl-piperidine-4-carbonyl)-4-hydroxy-4-piperidyl]methyl]-6-oxo-pyrimidin-4-yl]oxybenzonitrile was obtained.

Using General Procedure 2 starting from 4-[5-amino-1-[[1-[(3R,4R)-1-12-[16-(dimethylamino)-3-pyridyl 14-methyl-thiazole-5-carbonyl-3-phenyl-piperidine-4-carbonyl}-4-hydroxy-4-piperidyl]methyl]-6-oxo-pyrimidin-4-yl]oxybenzonitrile as reagent, EXAMPLE 228 was obtained. HRMS calculated for C₄₁H₄₇N₉O₅S: 777.3421; found 778.3506 ((M+H)⁺ form).

### 5-amino-6-[4-(aminomethyl)phenoxy-3-[[4-hydroxy-1-[(3R,4R)-1-[5-(6-methyl-pyridyl)pyridine-3-carbonyl]-3-phenyl-piperidine-4-carbonyl]-4-piperidyl]methyl]pyrimidin-4-one (Example 229)

Using General Procedure 4 starting from Preparation R4j and Preparation R5a as reagents, tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate was obtained.

Using General Procedure 5 starting from tert-butyl (3R,4R)-4-[4-[5-amino-4-(4-cyanophenoxy)-6-oxo-pyrimidin-1-yl]methyl]-4-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[[4-hydroxy-1-[(3R,4R)-3-phenylpiperidine-4-carbonyl]-4-piperidyl]methyl 1-6-oxo-pyrimidin-4-yl]oxybenzonitrile.

Using General Procedure 6 starting from 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile and 5-chloropyridine-3-carboxylic acid as reagents, 4-[5-amino-1-[[1-[(3R,4R)-1-(5-chloropyridine-3-carbonyl)-3-phenyl-piperidine-4-carbonyl]-4-hydroxy-4-piperidyl]methyl]-6-oxo-pyrimidin-4-yl]oxybenzonitrile was obtained.

Using General Procedure 11 starting from 4-[5-amino-1-[[1-(3R,4R)-1-(5-chloropyridine-3-carbonyl)-3-phenyl-piperidine-4-carbonyl]-4-hydroxy-4-piperidyl]methyl]-6-oxo-pyrimidin-4-yl]oxybenzonitrile and (6-methyl-3-pyridyl)boronic acid as reagent, 4-[5-amino-1-[[4-hydroxy-1-[(3R,4R)-1-[5-(6-methyl-3-pyridyl)pyridine-3-carbonyl]-3-phenyl-piperidine-4-carbonyl]-4-piperidyl]methyl]-6-oxo-pyrimidin-4-yl]oxybenzonitrile was obtained.

Using General Procedure 2 starting from 4-[5-amino-1-[[4-hydroxy-1-[(3R,4R)-1-[5-(6-methyl-3-pyridyl)pyridine-3-carbonyl]-3-phenyl-piperidine-4-carbonyl]-4-piperidyl]methyl]-6-oxo-pyrimidin-4-yl]oxybenzonitrile as reagent, EXAMPLE 229 was obtained. HRMS calculated for C₄₁H₄₄N₅O₅: 728.3434; found 729.3489 ((M+H)⁺ form).

### 5-amino-6-[4-(aminomethyl)phenoxy]-3-[[4-hydroxy-1-[(3R,4R)-1-(isoquinoline-5-carbonyl)-3-phenyl-piperidine-4-carbonyl]-4-piperidyl]methyl]pyrimidin-4-one (Example 230)

Using General Procedure 4 starting from Preparation R41 and Preparation R5a as reagents, tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate was obtained.

Using General Procedure 5 starting from tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile.

Using General Procedure 6 starting from 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile and isoquinoline-5-carboxylic acid as reagents, 4-[5-amino-1-[[4-hydroxy-1-[(3R,4R)-1-(isoquinoline-5-carbonyl)-3-phenyl-piperidine-4-carbonyl]-4-piperidyl]methyl]-6-oxo-pyrimidin-4-yl]oxybenronitrile was obtained.

Using General Procedure 2 starting from 4-[5-amino-1-[[4-hydroxy-1-[(3R,4R)-1-(isoquinoline-5-carbonyl)-3-phenyl-piperidine-4-carbonyl]-4-piperidyl]methyl]-6-oxo-pyrimidin-4-yl]oxybenzonitrile as reagent, EXAMPLE 230 was obtained. HRMS calculated for C₃₉H₄₁N₇O₅: 687.3169; found 688.3237 ((M+H)⁻ form).

### 5-amino-6-[4-(aminomethyl)phenoxy]-3-[[4-hydroxy-1-[(3R,4R)-1-[[2-(6-methyl-3-pyridyl)pyrimidin-4-yl]methyl]-3-phenyl-piperidine-4-carbonyl]-4-piperidyl]methyl]pyrimidin-4-one (Example 231)

### Step 1: 2-(6-methyl-3-pyridyl)pyrimidine-4-carbaldehyde

2-chloropyrimidine-4-carbaldehyde (17.7 g, 124 mmol), (6-methyl-3-pyridyl)boronic acid (34 g, 248 mmol, 2 eq.), bis(di-tert-butyl(4-dimethylaminophenyl)phosphine) dichloropalladium(1l) (1.76 g, 0.02 eq, 2.48 mmol), and cesiumcarbonate (80.9 g, 2 eq., 248 mmol, 100 mass %) were dissolved in THF (85 mL) and water (85 mL). After stirring at 100° C. for until completion, the phases were separated and the aqueous phase extracted with EtOAc. The aqueous phase was set to pH=7 with 1M HCl, then extracted with DCM. The organic phases were combined and evaporated. The crude product was dissolved in 500 mL DCM, then 105 g Celite was added and the volatiles were removed under reduced pressure. Then it was purified via flash chromatography using DCM and EtOAc as eluents, gradient method 0-50% to give 2-(6-methyl-3-pyridyl)pyrimidine-4-carbaldehyde. HRMS calculated for C₁₁H₉N₃O: 199.0746; found 200.0813 ((M+H)⁺ form).

¹H-NMR (400 MHz, dmso-d6) δ ppm 10.03 (s, 1H), 9.46 (d, 1H), 9.23 (d, 1H), 8.63 (dd, 1H), 7.83 (d, 1H), 7.47 (d, 1H), 2.58 (s, 3H)

### Step 2: Example 231

Using General Procedure 4 starting from Preparation R4j and Preparation R5a as reagents, tert-butyl (3R,4R)-4-[4-[[5-amino-4-(4-cyanophenoxy)-6-oxo-pyrimidin-1-yl]methyl]-4-hydroxy-piperidine-1-carbonyl]-3-phenyl-piperidine-1-carboxylate was obtained.

Using General Procedure 5 starting from tert-butyl (3R,4R)-4-[4-[[5-amino-4-(4-cyanophenoxy)-6-oxo-pyrimidin-1-yl]methyl}-4-hydroxy-piperidine-1-carbonyl]-3-phenyl-piperidine-1-carboxylate as reagent, the crude product was purified by preparative LC (on C-18 Luna 10 μm column, 5 mM aqueous NH₄HCO₃-MeCN, gradient). Solvent was evaporated under reduced pressure to give 4-[5-amino-1-[[4-hydroxy-1-[(3R,4R)-3-phenylpiperidine-4-carbonyl]-4-piperidyl]methyl]-6-oxo-pyrimidin-4-yl]oxybenzonitrile.

Using General Procedure 10 starting from 4-[5-amino-1-[[4-hydroxy-1-[(3R,4R)-3-phenylpiperidine-4-carbonyl]-4-piperidyl]methyl-[6-oxo-pyrimidin-4-yl]oxybenronitrile and 2-(6-methyl-3-pyridyl)pyrimidine-4-carbaldehyde (as obtained in Step 1 above) as reagents, 4-[5-amino-1-[[4-hydroxy-1-[(3R,4R)-1-[[2-(6-methyl-3-pyridyl)pyrimidin-4-yl]methyl]-3-phenyl-piperidine-4-carbonyl]-4-piperidyl]methyl]-6-oxo-pyrimidin-4-yl]oxybenzonitrile was obtained.

Using General Procedure 2 starting from 4-[5-amino-1-[[4-hydroxy-1-[(3R,4R)-1-[[2-(6-methyl-3-pyridyl)pyrimidin-4-yl]methyl]-3-phenyl-piperidine-4-carbonyl]-4-piperidyl]methyl]-6-oxo-pyrimidin-4-yl]oxybenronitrile as reagent, EXAMPLE 231 was obtained. HRMS calculated for C₄₀H₄₅N₉O₄: 715.3594: found 716.3677 ((M+H)⁺ form)

### Pharmacological Study

### Example A: Evaluation of the Inhibition of USP7 by the Fluorescence Intensity (FLINT) Readings

USP7 activity was measured using Rhodamine-110 c-terminal labelled Ubiquitin as a substrate (UbiQ Bio), Incubation with USP7 results in the release of Rhodamine-110 leading to an increase in fluorescence which can be used in the continuous measurement of USP7 activity.

The USP7 reactions were performed in a 50 μL volume, in 384 well black solid low binding plates (Corning #3575). The reaction buffer consisted of 100 mM Bicine pH 8.0, 0.01% TritonX 100, 1 mM TCEP, and 10% DMSO.

0.25 nM His-His-USP7 (aa208-560, |C315A|) was incubated with compound (final concentration 10% DMSO) for 60 minutes at 30° C. The reaction was then initiated by the addition of 50) nM Ubiquitin-Rhodamine-110 substrate or 4 μM Ubiquitin-Rhodamine-110 substrate and the plate read every 3 minutes for 21 minutes to measure the release of Rhodamine-110. Fluorescence Intensity (FLINT) readings were measured using a Biomek Neo plate reader (Ex.485 nm, Em.535 nm).

The inhibition of increasing doses of compound was expressed as a percentage reduction in kinetic rate compared to the kinetic rates established between ‘DMSO only’ and ‘total inhibition’ controls (no USP7). The inhibitory concentrations that gave a 50% reduction in kinetic rate (IC₅₀) were determined, from 11-point dose response curves, in XL-Fit using a 4-Parameter Logistic Model 205 (Sigmoidal Dose-Response Model). The K, values were determined from the IC₅₀ values according to Cer et al. Nucleic Acids Res. 2009, July 1; 37 (WebServer issue): W441-W445.

The results presented in Table 1 below show that compounds of the invention inhibit interaction between USP7 protein and the fluorescent peptide described hereinbefore.

### Example B: In Vitro Cytotoxicity

The cytotoxicity studies were evaluated by MTT [3-(4,5-dimethylthiaxol-2-yl)-2,5-diphenyltetrazolium bromide|assay and carried out on Z138 mantle cell lymphoma tumour cell lines. The cells are distributed onto microplates and exposed to the test compounds for 96 hours. MTT is then added for 4 hours and converted by NAD(P)H-dependent cellular oxidoreductase enzymes in forma/an, which has a purple color. The viable cell number is proportional to the production of formazan salts and cell viability can be quantified by the absorbance of the solution at 540 nm with a spectrophotometer (Carmichael et al., Cancer Res. 1987, 47, 936-942). The results are expressed in IC₅₀ (the concentration of compound that inhibits cell viability by 50% compared to DMSO treated cells only) and are presented in Table 1 below.

The results show that the compounds of the invention are cytotoxic.

TABLE 1
IC₅₀ of USP7 inhibition and of cytotoxicity for Z138 cells

| EXAMPLE | Ki (M) USP7 FLINT | IC₅₀ (M) MTT |
|---|---|---|
| 1 | 1.03E−07 | NT |
| 2 | 1.91E−07 | NT |
| 3 | 1.25E−07 | NT |
| 4 | 1.15E−07 | NT |
| 5 | 2.99E−07 | NT |
| 6 | 3.98E−07 | NT |
| 7 | 1.37E−07 | NT |
| 8 | 1.31E−07 | NT |
| 9 | 8.25E−08 | NT |
| 10 | 1.04E−07 | NT |
| 11 | 9.94E−08 | 3.9E−07 |
| 13 | 2.33E−08 | 1.06E−08 |
| 14 | 1.15E−07 | NT |
| 15 | 2.86E−07 | NT |
| 16 | 2.32E−07 | NT |
| 17 | 1.16E−07 | NT |
| 18 | 3.29E−07 | NT |
| 19 | 3.44E−07 | NT |
| 20 | 1.12E−07 | NT |
| 21 | 1.03E−07 | NT |
| 22 | 3.0E−09 | 4.0E−09 |
| 23 | 7.46E−09 | 2.77E−09 |
| 24 | 4.0E−09 | 9.9E−09 |
| 25 | 2.9E−09 | 2.3E−09 |
| 26 | 6.1E−09 | 2.0E−08 |
| 27 | 8.8E−09 | 1.7E−08 |
| 28 | 6.41E−08 | 1.38E−07 |
| 29 | 7.1E−09 | 1.8E−09 |
| 30 | 2.84E−08 | 9.1E−09 |
| 31 | 9.15E−09 | 5.45E−09 |
| 32 | 4.6E−09 | NT |
| 33 | 7.77E−09 | 2.55E−09 |
| 34 | 3.58E−09 | 1.1E−08 |
| 35 | 1.13E−08 | 5.0E−09 |
| 36 | 1.51E−08 | 8.93E−09 |
| 37 | 1.36E−08 | 6.66E−09 |
| 38 | 1.4E−08 | 1.1E−08 |
| 39 | 8.2E−09 | 1.3E−08 |
| 40 | 4.19E−09 | 1.7E−09 |
| 41 | 6.7E−09 | 2.5E−09 |
| 42 | 4.1E−09 | 3.66E−09 |
| 43 | 6.42E−09 | 1.2E−09 |
| 44 | 1.81E−08 | 7.4E−09 |
| 45 | 3.6E−09 | 1.4E−09 |
| 46 | 2.9E−09 | 3.1E−09 |
| 47 | 2.2E−09 | 9.2E−10 |
| 48 | 5.2E−09 | 3.0E−09 |
| 49 | 2.49E−08 | 3.1E−08 |
| 50 | 7.89E−09 | 6.17E−09 |
| 51 | 2.6E−09 | 1.4E−09 |
| 52 | 1.3E−08 | 5.4E−09 |
| 53 | 6.6E−09 | 3.4E−09 |
| 54 | 1.3E−08 | 9.1E−09 |
| 55 | 1.5E−08 | 1.2E−08 |
| 56 | 1.6E−08 | 4.2E−08 |
| 57 | 5.1E−09 | NT |
| 58 | 6.51E−09 | NT |
| 59 | 3.58E−08 | 4.11E−08 |
| 60 | 7.59E−08 | 1.28E−07 |
| 61 | 7.9E−09 | 1.7E−08 |
| 62 | 1.4E−08 | 1.4E−08 |
| 63 | 1.24E−08 | 1.63E−08 |
| 64 | 6.4E−09 | 8.39E−09 |
| 65 | 4.2E−09 | 7.66E−09 |
| 66 | 6.5E−09 | 4.5E−09 |
| 67 | 1.4E−08 | 2.78E−08 |
| 68 | 4.0E−09 | 6.67E−09 |
| 69 | 1.81E−08 | 2.17E−08 |
| 70 | 6.3E−08 | 3.4E−08 |
| 71 | 1.12E−07 | NT |
| 72 | 2.35E−07 | NT |
| 73 | 3.26E−07 | NT |
| 74 | 7.8E−08 | 7.0E−08 |
| 75 | 3.9E−08 | 4.3E−08 |
| 76 | 1.7E−08 | 2.2E−08 |
| 77 | 3.7E−08 | 1.3E−08 |
| 78 | 5.8E−08 | 1.5E−08 |
| 79 | 1.21E−07 | NT |
| 80 | 1.9E−08 | 6.2E−09 |
| 81 | 4.4E−08 | 5.1E−09 |
| 82 | 1.6E−08 | NT |
| 83 | 4.2E−08 | 8.5E−09 |
| 84 | 2.1E−08 | 1.8E−08 |
| 85 | 9.9E−09 | 3.4E−08 |
| 86 | 5.8E−08 | 8.38E−07 |
| 87 | 8.8E−08 | 4.5E−08 |
| 88 | 1.87E−07 | NT |
| 89 | 2.0E−08 | 2.6E−08 |
| 90 | 3.45E−07 | NT |
| 91 | 1.3E−08 | 7.6E−08 |
| 92 | 4.9E−09 | 4.57E−08 |
| 93 | 5.1E−08 | 8.64E−08 |
| 94 | 3.0E−08 | 4.9E−08 |
| 95 | 2.1E−08 | 2.7E−08 |
| 96 | 7.21E−09 | 2.73E−08 |
| 97 | 9.8E−09 | 4.27E−08 |
| 98 | 3.05E−09 | 7.0E−09 |
| 99 | 5.6E−08 | 1.2E−07 |
| 100 | 2.4E−08 | 4.76E−08 |
| 101 | 1.1E−08 | 2.53E−08 |
| 102 | 4.1E−09 | 9.31E−09 |
| 103 | 6.4E−09 | 6.63E−09 |
| 104 | 1.5E−08 | 3.72E−08 |
| 105 | 2.6E−09 | 5.08E−09 |
| 106 | 1.5E−08 | 1.86E−08 |
| 107 | 3.3E−09 | 7.64E−09 |
| 108 | 6.3E−08 | 5.0E−08 |
| 109 | 6.5E−08 | 1.1E−08 |
| 110 | 1.82E−07 | NT |
| 111 | 3.12E−07 | NT |
| 112 | 2.03E−07 | NT |
| 113 | 3.06E−08 | NT |
| 114 | 6.6E−08 | 2.2E−08 |
| 115 | 4.18E−07 | NT |
| 116 | 8.0E−09 | NT |
| 117 | 2.86E−08 | 2.76E−08 |
| 118 | 1.23E−07 | NT |
| 119 | 1.53E−09 | 4.3E−09 |
| 120 | 4.54E−08 | 4.13E−08 |
| 121 | 4.68E−09 | 6.4E−09 |
| 122 | 1.36E−08 | 2.3E−08 |
| 123 | 6.92E−09 | 4.32E−08 |
| 124 | 1.2E−08 | 3.15E−08 |
| 125 | 7.98E−09 | 2.66E−08 |
| 126 | 4.44E−09 | 2.95E−08 |
| 127 | 5.93E−09 | 2.04E−08 |
| 128 | 3.86E−08 | 7.5E−08 |
| 129 | 2.87E−09 | 5.2E−09 |
| 130 | 1.83E−09 | 7.5E−09 |
| 131 | 6.4E−11 | 5.3E−09 |
| 132 | 3.54E−08 | 4.78E−08 |
| 133 | 1.52E−09 | 9.8E−09 |
| 134 | 3.7E−09 | 1.7E−08 |
| 135 | 2.06E−07 | NT |
| 137 | 2.26E−07 | NT |
| 138 | 4.5E−08 | 2.4E−08 |
| 139 | 1.9E−08 | 2.46E−08 |
| 140 | 1.2E−08 | 2.0E−08 |
| 143 | 4.2E−08 | 2.17E−07 |
| 144 | 2.2E−08 | 8.02E−08 |
| 147 | 1.9E−08 | 4.87E−08 |
| 148 | 7.5E−09 | 3.99E−08 |
| 149 | 1.4E−08 | 2.41E−08 |
| 150 | 2.19E−08 | NT |
| 151 | 2.14E−07 | NT |
| 152 | 8.2E−08 | 2.02E−07 |
| 153 | 2.87E−08 | 6.2E−08 |
| 154 | 7.8E−09 | 1.14E−08 |
| 155 | 9.7E−08 | 5.88E−07 |
| 156 | 1.4E−07 | NT |
| 157 | 4.83E−09 | 5.18E−09 |
| 158 | 1.07E−08 | 5.24E−09 |
| 159 | 2.22E−08 | 8.18E−09 |
| 160 | 1.69E−08 | 6.47E−09 |
| 161 | 1.01E−08 | 3.63E−09 |
| 162 | 1.27E−08 | 3.72E−09 |
| 163 | 9.76E−09 | 1.41E−09 |
| 164 | 6.48E−09/5.16E−09 | 1.83E−09 |
| 165 | 9.33E−09 | 2.74E−09 |
| 168 | 3.85E−08 | 1.38E−08 |
| 169 | 3.47E−09 | 1.66E−08 |
| 170 | 7.85E−08 | NT |
| 171 | 4.28E−08 | 3.14E−08 |
| 172 | 7.64E−09 | 3.85E−07 |
| 175 | 8.12E−08 | 2.65E−08 |
| 176 | 3.2E−09 | 1.52E−09 |
| 177 | 9.99E−09 | 2.32E−09 |
| 178 | 7.01E−08 | 3.18E−08 |
| 179 | 5.58E−09 | 1.34E−09 |
| 182 | 4.04E−08 | 9.95E−09 |
| 183 | 3.7E−08 | 9.5E−09 |
| 184 | 7.28E−09 | 3.96E−09 |
| 185 | 1.3E−08 | 7.65E−09 |
| 188 | 6.99E−08 | 6.9E−08 |
| 189 | 1.11E−08 | 6.34E−09 |
| 190 | 1.24E−08 | 6.82E−09 |
| 191 | 1.77E−08 | 2.01E−08 |
| 192 | 8.8E−09 | 8.48E−09 |
| 197 | 1.4E−08 | 9.7E−09 |
| 198 | 1.07E−08 | 3.14E−09 |
| 199 | 9.23E−09 | 3.41E−08 |
| 200 | 3.0E−08 | 1.06E−07 |
| 201 | 2.39E−08 | 7.5E−09 |
| 202 | 2.18E−08 | 1.17E−08 |
| 203 | 4.71E−08 | 3.3E−08 |
| 204 | 3.65E−08 | 2.69E−08 |
| 205 | 3.58E−08 | 2.35E−08 |
| 206 | 3.72E−08 | 2.05E−08 |
| 207 | 2.74E−08 | 1.18E−08 |
| 210 | 2.15E−10 | 1.85E−09 |
| 211 | 2.77E−08 | 2.49E−08 |
| 212 | 3.41E−08 | 2.14E−08 |
| 213 | 3.64E−08 | 1.56E−08 |
| 214 | 4.83E−08 | 3.13E−08 |
| 215 | 5.41E−08 | 2.46E−08 |
| 216 | 2.57E−08 | 1.91E−08 |
| 217 | 4.26E−08 | 2.31E−08 |
| 218 | 3.01E−08 | 1.15E−08 |
| 219 | 3.03E−08 | 2.34E−08 |
| 220 | 9.82E−08 | NT |
| 221 | 3.55E−08 | 1.02E−08 |
| 222 | 5.58E−10 | 7.42E−08 |
| 223 | 4.2E−09 | 7.32E−08 |

NT: not tested

### Example C: Pharmaceutical Composition: Tablets

1000 tablets containing a dose of 5 mg of a
compound selected from Examples 1 to 231 5 g

| | | |
|---|---|---|
| Wheat starch | 20 | g |
| Maize starch | 20 | g |
| Lactose | 30 | g |
| Magnesium stearate | 2 | g |
| Silica | 1 | g |
| Hydroxypropylcellulose | 2 | g |

1-31. (canceled)

32. A compound of formula (I): 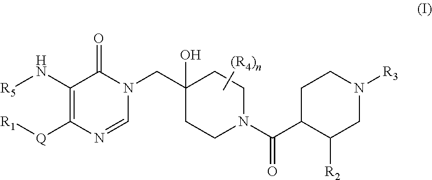
wherein:
Q represents an oxygen atom or a sulphur atom,
R₁ represents a cycloalkyl group, a heterocycloalkyl group, an aryl group, or a heteroaryl group,
R₂ represents an aryl group or a heteroaryl group,
R₃ represents a hydrogen atom, a linear or branched (C₁-C₆)alkyl group, a linear or branched halo(C₁-C₆)alkyl group, a linear or branched hydroxy(C₁-C₆)alkyl group, a —C(O)—R₅ group, a —C(O)—OR₈ group, a —C(O)—NH—R₈ group, or a 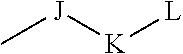
group,
R₄ represents a hydrogen atom or a halogen atom,
R₅ represents a hydrogen atom, a linear or branched (C₁-C₆)alkyl group, a linear or branched halo(C₁-C₆)alkyl group, or an aryl(C₁-C₆)alkyl group,
n is an integer equal to 0, 1 or 2,
J represents a —C(O)— group, a -CH(R₆)— group, at —SO₂— group, a —C(X)—N(R₇)— group, a —CH₂—C(O)—N(R₇)— group, or a 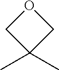
group,
R₆ represents a hydrogen atom, a linear or branched (C₁-C₆)alkyl group, or a —C(O)—OR₈ group,
R₇ represent a hydrogen atom or a linear or branched (C₁-C₆)alkyl group,
R₈ represent a hydrogen atom, a linear or branched (C₁-C₆)alkyl group, or a linear or branched halo(C₁-C₆)alkyl group,
K represents a bond or a -Cy₁- group,
L represents a linear or branched (C₁-C₆)alkyl group, a -Cy₂ group or a —C(R₉)-Cy₂ group,
X represents an oxygen atom or a sulphur atom,
R₉ represents a hydrogen atom or a halogen atom,
Cy₁ represents a cycloalkyl group, a heterocycloalkyl group, an aryl group, or a heteroaryl group, which is linked to the group J and to the group L,
Cy₂ represents a cycloalkyl group, a heterocycloalkyl group, an aryl group, or a heteroaryl group,
wherein:
“aryl” means a phenyl, naphthyl, or indanyl group,
“heteroaryl” means any mono- or fused bi-cyclic group composed of from 5 to 10 ring members, having at least one aromatic moiety and having from 1 to 3 heteroatoms selected from oxygen, sulphur and nitrogen,
“cycloalkyl” means any mono- or fused bi-cyclic non-aromatic carbocyclic group having from 3 to 7 ring members,
“heterocycloalkyl” means any non-aromatic mono- or fused bi-cyclic group having from 3 to 10 ring members, and having from 1 to 3 heteroatoms selected from oxygen, sulphur and nitrogen,
wherein the aryl, heteroaryl, cycloalkyl and heterocycloalkyl groups so defined may be optionally substituted by from 1 to 4 groups selected from linear or branched (C₁-C₆)alkyl, linear or branched (C₂-C₆)alkenyl, linear or branched (C₂-C₆)alkynyl, linear or branched halo(C₁-C₆)alkyl, —Y₁—OR′, —Y₁—NR′R″, —Y₁—S(O)ₘ—R′, oxo, N-oxide (where appropriate), pentafluorosulfide, nitro, —Y₁—CN, —C(O)—R′, —C(O)—OR′, —O—C(O)—R′, —Y₁—C(O)—NR′R″, —Y₁—NR′—C(O)—R″, —Y₁—NR′—C(O)—OR″, halogen, cyclopropyl and pyridinyl which may be optionally substituted by a linear or branched (C₁-C₆)alkyl group,
wherein:
Y₁ represents a bond, a linear or branched (C₁-C₄)alkylene group, or a linear or branched halo(C₁-C₄)alkylene group,
R′ and R″, independently of one another, represent a hydrogen atom, a linear or branched (C₁-C₆)alkyl group, a linear or branched (C₂-C₆)alkenyl group, a linear or branched (C₂-C₆)alkynyl group, a linear or branched (C₁-C₆)alkoxy group, a linear or branched halo(C₁-C₆)alkyl, a linear or branched hydroxy(C₁-C₆)alkyl group, a linear or branched (C₁-C₆)alkoxy(C₁-C₆)alkyl group, a formyl group, a phenyl group, a benzyl group, a cyclopropyl group, a cyclopropylmethyl group, a tetrahydropyranyl group, or the pair (R′, R″), together with the nitrogen atom to which they are attached, forms a non-aromatic ring composed of from 4 to 7 ring members, which may have, in addition to the nitrogen, a second heteroatom selected from oxygen and nitrogen, wherein the second nitrogen may be substituted by from 1 to 2 groups selected from a hydrogen atom and a linear or branched (C₁-C₆)alkyl group,
m is an integer equal to 0, 1 and 2,
its enantiomers, diastereoisomers and addition salts thereof with a pharmaceutically acceptable acid or base.

33. The compound according to claim 32, wherein Q represents an oxygen atom.

34. The compound according to claim 32, wherein R₁ represents an aryl group or a heteroaryl group.

35. The compound according to claim 34, wherein R₁ represents a phenyl group or a pyridinyl group.

36. The compound according to claim 32, wherein R₂ represents an aryl group.

37. The compound according to claim 36, wherein R₂ represents a phenyl group.

38. The compound according to claim 32, wherein R₃ represents a hydrogen atom, a linear or branched hydroxy(C₁-C₆)alkyl group, a —C(O)—OR₈ group, a —C(O)—NH—R₈ group, or a 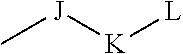
group.

39. The compound according to claim 39, wherein R₃ represents a 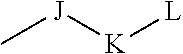
group.

40. The compound according to claim 32, wherein R₄ represents a hydrogen atom or a fluorine atom.

41. The compound according to claim 32, wherein R₅ represents a hydrogen atom.

42. The compound according to claim 32, wherein J represents a —C(O)— group, a —CH₂— group, a —CH|C(O)—O—CH₂-CH₃|— group, a —SO₂— group, a —CH₂—C(O)—N(R₇)— group a —C(X)—N(R₇)— group, or a 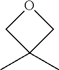
group.

43. The compound according to claim 42, wherein J represents a —C(O)— group, a —CH₂— group, a —SO₂— group, or a —(C(O)—NH— group.

44. The compound according to claim 32, wherein K represents a bond or a -Cy₁- group selected from a phenyl group, a pyrrolyl group, a thienyl group, a thiazolyl group, an oxazolyl group, an imidazolyl group, a pyrazolyl group, a pyridinyl group, a pyrimidinyl group, a dihydrothienodioxinyl group, a cyclopropyl group, or a cyclobutyl group.

45. The compound according to claim 32, wherein L represents a linear or branched (C₁-C₆)alkyl group, a -Cy₂ group, a —CH₂-Cy₂ group, or a -CF₂-Cy₂ group.

46. The compound according to claim 32, wherein Cy₂ represents a cycloalkyl group, a heterocycloalkyl group, an aryl group or a heteroaryl group, which groups are substituted by 1, 2 or 3 groups selected from linear or branched (C₁-C₆)alkyl, linear or branched halo(C₁-C₆)alkyl, —Y₁—OR′, —Y₁—NR′R″, oxo, halogen, wherein R′ and R″, independently of one another, represent a hydrogen atom, or a linear or branched (C₁-C₆)alkyl group, or the pair (R′, R″), together with the nitrogen atom to which they are attached, forms a non-aromatic ring composed of from 5 to 7 ring members, which may have, in addition to the nitrogen, a second heteroatom selected from oxygen and nitrogen, wherein the second nitrogen maybe substituted by a linear or branched (C₁-C₆)alkyl group.

47. The compound according to claim 45, wherein Cy₂ represents a cyclobutyl group, a cyclopentyl group, a cyclohexyl group, a piperidinyl group, a tetrahydropyranyl group, a phenyl group, a pyrrolyl group, a thienyl group, a thiazolyl group, an oxazolyl group, an imidazolyl group, a pyridinyl group, a pyrazinyl group, a pyrimidinyl group, a pyridazinyl group, an indolyl group, a dihydroindolyl group, a isoquinolinyl group, a dihydrothienodioxinyl group, a benzothiazolyl group, or a quinazolinonyl group.

48. The compound according to claim 46, wherein Cy₂ represents a cyclobutyl group, a cyclopentyl group, a cyclohexyl group, a piperidinyl group, a tetrahydropyranyl group, a phenyl group, a pyrrolyl group, a thienyl group, a thiazolyl group, an oxazolyl group, an imidazolyl group, a pyridinyl group, a pyrazinyl group, a pyrimidinyl group, a pyridazinyl group, an indolyl group, a dihydroindolyl group, a isoquinolinyl group, a dihydrothienodioxinyl group, a benzothiazolyl group, or a quinazolinonyl group.

49. The compound according to claim 32, wherein K represents a phenyl group, a pyrrolyl group, a thienyl group, a thiazolyl group, an oxazolyl group, an imidazolyl group, a pyrazolyl group, a pyridinyl group, a pyrimidinyl group, a dihydrothienodioxinyl group, a cyclopropyl group, or a cyclobutyl group and L represents a cyclobutyl group, a cyclopentyl group, a cyclohexyl group, a piperidinyl group, a tetrahydropyranyl group, a phenyl group, a benzyl group, a pyrrolyl group, a thienyl group, or a pyridinyl group.

50. The compound according to claim 32, wherein K represents a bond and L represents a phenyl group, a thienyl group, a thiazolyl group, an oxazolyl group, an imidazolyl group, a pyridinyl group, a pyrazinyl group, a pyrimidinyl group, a pyridazinyl group, an indolyl group, a dihydroindolyl group, a isoquinolinyl group, a dihydrothienodioxinyl group, a benzothiazolyl group, or a quinazolinonyl group.

51. The compound according to claim 32, which is compound of formula (I-a): 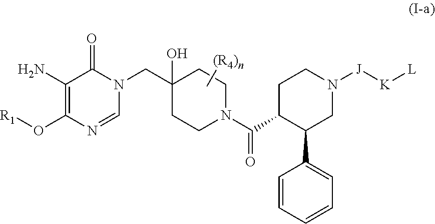
wherein R₁, R₄, J, K, L and n are as defined in claim 32.

52. The compound according to claim 32, which is selected from;
5-amino-3-{[(4S)-3,3-difluoro-4-hydroxy-1- (3R,4?)-1-[(2-methylpyrimidin-4-yl)methyl]-3-phenylpiperidine-4-carbonyl}piperidin-4-yl]methyl}-(4-fluorophenoxy)pyrimidin-4(3 t)-one;
5-amino-6-(4-chlorophenoxy)-3-[(4-hydroxy-1-{(3R,4R)-1-[4-methyl-2-(6-methylpyridin-3-yl)-1,3-thiazole-5-carbonyl-3-phenylpiperidine-4-carbonyl}piperidin-4-yl)methyl]pyrimidin-4(31)-one;
5-amino-6-(3-chloro-5-methoxyphenoxy)-3-[(4-hydroxy-1-(3R,4R)-[[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one;
5-amino-6-(4-chloro-3-fluorophenoxy)-3-[(4-hydroxy-1-(3/R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(3H)-one;
5-amino-6-(4-fluoro-3-methoxyphenoxy)-3-[(4-hydroxy-1-{(3R,4R)-1-[4-methyl-2-(6-methylpyridin-3-yl)-1,3-thiazole-5-carbonyl-3-phenylpiperidine-4-carbonyl}piperidin-4-yl)methyl]pyrimidin-4(3H)-one;
5-amino-3-[(4-hydroxy-1-{(3R,4R)-1-[4-methyl-2-(6-methylpyridin-3-yl)-1,3-thiazole-5-carbonyl-3-phenyl]piperidine-4-carbonyl}piperidin-4-yl}methyl-6-[3-(trifluoromethyl)phenoxy]pyrimidin-4(3H)-one;
5-amino-3-((4-hydroxy-1- (3R,4R)-1-[4-methyl-2-(6-methylpyridin-3-yl)-1,3-thiazole-5-carbonyl-3-phenylpiperidine-4-carbonyl}piperidin-4-yl)methyl]-6-[3-(trifluoromethoxy)phenoxy]pyrimidin-4(3H)-one;
5-amino-3-({1-(3R,4R)-1-(2-bromo-4-methyl-1,3-thiazole-5-carbonyl)-3-phenylpiperidine-4-carbonyl]-4-hydroxypiperidin-4-yl]methyl)-6-(4-fluorophenoxy)pyrimidin-4(3H)-one;
5-amino-3-({1-(3R,4R)-1-(benzenesulfonyl)-3-phenylpiperidine-4-carbonyl-4-hydroxypiperidin-4-yl]methyl)-O-(4-fluorophenoxy)pyrimidin-4(3H)-one;
5-amino-3-({1-(3R,4R)-1-(3-bromobenzoyl)-3-phenylpiperidine-4-carbonyl]-4-hydroxypiperidin-4-yl]methyl)-6-(4-fluorophenoxy)pyrimidin-4(3H)-one;
5-amino-3-({1-[(3R,41R)-1-(5-bromopyridine-3-carbonyl)-3-phenylpiperidine-4-carbonyl]-4-hydroxypiperidin-4-yl}methyl)-6-(4-fluorophenoxy)pyrimidin-4(3H)-one;
5-amino-6-(4-fluorophenoxy)-3-({4-hydroxy-1-[(3R,4R)-1-(6′-methyl]3,3′-bipyridine]-5-carbonyl)-3-phenylpiperidine-4-carbonyl]piperidin-4-yl}methyl)pyrimidin-4(3H)-one;
5-amino-6-(4-fluorophenoxy)-3-(14-hydroxy-1-[(3R,4R)-3-phenyl-1-(5-phenylpyridine-3-carbonyl)piperidine-4-carbonyl]piperidin-4-yl]methyl)pyrimidin-4(3H)-one;
5-amino-3-({1-[(3R,4R)-1-benzoyl-3-phenylpiperidine-4-carbonyl]-4-hydroxypiperidin-4-yl}methyl)-6-(4-fluorophenoxy)pyrimidin-4(3H)-one;
5-amino-6-(4-fluorophenoxy)-3-({4-hydroxy-1-[(3R,4R)-1-(I-methyl-1H-indole-2-carbonyl)-3-phenylpiperidine-4-carbonyl]piperidin-4-yl)methyl)pyrimidin-4(3H)-one;
5-amino-3-({1-[(3R,4R)-1-(3-fluoro-5-iodothiophene-2-carbonyl)-3-phenylpiperidine-4-carbonyl]-4-hydroxypiperidin-4-yl; methyl)-6-(4-fluorophenoxy)pyrimidin-4(31)-one;
5-amino-3-({1-[(3R,4R)-1-[3-fluoro-5-(6-methylpyridin-3-yl)thiophene-2-carbonyl]-3-phenylpiperidine-4-carbonyl]-4-hydroxypiperidin-4-yl)methyl]-6-(4-fluorophenoxy)pyrimidin-4(3H)-one;
5-amino-3-({(4S)-1-[(3R,4R)-1-(2-bromo-4-methyl-1,3-thiazole-5-carbonyl)-3-phenylpiperidine-4-carbonyl]-3,3-difluoro-4-hydroxypiperidin-4-yl}methyl)-6-(4-fluorophenoxy)pyrimidin-4(3H)-one;
5-amino-6-(4-fluorophenoxy)-3-(14-hydroxy-1-[(3R,4R)-1- 4-methyl-2-[6-(trifluoromethyl)pyridin-3-yl]-1,3-thiazole-5-carbonyl]-3-phenylpiperidine-4-carbonyl]piperidin-4-yl methyl)pyrimidin-4(3H)-one;
5-amino-3-({1-](3R,41R)-1-{2-[6-(dimethylamino)pyridin-3-yl-4-methyl-1,3-thiazole-5-carbonyl}-3-phenylpiperidine-4-carbonyl]-4-hydroxypiperidin-4-yl}methyl)-6-(4-fluorophenoxy)pyrimidin-4(3H)-one;
5-amino-3-{[(4S)-3,3-difluoro-1-[(3R,4R)-1-[2-(4-fluorophenyl)-4-methyl-1,3-thiazole-5-carbonyl]-3-phenylpiperidine-4-carbonyl-4-hydroxypiperidin-4-yl]methyl-6-(4-fluorophenoxy)pyrimidin-4(3H)-one;
5-amino-3-[[(4S)-3,3-difluoro-4-hydroxy-1- (3R,4R)-1-[4-methyl-2-(6-methylpyridin-3-yl)-1,3-thiazole-5-carbonyl]-3-phenylpiperidine-4-carbonyl}piperidin-4-yl]methyl}-6-(4-fluorophenoxy)pyrimidin-4(3H)-one;
5-amino-3-[(4-hydroxy-1-((3R,4R)-1-[4-methyl-2-(6-methylpyridin-3-yl)-1,3-thiazole-5-carbonyl-3-phenylpiperidine-4-carbonyl}piperidin-4-yl)methyl-6-phenoxypyrimidin-4(3H)-one;
5-amino-6-(4-fluorophenoxy)-3-[(4-hydroxy-1- (3R,4R)-1-[4-methyl-2-(6-methylpyridin-3-yl)-1,3-thiazole-5-carbonyl]-3-phenylpiperidine-4-carbonyl}piperidin-4-yl)methyl]pyrimidin-4(31)-one;
5-amino-6(4-fluorophenoxy)-3-1 (1-((3R,4R)-1,2-(4-fluorophenyl)-4-methyl-1,3-thiazole-5-carbonyl]-3-phenylpiperidine-4-carbonyl)-4-hydroxypiperidin-4-yl)methyl]pyrimidin-4(3)-one;
5-amino-3-[[1-[(3R,4R)-1-[(2-bromothiazol-5-yl)methyl-3-phenyl-piperidine-4-carbonyl-4-hydroxy-4-piperidyl]methyl-6-(4-fluorophenoxy)pyrimidin-4-one;
5-amino-6-(4-fluorophenoxy)-3-(14-hydroxy-1-[(3R,4R)-I-(2-(6-methylpyridin-3-yl)-1,3-thiazol-5-yl]methyl)-3-phenylpiperidine-4-carbonyl]piperidin-4-yl)methyl)pyrimidin-4(3H)-one;
5-amino-6-(4-fluorophenoxy)-3-[(1-((3R,4R)-1-[(2-fluorophenyl)methyl]-3-phenylpiperidine-4-carbonyl-4-hydroxypiperidin-4-yl)methyl]pyrimidin-4(3H)-one;
5-amino-3-({1(4S′)-3,3-difluoro-4-hydroxy-1-[(3R,4R)-1-[2-(6-methylpyridin-3-yl)-1,3-thiazol-5-yl}methyl-3-phenylpiperidine-4-carbonyl]piperidin-4-yl]methyl)-6-(4-fluorophenoxy)pyrimidin-4(3H)-one;
5-amino-3-{[(4S)-3,3-difluoro-[(3R,4R)-1-[(2-fluorophenyl)methyl]-3-phenylpiperidine-4-carbonyl-4-hydroxypiperidin-4-yl]methyl}-6(4-fluorophenoxy)pyrimidin-4(3H)-one;
5-amino-3-({1-[(3R,4R)-1-(3-ethoxybenzoyl)-3-phenylpiperidine-4-carbonyl}-4-hydroxypiperidin-4-yl]methyl)-6-(4-fluorophenoxy)pyrimidin-4(3H)-one;
5-amino-3-({1-[(3R,4R)-1-(2-benzyl-4-methyl-1,3-thiazole-5-carbonyl)-3-phenylpiperidine-4-carbonyl-4-hydroxypiperidin-4-yl}methyl)-6-(4-fluorophenoxy)pyrimidin-4(3H)-one;
5-amino-6-(4-fluorophenoxy)-3-[(4-hydroxy-1-((3R,4R)-3-phenyl-1-1-(pyridin-3-yl)methyl]piperidine-4-carbonyl}piperidin-4-yl)methyl]pyrimidin-4(3H-one;
5-amino-6-(4-fluorophenoxy)-3-[(4-hydroxy-1-{(3R,4R)-1-[4-methyl-2-(morpholin-4-yl)-1,3-thiazole-5-carbonyl]-3-phenylpiperidine-4-carbonyl}piperidin-4-yl)methyl]pyrimidin-4(310-one;
5-amino-6-(4-fluorophenoxy)-3-[(4-hydroxy-1-((3R,4R)-1-[4-methyl-2-(pyrrolidin-1-yl)-1,3-thiazole-5-carbonyl]-3-phenylpiperidine-4-carbonyl}piperidin-4-yl))methyl]pyrimidin-4(3H)-one;
5-amino-6-(4-fluorophenoxy)-3-({4-hydroxy-1-[(13R,41?)-1-(4-methyl-2-phenyl-1,3-thiazole-5-carbonyl)-3-phenylpiperidine-4-carbonyl]piperidin-4-yl}methyl)pyrimidin-4(3H)-one;
5-amino-6-(4-fluorophenoxy)-3-[(4-hydroxy-1-{(3R,41R)-1-[7-(6-methylpyridin-3-yl)-2,3-dihydrothieno[3,4-b][1,4]dioxine-5-carbonyl]-3-phenylpiperidine-4-carbonyl}piperidin-4-yl)methyl]pyrimidin-4(3H)-one;
(3R,4R)-4-(4-{[5-amino-4-(4-fluorophenoxy)-6-oxopyrimidin-[(6H)-yl]methyl-4-hydroxypiperidine-1-carbonyl)-N-(4-methoxyphenyl)-3-phenylpiperidine-1-carboxamide;
(3R,4R)-4-(4-([5-amino-4-(4-fluorophenoxy)-6-oxopyrimidin-[(6H)-yl]methyl)-14-hydroxypiperidine-1-carbonyl)-3-phenyl-N-[3-(trifluoromethyl)phenyl]piperidine-1-carboxamide;
(3R,4R)-4-(4-(5-amino-4-(4-fluorophenoxy)-6-oxopyrimidin-[(6H)-yl]methyl}4-hydroxypiperidine-1-carbonyl)-V-(3-bromophenyl)-3-phenylpiperidine-1-carboxamide;
(3R,4R)-4-[4-[[5-amino-4-(4-fluorophenoxy)-6-oxo-pyrimidin-1-yl]methyl-4-hydroxy-piperidine-1-carbonyl]-N-[3-(6-methyl-3-pyridyl)phenyl]-3-phenyl-piperidine-1-carboxamide;
5-amino-6-(4-fluorophenoxy)-3-({4-hydroxy-1-[(3R,4R)-1-(5-methylpyridine-3-carbonyl)-3-phenylpiperidine-4-carbonyl]piperidin-4-yl}methyl)pyrimidin-4(3H)-one;
5-amino-6-(4-fluorophenoxy)-3-[(4-hydroxy-1-[(3R,4R)-1-[1-methyl-2-(6-methylpyridin-3-yl)-1H-imidazole-5-carbonyl]-3-phenylpiperidine-4-carbonyl]piperidin-4-yl)methyl]pyrimidin-4(3H)-one;
5-amino-3-({I-{(3R,4R)-1-(2,6-dimethylpyridine-4-carbonyl)-3-phenylpiperidine-4-carbonyl]-4-hydroxypiperidin-4-yl}methyl)-6-(4-fluorophenoxy)pyrimidin-4(3H)-one;
5-amino-3-({1-[(3R,4R)-1-(3-bromo-5-(fluorobenzoyl)-3-phenylpiperidine-4-carbonyl]-4-hydroxypiperidin-4-yl}methyl)-6-(4-fluorophenoxy)pyrimidin-4(3H)-one;
5-amino-6-(4-fluorophenoxy)-3-(14-hydroxy-1-[(3R,4R)-1-(isoquinoline-5-carbonyl)-3-phenylpiperidine-4-carbonyl]piperidin-4-yl}methyl)pyrimidin-4(3H)-one;
5-amino-3-({(4S)-3,3-difluoro-4-hydroxy-1-[(3R,4R)-1-[4-methyl-2-[6-(trifluoromethyl)pyridin-3-yl]-3-thiazole-5-carbonyl]-3-phenylpiperidine-4-carbonyl]piperidin-4-yl]methyl)-6-(4-fluorophenoxy)pyrimidin-4(3H)-one;
5-amino-3-[[(4S)-3,3-difluoro-1-{(3R,4R)-1-[3-fluoro-5-(6-methylpyridin-3-yl)thiophene-2-carbonyl-3-phenylpiperidine-4-carbonyl)-4-hydroxypiperidin-4-yl]methyl}-6-(4-fluorophenoxy)pyrimidin-4(3H)-one;
5-amino-3-[(1-{(3R,4R)-1-[5-chloro-3-fluoro-4-(6-methylpyridin-3-yl)thiophene-2-carbonyl]-3-phenylpiperidine-4-carbonyl-4-hydroxypiperidin-4-yl)methyl]-6-(4-fluorophenoxy)pyrimidin-4(3H)-one;
5-amino-3-([(4S)-3,3-difluoro-4-hydroxy-1- ((3R,4R)-1-[4-methyl-2-(6-methylpyridin-3-yl)-1,3-thiazole-5-carbonyl]-3-phenylpiperidine-4-carbonyl) piperidin-4-yl]methyl-6-phenoxypyrimidin-4(3H)-one;
5-amino-3-[(1-[(3R,4R)-1-[3-fluoro-5-(6-methylpyridin-3-yl)benzoyl]-3-phenylpiperidine-4-carbonyl-4-hydroxypiperidin-4-yl)methyl]-6-(4-fluorophenoxy)pyrimidin-4(3H)-one;
6-[(3R,4R)-4-(4-{[5-amino-4-(4-fluorophenoxy)-6-oxopyrimidin-[(6H)-yl]methyl]-4-hydroxypiperidine-1-carbonyl)-3-phenylpiperidine-1-carbonyl]-3-methylquinazolin-4(3H)-one;
5-amino-3-[[4-hydroxy-1-[3R,4R)-1-[4-methyl-2-[6-(trifluoromethyl)-3-piperidyl]thiazole-5-carbonyl]-3-phenyl-piperadine-4-carbonyl]-4-piperidyl]methyl]-6-[3-(trifluoromethyl)phenoxy]pyrimidin-4-one;
5-amino-3-[[1-[(3R,4R)-1-[2-[6-(dimethylamino)-3-pyridyl]-4-methyl-thiazole-5-carbonyl]-3-phenyl-piperidine-4-carbonyl]-4-hydroxy-4-piperidyl]methyl-6-[3-(trifluoromethyl)phenoxy]pyrimidin-4-one;
5-amino-3-[[1-[(3R,4R)-1-(3-chloro-1-methyl-indole-2-carbonyl)-3-phenyl-piperidine-4-carbonyl]-4-hydroxy-4-piperidyl]methyl]6-(4-fluorophenoxy)pyrimidin-4-one;
5-amino-3-[[1-[(3R,4R)-1-(3,5-difluorobenzoyl)-3-phenyl-piperidine-4-carbonyl]-4-hydroxy-4-piperidyl]methyl]-6-(4-fluorophenoxy)pyrimidin-4-one;
5-amino-3-[[1-[(3R,4R)-1-(3,5-dichlorobenzoyl)-3-phenyl-piperidine-4-carbonyl]-4-hydroxy-4-piperidyl]methyl]-6-(4-fluorophenoxy)pyrimidin-4-one;
5-amino-6-[4-(aminomethyl)phenoxy-3-[[4-hydroxy-1-[(3R,4R)-1-[4-methyl-2-(6-ethyl-3-pyridyl)thiazole-5-carbonyl]-3-phenyl-piperidine-4-carbonyl]-4-piperidyl]methyl]pyrimidin-4-one;
5-amino-3-[[1-(3R,4R)-1-(5-chloropyridine-3-carbonyl)-3-phenyl-piperidine-4-carbonyl]-4-hydroxy-4-piperidyl]methyl]-6-(4-fluorophenoxy)pyrimidin-4-one;
5-amino-6-(4-fluorophenoxy)-3-[[1-[(3R,4R)-1-(5-fluoropyridine-3-carbonyl)-3-phenyl-piperidine-4-carbonyl]-4-hydroxy-4-piperidyl]methyl]pyrimidin-4-one;
5-amino-6-(4-chloro-3-fluoro-phenoxy)-3-[[(4S)-3,3-difluoro-4-hydroxy-1-(3R,4R)-1-[(5-methylpyrazin-2-yl)methyl]-3-phenyl-piperidine-4-carbonyl]-4-piperidyl]methyl]pyrimidin-4-one;
5-amino-3-[[(4S)-3,3-difluoro-4-hydroxy-1-[(3R,4R)-1-[(5-methylpyrazin-2-yl)methyl]-3-phenyl-piperidine-4-carbonyl]-4-piperidyl]methyl-6-(4-fluoro-3-methoxy-phenoxy)pyrimidin-4-one;
5-amino-3-[[(4S)-3,3-difluoro-4-hydroxy-1-[(3R,4R)-1-(5-methylpyridine-3-carbonyl)-3-phenyl-piperidine-4-carbonyl]-4-piperidylmethyl]-6-(4-fluorophenoxy)pyrimidin-4-one;
5-amino-3-[[1-[(3R,4R)-1-(2-cyclohexyl-4-methyl-thiazole-5-carbonyl)-3-phenyl-piperidine-4-carbonyl]-4-hydroxy-4-piperidyl]methyl-6-(4-fluorophenoxy)pyrimidin-4-one;
5-amino-6-(4-fluorophenoxy)-3-[[4-hydroxy-1-[(3R,4R)-1-[3-(6-methyl-3-pyridyl)phenyl]sulfonyl-3-phenyl-piperidine-4-carbonyl]4-piperidyl]methyl]pyrimidin-4-one;
5-amino-3-[[(4S)-1-[(3R,4R)-1-[(2-chloropyrimidin-4-yl)methyl]-3-phenyl-piperidine-4-carbonyl]-3,3-difluoro-4-hydroxy-4-piperidyl]methyl]-6-(4-fluorophenoxy)pyrimidin-4-one:
5-amino-3-[[1-1(3R,4R)-1-(2-cyclopentyl-4-methyl-thiazole-5-carbonyl)-3-phenyl-piperidine-4-carbonyl]-4-hydroxy-4-piperidyl]methyl]-6-(4-fluorophenoxy)pyrimidin-4-one;
5-amino-3-[[1-1(3R,4R)-1-(3-chloro-5-methyl-benzoyl)-3-phenyl-piperidine-4-carbonyl]-4-hydroxy-4-piperidyl]methyl]-6-(4-fluorophenoxy)pyrimidin-4-one;
5-amino-6-(4-fluorophenoxy)-3-[[4-hydroxy-1-1 (3R,4R)-3-phenyl-1-(5-pyrrazole-1-ylpyridine-3-carbonyl)piperidine-4-carbonyl]-4-piperidyl]methyl]pyrimidin-4-one;
5-amino-3-[1(4S)-3,3-di fluoro-4-hydroxy-1-1(3R,4R)-1-[16-(6-methyl-3-pyridyl)pyrimidin-4-yl]methyl]-3-phenyl-piperidine-4-carbonyl]-4-piperidyl]methyl]-6-(4-fluorophenoxy)pyrimidin-4-one:
5-amino-3-[[1-1(3R,4R)-1-(2-cyclobutyl-4-methyl-thiazole-5-carbonyl)-3-phenyl-piperidine-4-carbonyl]-4-hydroxy-4-piperidyl]methyl]-6-(4-fluorophenoxy)pyrimidin-4-one;
5-amino-6-(4-chlorophenoxy)-3-1[(4S)-3,3-difluoro-4-hydroxy-1-1(3R,4R)-1-[(2-methylpyrimidin-4-yl)methyl]-3-phenyl-piperidine-4-carbonyl]-4-piperidyl]methyl]pyrimidin-4-one:
5-amino-3-[[4-hydroxy-1-[(3R,4R)-1-[4-methyl-2-((6-methyl-3-pyridyl)thiazole-5-carbonyl]-3-phenyl-piperidine-4-carbonyl]-4-piperidyl]methyl]-6-(3-pyridyloxy)pyrimidin-4-one;
5-amino-3-[[(4S)-3,3-difluoro-4-hydroxy-1-1(3R,4R)-1-[[2-(6-methyl-3-pyridyl)pyrindine-4-yl]methyl]-3-phenyl-piperidine-4-carbonyl]-4-piperidyl]methyl]-6-(4-fluorophenoxy)pyrimidin-4-one:
5-amino-3-[[(4S)-3,3-difluoro-4-hydroxy-1-1(3R,4R)-3-phenyl-1-1(2-phenyl]pyrimidin-4-yl)methyl]piperidine-4-carbonyl]-4-piperidyl]methyl]-6-(4-fluorophenoxy)pyrimidin-4-one:
amino-3-[[(4S)-3,3-difluoro-4-hydroxy-1-1(3R,4R)-1-[4-methyl-2-(6-methyl-3-pyridyl)thiazole-5-carbonyl]-3-phenyl-piperidine-4-carbonyl]-4-piperidyl]methyl]-6-(4-fluoro-3-methoxy-phenoxy)pyrimidin-4-one:
5-amino-6-(4-chloro-3-methoxy-phenoxy)-3-[[(4S)-3,3-di fluoro-4-hydroxy-1-[(3R,4R)-1-[(2-methylpyrimidin-4-yl)methyl]-3-phenyl-piperidine-4-carbonyl]-4-piperidyl]methyl]pyrimidin-4-one:
5-amino-6-(4-chloro-3-methoxy-phenoxy)-3-[[(4S)-3,3-difluoro-4-hydroxy-1-[(3R,4R)-1-1(5-methylpyrazin-2-yl)methyl 1-3-phenyl-piperidine-4-carbonyl]-4-piperidyl]methyl]pyrimidin-4-one:
5-amino-6-(4-chloro-3-methoxy-phenoxy)-3-1H4S)-3,3-di fluoro-4-hydroxy-1-[(3R,4R)-1-[4-methyl-2-(6-methyl-3-pyridyl)thiazole-5-carbonyl]-3-phenyl-piperidine-4-carbonyl]-4-piperidyl]methyl]pyrimidin-4-one,
5-amino-3-[[(4S)-3,3-di fluoro-4-hydroxy-1-[(3R,4R)-1-[4-methyl-2-[6-methyl-5-(trifluoromethyl)-3-pyridyl]thiazole-5-carbonyl]-3-phenyl-piperidine-4-carbonyl]-4-piperidyl]methyl]-6-(4-fluorophenoxy)pyrimidin-4-one;
5-amino-3-[[1-[(3R,4R)-1-[2-(3,3-difluorocyclobutyl)-4-methyl-thiazole-5-carbonyl]-3-phenyl-piperidine-4-carbonyl]-4-hydroxy-4-piperidyl]methyl]-6-(4-fluorophenoxy)pyrimidin-4-one:
5-amino-3-[[(4S)-1-[(3R,4R)-fluorocyclobutyl)-4-methyl-thiazole-5-carbonyl]-3-phenyl-piperidine-4-carbonyl]-3,3-difluoro-4-hydroxy-4-piperidyl]methyl]-6-(4-fluorophenoxy)pyrimidin-4-one;
5-amino-3-[[(4S)-3,3-difluoro-4-hydroxy-1-[(3R,4R)-1-(4-methyl-2-tetrahydropyran-4-yl-thiazole-5-carbonyl)-3-phenyl-piperidine-4-carbonyl]-4-piperidyl]methyl]-6-(4-fluorophenoxy)pyrimidin-4-one:
5-amino-3-[[(4S)-3,3-difluoro-4-hydroxy-I-[(3R,4R)-1-1(5-methylpyrazin-2-yl)methyl]-3-phenyl-piperidine-4-carbonyl]-4-piperidyl]methyl]-6-(4-fluorophenoxy)pyrimidin-4-one:
5-amino-3-[[(4S)-3,3-difluoro-4-hydroxy-1-[(3R,4R)-3-phenyl-1-(pyrazin-2-ylmethyl)piperidine-4-carbonyl]-4-piperidyl]methyl]-6-(4-fluorophenoxy)pyrimidin-4-one;
5-amino-3-[[(4S)-3,3-difluoro-4-hydroxy-1-1(3R,4R)-1-[(6-methylpyrazin-2-yl)methyl]-3-phenyl-piperidine-4-carbonyl]-4-piperidyl]methyl]-6-(4-fluorophenoxy)pyrimidin-4-one:
5-amino-6-[4-(aminomethyl)phenoxy]-3-[[(4S)-3,3-difluoro-4-hydroxy-1-1(3R,4R)-1-[4-methyl-2-(6-methyl-3-pyridyl)thiazole-5-carbonyl]-3-phenyl-piperidine-4-carbonyl]-4-piperidyl]methyl]pyrimidin-4-one;
5-amino-3-[[(4S)-3,3-difluoro-4-hydroxy-1-1(3R,4R)-1-[(3-methylpyrazin-2-yl)methyl]-3-phenyl-piperidine-4-carbonyl]-4-piperidyl)methyl]-6-(4-fluorophenoxy)pyrimidin-4-one;
5-amino-3-[[(4S)-3,3-difluoro-4-hydroxy-1-[(3R,4R)-3-phenyl-pyrimidin-4-ylmethyl)piperidine-4-carbonyl]-4-piperidyl]methyl]-6-(4-fluorophenoxy)pyrimidin-4-one;
5-amino-6-(4-chlorophenoxy)-3-[[(4S)-3,3-difluoro-4-hydroxy-1-1(3R,4R)-1-1(5-methylpyrazin-2-yl)methyl]-3-phenyl-piperidine-4-carbonyl]-4-piperidyl]methyl]pyrimidin-4-one; and
5-amino-6-[4-(aminomethyl)phenoxyl-3-[[(4S)-1-1(3R,4R)-1-(3,5-dichlorobenzoyl)-3-phenyl-piperidine-4-carbonyl]-3,3-difluoro-4-hydroxy-4-piperidyl]methyl]pyrimidin-4-one.

53. A pharmaceutical composition comprising the compound according to claim 32, or an addition salt thereof with a pharmaceutically acceptable acid or base, in combination with one or more pharmaceutically acceptable excipients.

54. A method of treating a condition requiring a pro-apoptotic and/or anti-proliferative agent in a subject in need thereof, comprising administration of the compound according to claim 32, alone or in combination with one or more pharmaceutically acceptable excipients.

55. The method according to claim 54, wherein the condition is selected from cancer, auto-immune diseases, and immune system diseases.

56. The method according to claim 55, wherein the condition is selected from cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas, ovarian cancer, non-small-cell lung cancer, prostate cancer, pancreatic cancer and small-cell lung cancer.

57. A combination comprising the compound according to claim 32 and an anti-cancer agent selected front genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies.

58. A pharmaceutical composition comprising the combination according to claim 57 in combination with one or more pharmaceutically acceptable excipients.

59. A method of treating cancer in a subject in need thereof, comprising administration of the combination according to claim 57, alone or in combination with one or more pharmaceutically acceptable excipients.

60. A method of treating cancer requiring radiotherapy in a subject in need thereof, comprising administration of the compound according to claim 32, alone or in combination with one or more pharmaceutically acceptable excipients.
